SEQUENCE LISTING (36 sequences)

SEQ ID NO: 1 - DNA, 20 bp, Artificial Sequence
  Synthetic primer
gctctctgcccgtctatacc

SEQ ID NO: 2 - DNA, 24 bp, Artificial Sequence
  Synthetic primer
gggctggtttcagaatagtcaact

SEQ ID NO: 3 - DNA, 21 bp, Artificial Sequence
  Synthetic primer
tgaggtcacggacgattacat

SEQ ID NO: 4 - DNA, 22 bp, Artificial Sequence
  Synthetic primer
gtaggcccacgaaacaaatgat

SEQ ID NO: 5 - DNA, 23 bp, Artificial Sequence
  Synthetic primer
tttgtcaacttgagtcccttcac

SEQ ID NO: 6 - DNA, 21 bp, Artificial Sequence
  Synthetic primer
tcccgctacacttgttttcac

SEQ ID NO: 7 - DNA, 19 bp, Artificial Sequence
  Synthetic primer
aaggcggtgacctcacaag

SEQ ID NO: 8 - DNA, 22 bp, Artificial Sequence
  Synthetic primer
aaagtccaattcctcgatggtg

SEQ ID NO: 9 - DNA, 21 bp, Artificial Sequence
  Synthetic primer
aaactcgcatctactggcaaa

SEQ ID NO: 10 - DNA, 21 bp, Artificial Sequence
  Synthetic primer
ggttcttgtactcgggccata

SEQ ID NO: 11 - DNA, 21 bp, Artificial Sequence
  Synthetic primer
agaagagcgaccctcacatca

SEQ ID NO: 12 - DNA, 21 bp, Artificial Sequence
  Synthetic primer
cggttagcacacactcctttg

SEQ ID NO: 13 - DNA, 21 bp, Artificial Sequence
  Synthetic primer
aaggtgaaggtcggagtcaac

SEQ ID NO: 14 - DNA, 22 bp, Artificial Sequence
  Synthetic primer
ggggtcattgatggcaacaata

SEQ ID NO: 15 - DNA, 21 bp, Artificial Sequence
  Synthetic primer
atcgtggggcttctcatgttt

SEQ ID NO: 16 - DNA, 23 bp, Artificial Sequence
  Synthetic primer
ttagtcatgcccaacttcacttt

SEQ ID NO: 17 - DNA, 23 bp, Artificial Sequence
  Synthetic primer
agtccctgtgctaggatttttca

SEQ ID NO: 18 - DNA, 22 bp, Artificial Sequence
  Synthetic primer
acataaactcgcctgattggtc

SEQ ID NO: 19 - DNA, 23 bp, Artificial Sequence
  Synthetic primer
atgatggcttattacagtggcaa

SEQ ID NO: 20 - DNA, 20 bp, Artificial Sequence
  Synthetic primer
gtcggagattcgtagctgga

SEQ ID NO: 21 - DNA, 23 bp, Artificial Sequence
  Synthetic primer
actgagagtgattgagagtggac

SEQ ID NO: 22 - DNA, 20 bp, Artificial Sequence
  Synthetic primer
aacctctgcacccagttttc

SEQ ID NO: 23 - DNA, 21 bp, Artificial Sequence
  Synthetic primer
gcgtggtgttgctaaccttca

SEQ ID NO: 24 - DNA, 20 bp, Artificial Sequence
  Synthetic primer
aaggccacggtcatcagagt

SEQ ID NO: 25 - DNA, 20 bp, Artificial Sequence
  Synthetic primer
gtactcgacctgtaccagcg

SEQ ID NO: 26 - DNA, 20 bp, Artificial Sequence
  Synthetic primer
tcagggcgaggaccatagag

SEQ ID NO: 27 - DNA, 21 bp, Artificial Sequence
  Synthetic primer
ctcgatccgctcctttgatga

SEQ ID NO: 28 - DNA, 19 bp, Artificial Sequence
  Synthetic primer
cgttggtgcggtctatgag

SEQ ID NO: 29 - DNA, 25 bp, Artificial Sequence
  Synthetic primer
gcccacatcaaggagtatttctaca

SEQ ID NO: 30 - DNA, 19 bp, Artificial Sequence
  Synthetic primer
cggttctttcgggtgacaa

SEQ ID NO: 31 - DNA, 20 bp, Artificial Sequence
  Synthetic primer
accgaggcttcaccaagatg

SEQ ID NO: 32 - DNA, 21 bp, Artificial Sequence
  Synthetic primer
catcatagacgcgacctgtca

SEQ ID NO: 33 - DNA, 22 bp, Artificial Sequence
  Synthetic primer
cctctctctaatcagccctctg

SEQ ID NO: 34 - DNA, 21 bp, Artificial Sequence
  Synthetic primer
gaggacctgggagtagatgag

SEQ ID NO: 35 - DNA, 21 bp, Artificial Sequence
  Sythetic primer
agggcagaatcatcacgaagt

SEQ ID NO: 36 - DNA, 20 bp, Artificial Sequence
  Sythetic primer
agggtctcgattggatggca

(19) United States
(12) Patent Application Publication
(10) Pub. No.: US 2016/0199450 A1
(43) Pub. Date: Jul. 14, 2016

(54) SCAFFOLDS CONTAINING CYTOKINES FOR TISSUE ENGINEERING

(71) Applicant: THE TRUSTEES OF COLUMBIA UNIVERSITY IN THE CITY OF NEW YORK, New York, NY (US)

(72) Inventors: Gordana Vunjak-Novakovic, New York, NY (US); Kara Lorraine Spiller, Philadelphia, PA (US)

(73) Assignee: The Trustees of Columbia University in the City of New York, New York, NY (US)

(21) Appl. No.: 14/914,420

(22) PCT Filed: Aug. 26, 2014
(86) PCT No.: PCT/US2014/052730

§ 371 Date: Feb. 25, 2016

Related U.S. Application Data
(60) Provisional application No. 61/870,213, filed on Aug. 26, 2013.

(51) Int. Cl. A61K 38/21; A61K 38/20; A61K 31/739

(52) U.S. Cl. CPC A61K 38/217; A61K 31/739; A61K 38/2026; A61K 38/2066

(57) ABSTRACT

The present disclosure provides biocompatible scaffold that promotes M1 or M2 macrophage phenotypes so as to increase vascularization or healing. Also provided are methods of treating a subject in need with the scaffolds described here.

### CROSS-REFERENCE TO RELATED APPLICATIONS

The present application claims the benefit of U.S. Provisional Application Ser. No. 61/870,213, filed 26 Aug. 2013, which is incorporated herein by reference in its entirety.

### STATEMENT REGARDING FEDERALLY SPONSORED RESEARCH OR DEVELOPMENT

This invention was made with government support under grant number EB002520 awarded by National Institutes of Health. The government has certain rights in the invention.

### BACKGROUND OF THE INVENTION

Angiogenesis is understood to be crucial for the success of most tissue engineering strategies. The natural inflammatory response is a major regulator of vascularization, through the activity of different types of macrophages and the cytokines they secrete. Macrophages exist on a spectrum of diverse phenotypes, from “classically activated” M1 to “alternatively activated” M2 macrophages. M2 macrophages, including the subsets M2a and M2c, are typically considered to promote angiogenesis and tissue regeneration, while M1 macrophages are considered to be inflammatory.

### SUMMARY OF THE INVENTION

Among the various aspects of the present disclosure is the provision of a biocompatible scaffold that produces increased vascularization compared to a conventional scaffold. In some embodiments, the scaffold includes a matrix material; a first composition that promotes an M1 macrophage phenotype; and a second composition that promotes an M2 macrophage phenotype. In some embodiments, the scaffold promotes an increased level vascularization when in fluid communication with cells in vitro or in vivo compared to a scaffold not comprising the first composition and the second composition.

In some embodiments, the first composition comprises interferon-gamma (IFNy) or Tumor necrosis factor alpha (TNFα) and promotes an M1 macrophage phenotype. In some embodiments, the second composition comprises interleukin-4 (IL4), interleukin-13 (IL13), or interleukin-10 (IL10) and promotes an M2 macrophage phenotype. In some embodiments, the scaffold includes a second composition having interleukin-4 (IL4) or interleukin-13 (IL13), where the second composition promotes an M2A macrophage phenotype. In some embodiments, the scaffold further includes a third composition having Interleukin-10 (IL10), where the third composition promotes an M2C macrophage phenotype.

In some embodiments, the first composition is released prior to the second composition or the third composition (when present). In some embodiments, promotion of the M1 macrophage phenotype is temporally separated from promotion of the M2 macrophage phenotype. In some embodiments, an effect of the M1 macrophage phenotype occurs prior to an effect of the M2 macrophage phenotype.

In some embodiments, the first composition, the second composition, or the third composition (when present) is bound (e.g., releasably bound) to the matrix. In some embodiments, the first composition, the second composition, or the third composition (when present) is adsorbed into or onto the matrix but not covalently bound. In some embodiments, at least one of the first composition is adsorbed into or onto the matrix but not covalently bound; the second composition or the third composition (when present) is releasably bound to the matrix; and the first composition is released prior to the second composition or the third composition (when present).

In some embodiments, IFNy is present in the scaffold at concentration of about 100 ng/ml. In some embodiments, TNFα is present in the scaffold at concentration of about 100 ng/ml. In some embodiments, IL4 is present in the scaffold at concentration of about 40 ng/ml. In some embodiments, IL13 is present in the scaffold at concentration of about 20 ng/ml. In some embodiments, IL10 is present in the scaffold at concentration of about 40 ng/ml.

In some embodiments, the first composition, the second composition, or the third composition (when present) is formulated as a controlled release composition. In some embodiments, the first composition, the second composition, or the third composition (when present) is encapsulated in a polymeric microsphere or a liposome.

In some embodiments, the scaffold includes cells. In some embodiments, the scaffold includes progenitor cells. In some embodiments, the scaffold includes cells selected from the group consisting of mesenchymal stem cells (MSC), MSC-derived cells, osteoblasts, chondrocytes, myocytes, adipocytes, neurons, glial cells, fibroblasts, cardiomyocytes, liver cells, kidney cells, bladder cells, beta-pancreatic islet cell, odontoblasts, dental pulp cells, periodontal cells, tenocytes, lung cells, cardiac cells, hematopoietic stem cells (HSC), HSC endothelial cells, blood vascular endothelial cells, lymph vascular endothelial cells, cultured endothelial cells, primary culture endothelial cells, bone marrow stem cells, cord blood cells, human umbilical vein endothelial cell (HUVEC), lymphatic endothelial cell, endothelial progenitor cell, stem cells that differentiate into an endothelial cells, smooth muscle cells, interstitial fibroblasts, and myofibroblasts, or a combination thereof. In some embodiments, the scaffold includes cells present in the matrix at a density of at least about 0.0001 million cells (M) ml⁻¹ up to about 1000 M ml⁻¹.

In some embodiments, the matrix is wholly or partially composed of a material selected from the group consisting of fibrin, fibrinogen, a collagen, a polyorthoester, a polyvinyl alcohol, a polyamide, a polycarbonate, a polyvinyl pyrrolidone, a marine adhesive protein, a cyanoacrylate, and a polymeric hydrogel, or a combination thereof.

Another aspect provides a method of treating a subject with a scaffold described herein. For example, a subject can be treated for a tissue or organ defect. In some embodiments, the method includes placing a scaffold described herein into fluid communication with cells of a subject in need thereof. In some embodiments, the scaffold produces an increased level vascularization compared to a scaffold not comprising the first composition, the second composition, or the third composition (when present). In some embodiments, the method further includes incubating a cell-containing scaffold in vitro.

In some embodiments of the method, the first composition comprises interferon-gamma (IFNy) or Tumor necrosis factor alpha (TNFα) and promotes an M1 macrophage phenotype; the second composition comprises interleukin-4 (IL4) or interleukin-13 (IL13) and promotes an M2A macrophage phenotype; and the third composition comprises Interleukin-10 (IL10) and promotes an M2C macrophage phenotype. In some embodiments of the method, the first composition is released prior to the second composition or the third composition; promotion of the M1 macrophage phenotype is temporally separated from promotion of the M2A macrophage phenotype or the M2C macrophage phenotype; or an effect of the M1 macrophage phenotype occurs prior to an effect of he M2A macrophage phenotype or the M2C macrophage phenotype.

In some embodiments of the method, the subject is a horse, cow, dog, cat, sheep, pig, mouse, rat, monkey, hamster, guinea pig, and chicken, or human.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a cartoon and a series of bar graphs showing derivation and characteristics of macrophages. In FIG. 1A, peripheral blood monocytes were differentiated to macrophages (M0) and polarized to 3 different phenotypes (M1, M2a, M2c). In FIG. 1B shows M1 macrophages upregulated pro-inflammatory proteins IL1β and TNFα and the surface markers CCR7, CD80, and HLADR/MHC Class II; M2a macrophages upregulated cytokines CCL18 and MDC/CCL22 and the surface marker CD206/mannose receptor; M2c macrophages unregulated the scavenger receptor CD163 (RT-PCR using monocytes/macrophages from n=9 human donors). FIG. 1C shows all macrophages expressed similar levels of CCR7, CD206, CD163, HLADR in terms of the number of positive cells, whereas FIG. 1D shows mean fluorescent intensity per cell indicated greater differences in expression of surface markers: CCR7 for M1, CD206 for M2a, and CD163 for M2c (flow analysis using cells from n=3-5 human donors). Data are shown as Mean±SEM. *p<0.05, **p<0.01, ***p<0.001.

FIG. 2 is a series of bar graphs and a gel image showing gene expression and protein secretion levels imply phenotypically dependent roles of macrophages in angiogenesis. In FIG. 2A, upregulated expression of VEGF, bFGF, IL8, and RANTES/CCL5 suggest M1 involvement during the early stages of angiogenesis. Expression of HBEGF by M1 and PDGFB by M2a suggest involvement during later stages when recruiting cells responsible for stabilization of neovasculature is essential. M2a expression of angiogenic inhibitor TIMP3 suggests a regulatory role in the process (n=10-11 human donors). In FIG. 2B, ELISA of macrophage-conditioned media confirmed significantly higher levels of protein secretion of VEGF by M1, PDGF-BB by M2a, and MMP-9 by both M0 and M2c. MMP-9 secretion was significantly decreased by M2a (n=4-6 human donors). In FIG. 2C, enzymatic activity for MMP-9 was confirmed by gel zymography (representative gel shown, n=5 human donors). Data are presented as Mean±SEM.*p<0.05, **p<0.01, ***p<0.001.

FIG. 3 is a series of images, bar graphs, and a cartoon showing functionality of macrophage-secreted factors in angiogenesis. In FIG. 3A, an in vitro sprouting assay was used to assess HUVEC organization on Matrigel® in macrophage-conditioned media. Networks were analyzed using the Angiogenesis Analyzer macro in ImageJ following background subtraction in MATLAB. Vascular networks in M2c-conditioned media contained significantly more sprouts and were of greater total length than those in media conditioned by M1 or M2a macrophages and the negative control (RPMI media with 10% heat-inactivated human serum). Networks formed in M2a-conditioned media were not statistically different than those in the negative control. Data are shown as Mean±SEM(3-5). ⁰ Non-significant differences compared to control group (RPMI media only); ^{#} Significantly different from control group with p<0.05, *p<0.05, **p<0.01, ***p<0.001. In FIG. 3B, collectively, the phenotypic characterization and functional assays of macrophages suggest that all three macrophage phenotypes function together in angiogenesis: M1 macrophages recruit endothelial cells and initiate angiogenesis via secretion of VEGF, M2a macrophages recruit stabilizing pericytes via PDGF-B and regulate VEGF signaling and MMP-9 activity via TIMP3, and M2c macrophages permit matrix remodeling and blood vessel growth via MMP-9.

FIG. 4 is a series of images showing relationships between macrophage phenotype and scaffold vascularization in vivo after 10 days in a subcutaneous implantation model in mice. In FIG. 4A, modifications to collagen scaffolds revealed markedly different outcomes upon gross inspection. Unmodified collagen scaffolds remained avascular and were encapsulated in a dense fibrous capsule. In contrast, glutaraldehyde-crosslinked scaffolds appeared well integrated and vascularized, with macroscopically detectable robust infiltration of blood vessels. LPS-coated scaffolds were infiltrated by inflammatory tissue. The unmodified and LPS-coated scaffolds were both considerably smaller and more degraded than crosslinked scaffolds (n=4-6). Scale bar: 2 mm. In FIG. 4B, scaffolds and surrounding tissue were stained with H&E and for the endothelial cell marker CD31 (lower left inset). In contrast to the glutaraldehyde-crosslinked sections, which had many blood vessels that stained positively for CD31, no blood vessels were observed in either the unmodified or the LPS-coated scaffolds. LPS-coated scaffolds were completely infiltrated by inflammatory cells (n=4-6). Scale bar: 100 μm. In FIG. 4C, FIG. 4D, FIG. 4E, and FIG. 4F, sections of explanted scaffolds with surrounding tissue were stained for multiple markers of M1 and M2 macrophage phenotypes in combination with the pan-macrophage marker F480. Both the glutaraldehyde-crosslinked and LPS-coated scaffolds were infiltrated by F480⁺ macrophages, while the unmodified collagen scaffolds, encased in a fibrous capsule, showed macrophage localization on the outside only. Macrophages surrounding unmodified collagen scaffolds stained weakly for the M1 markers and strongly for the M2 markers. Glutaraldehyde-crosslinked scaffolds stained strongly for all M1 and M2 markers examined except Arg1, which was not detected. LPS-coated scaffolds stained strongly for the M1 markers and weakly for the M2 markers. CD206 and CCR7 expression did not differ between groups (n=4-6). Scale bar: 100 μm.

FIG. 5 is a series of graphs showing flow cytometric analysis of macrophage surface markers for phenotypic characterization. FIG. 5A shows double stains and FIG. 5B shows single stains.

FIG. 6 is a series of bar graphs showing 18-hour HUVEC viability and metabolic assays for n=9 technical replicates. No significant differences were observed. Data is represented as mean+SEM.

FIG. 7 is a series of images showing negative control images (deleted primary antibody) for immunofluorescent staining in FIG. 4C-F.

FIG. 8 is a series of images showing image processing for in vitro sprout formation assay depicted in FIG. 3.

FIG. 9 is a cartoon, a series of bar graphs, and a series of images showing that scaffolds that promote the M1 phenotype of macrophages followed by the M2 phenotype can increase vascularization. In FIG. 9A, macrophages are differentiated from monocytes and polarized to different phenotypes. In FIG. 9B, M1 macrophages express and secrete growth factors important in early stages of angiogenesis, while M2 macrophages express and secrete growth factors important in later stages of angiogenesis. In FIG. 9C, endothelial cells increase sprout formation in M0 and M1-conditioned media, but not M2-conditioned media (see also FIG. 8). In FIG. 9D, macrophages can switch their phenotype from M1 to M2 or vice versa. In FIG. 9E, both M1 and M2 macrophages are required for scaffold vascularization. In FIG. 9F, scaffolds with conjugated IL4 can cause M2 polarization of seeded macrophages.

FIG. 10 is a pair of images with a cartoon overlay showing a scaffold with attached IL4 and physically adsorbed IFNy, which would be cleared relatively quickly (˜1 day) from the scaffolds, thus promoting the M1 response followed by the M2 response. In FIG. 10A, adsorbed IFNy causes macrophages in the vicinity to polarize to the M1 phenotype. They release angiogenic growth factors such as VEGF, recruit endothelial cells, and initiate the process of angiogenesis. In FIG. 10B, when the adsorbed IFNy is cleared, the IL4 attached to the scaffold becomes exposed. M1 macrophages convert to the M2 phenotype and secrete factors such as PDGF that recruit pericytes to stabilize the growing vasculature.

FIG. 11 is a cartoon showing the ability of polarized macrophages to switch phenotypes. Monocyte-derived macrophages were exposed to M1- or M2-polarizing stimuli for 3 days followed by polarizing stimuli of the other phenotype for an additional 3 days (M1→M2 and M2→M1). Unstimulated macrophages (M0) or macrophages cultured under M1- or M2-polarizing stimuli for 6 days (M1 and M2), with a media change at day 3, served as controls. Additional information regarding methodology is provided in Example 8.

FIG. 12 is a series of cartoons showing ability of scaffolds to facilitate phenotypic switch. FIG. 12A shows scaffolds with physically adsorbed IFN-gamma are expected to cause initial polarization of macrophages to the M1 phenotype. M1 macrophages release angiogenic growth factors such as VEGF, recruit endothelial cells, and initiate the process of angiogenesis. Scaffolds would then release IL4, which would convert M1 macrophages to the M2 phenotype. M2 macrophages secrete factors such as PDGF-BB that recruit pericytes to stabilize the growing vasculature. FIG. 12B shows a small molecule biotin is covalently conjugated to the scaffolds and to IL4, preserving their bioactivity and allowing them to be joined to the scaffolds using streptavidin. Additional information regarding methodology is provided in Example 8.

FIG. 13 is a series of plots showing kinetics of macrophage phenotype switching. FIG. 13A shows mean intensity of expression of CCR7 and CD206 per cell on day 4 and day 6 determined by flow cytometry. FIG. 13B shows percent of population of cells as a function of time in days. Additional information regarding methodology is provided in Example 8.

FIG. 14 is a series of line plots showing temporal changes in macrophage gene expression. Data are shown as fold change over M0 controls at the same time point. Markers in the left columns are M1 markers (FIG. 14A, TNFa; FIG. 14C, IL1 b; FIG. 14E, CCR7; FIG. 14G, VEGF), while markers in the right column are M2 markers (FIG. 14B, CCL18; FIG. 14D, MDC; FIG. 14F, CD206; FIG. 14H, PDGF; FIG. 14I, TIMP3). Gene expression was analyzed by RT-PCR after 1, 3, 4, and 6 days of culture. Lines connecting data points are used to show relationships between time points and do not indicate a linear relationship. Additional information regarding methodology is provided in Example 8.

FIG. 15 is a series of line plots showing changes in cytokine secretion over time. Markers in the left columns are M1 markers (FIG. 15A, TNFa; FIG. 15C, VEGF), while markers in the right column are M2 markers (FIG. 15B, CCL18; FIG. 15D, PDGF-BB). Data are shown for cell culture media after 1, 2, 3, 4, and 6 days, as assessed by ELISA. Additional information regarding methodology is provided in Example 8.

FIG. 16 is a series of images and scatter plots showing an immunomodulatory scaffold. FIG. 16A shows fluorescent streptavidin bound to biotinylated scaffolds but not to non-biotinylated scaffolds (FIG. 16B), assessed using confocal microscopy. FIG. 16C shows cumulative release of IFN-gamma from IFNg scaffolds. FIG. 16D shows cumulative release of IL4 from IL4 scaffolds. Additional information regarding methodology is provided in Example 9.

FIG. 17 is a series of bar graphs showing time changes in gene expression in macrophages on immunomodulatory scaffolds. Representative data are shown from experiments that were repeated three times. Markers in the left columns are M1 markers (FIG. 14A, TNFa; FIG. 14C, IL1 b; FIG. 17E, CCR7; FIG. 17G, VEGF), while markers in the right column are M2 markers (FIG. 17B, CCL18; FIG. 17D, MDC; FIG. 17F, CD206; FIG. 17H, PDGF; FIG. 17I, TIMP3). Gene expression levels were compared to the negative control and analyzed using one-way ANOVA at each time point with Dunnett's post-hoc analysis (mean±SEM, n=4; *p<0.05, **p<0.01, and ***p<0.001). Additional information regarding methodology is provided in Example 9.

FIG. 18 is a series of bar graphs showing cytokine secretion by macrophages seeded on scaffolds. *p<0.05 by one-way ANOVA and Tukey's post-hoc analysis; #p<0.01 and ***p<0.001 by one-way ANOVA followed by Dunnett's post hoc analysis (mean±SEM, n=5). Markers in the left columns are M1 markers (FIG. 18A, TNFa; FIG. 18C, VEGF), while markers in the right column are M2 markers (FIG. 18B, CCL18; FIG. 18D, PDGF-BB). Additional information regarding methodology is provided in Example 9.

FIG. 19 is a series of images and a bar graph showing scaffolds after 2 weeks of subcutaneous implantation in mice. FIG. 19A, gross view; FIG. 19B, H&E staining; FIG. 19C, immunohistochemical analysis for the endothelial cell marker CD31 (green) and counterstained with DAPI (blue); FIG. 19D, delete primary control; and FIG. 19E, quantification of CD31 intensity after subtracting values of intensity of the delete primary controls (mean±SEM, n=3). Additional information regarding methodology is provided in Example 10.

### DETAILED DESCRIPTION OF THE INVENTION

The present disclosure is based, at least in part, on the discovery that biomaterials with attached cytokines can direct the macrophage phenotype upon implantation into the body, which is a critical determinant of the success or failure of a biomaterial. As shown herein, in contrast to the traditionally understood paradigm, primary human M1 macrophages can secrete highly elevated levels of potent angiogenic stimulators including VEGF; M2a macrophages secrete highly elevated levels of PDGF-BB, a chemoattractant for stabilizing pericytes; and M2c macrophages secrete highly elevated levels of MMP9, an important protease involved in remodeling. Furthermore, sequential promotion of an M1 macrophage phenotype followed by promotion of an M2 macrophage phenotype can increase vascularization of a scaffold.

It is presently believed that both M1 and M2 macrophages are required for scaffold vascularization, and if the balance of macrophage phenotype is pushed too far to either extreme of the M1 to M2 spectrum, then vascularization and integration may not be achieved.

Findings described herein are in contrast to the conventional understanding that macrophages exist on a spectrum of phenotypes ranging from M1, believed to be solely pro-inflammatory, to M2, believed to promote angiogenesis, tissue repair, and scaffold vascularization. It is demonstrated herein that M1 macrophages secrete growth factors that can be potent stimulators of early angiogenesis, while two different subsets of M2 macrophages secrete factors that can be involved in later stages of angiogenesis. It is presently believed that both M1 and M2 macrophages are required for scaffold vascularization, and that temporal control over the macrophage response can be utilized to enhance vascularization.

Thus is provided a novel regenerative approach for tissue defects from synergistic actions of both M1 and M2 macrophages, such that the total effect can be greater than the sum of the individual effects. Such approaches benefit from the new understanding, disclosed herein, of the temporal or spatial interactions between M1 and M2 macrophages, and their cell lineage derivatives with regulatory growth factors in the de novo formation of vascularized tissues or organs.

For example, a scaffold can include a biocompatible matrix, growth factor IL4 attached to the matrix, and growth factor IFNy physically adsorbed onto or into the matrix. The matrix can be engineered such that the adsorbed IFNy is cleared or substantially cleared within a period of time (e.g., about one day). The initial presence of IFNy can promote an M1 response and when the IFNy clears or substantially clears, the IL4 can promote an M2 response.

Another aspect of the invention provides methods for the formation of engineered vascularized tissue or organ from such constructs. In various embodiments, compositions (e.g., compositions comprising one or more growth factors) that promote an M1, M2A or M2C phenotype can be introduced into or onto a biocompatible matrix. Such a matrix can provide a scaffold for production of a vascularized tissue or organ. A further aspect provides a method of treating a tissue defect by grafting a composition of the present disclosure into a subject in need thereof.

Macrophage Phenotype

It is demonstrated herein that M1 macrophages secrete growth factors that can be potent stimulators of early angiogenesis, while two different subsets of M2 macrophages secrete factors that can be involved in later stages of angiogenesis. Specifically, M1 macrophages can secrete highly elevated levels of potent angiogenic stimulators including VEGF; M2a macrophages secrete highly elevated levels of PDGF-BB, a chemoattractant for stabilizing pericytes; and M2c macrophages secrete highly elevated levels of MMP9, an important protease involved in remodeling. Thus, both M1 and M2 macrophages are required for scaffold vascularization, and temporal control over macrophage response can be utilized to enhance vascularization.

Working examples demonstrate that M1 macrophages secrete more angiogenic factors including VEGF than M2 macrophages, which have been conventionally referred to as the angiogenic phenotype (see e.g., Mantovani et al. 2004 Trends Immunol 25(12), 677-686), even though VEGF secretion has been linked to M1 polarization before (see e.g., Kiriakidis et al. 2003 Journal of Cell Science 116(Pt 4), 665-674). In the context of biomaterial vascularization, M2 macrophages may be more angiogenic in vivo because of their role in recruiting stabilizing pericytes. Another possibility is that the angiogenic behavior of M2-like tumor-associated macrophages has been attributed to M2 macrophages in other contexts. But M2a and M2c macrophages, often lumped together, behave very differently in the context of angiogenesis.

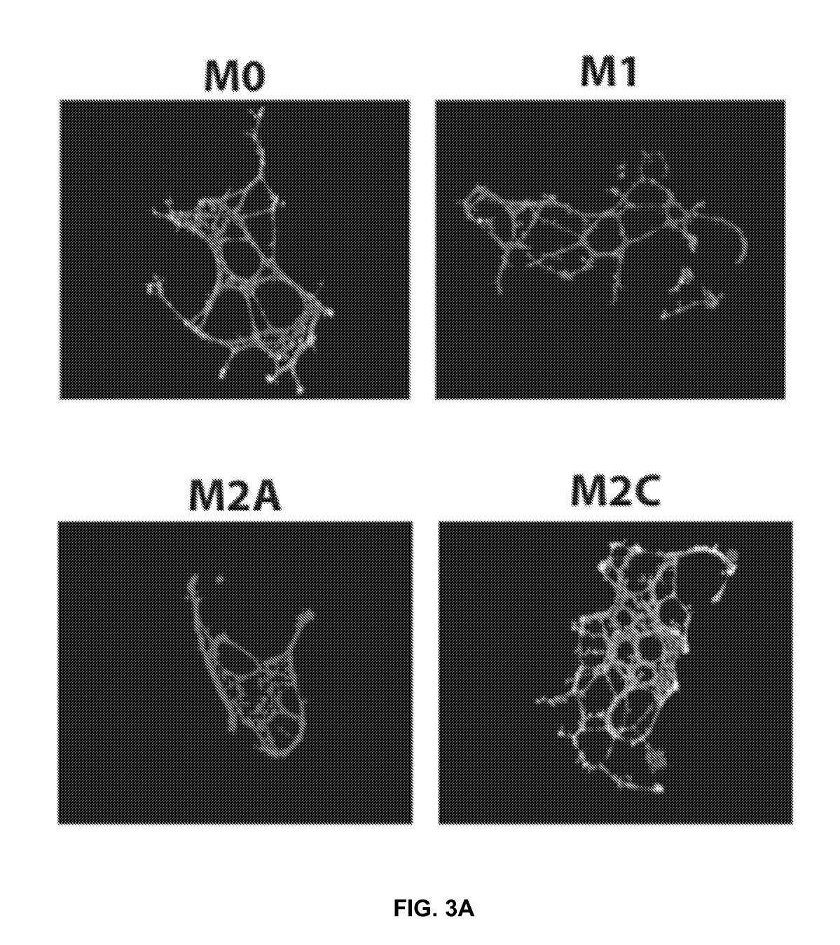

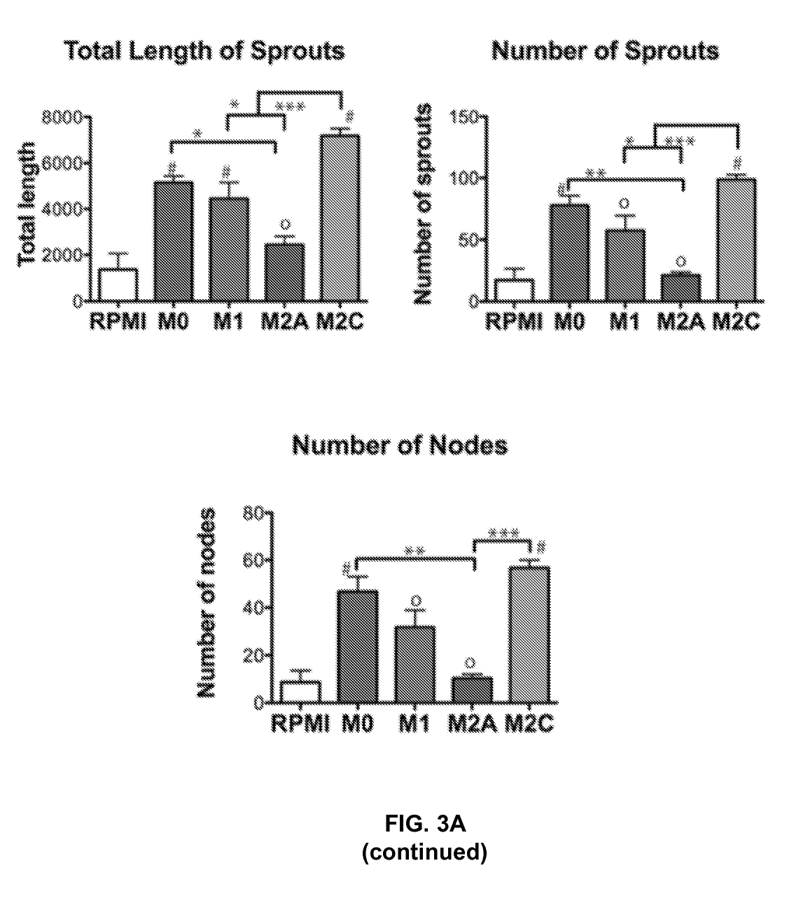

Results described herein support that all three macrophage phenotypes function together in angiogenesis: M1 macrophages recruit endothelial cells and initiate angiogenesis via secretion of VEGF, M2a macrophages recruit stabilizing pericytes via PDGF-B and regulate VEGF signaling and MMP-9 activity via TIMP3, and M2c macrophages permit matrix remodeling and blood vessel growth via MMP-9 (see e.g., FIG. 3B).

Both M1 and M2 macrophage phenotype actions can be applied in a temporally precise way that can provide for proper vascularization. Specifically, M1 macrophages can initiate a process of angiogenesis by secreting VEGF, a potent chemoattractant for endothelial cells. Next, M2a macrophages can negatively regulate the actions of M1 macrophages by blocking TNFα and VEGF via TIMP3 secretion, and also secrete PDGF-B in order to recruit pericytes to stabilize the growing vasculature. Further, M2c macrophages can secrete MMP9 and therefore play a role in tissue remodeling for new blood vessel formation.

Sequential promotion of an M1 macrophage phenotype followed by promotion of an M2 macrophage phenotype can increase vascularization of a scaffold. Exposure to only M1 cytokines has been shown to cause inflammation (see e.g., Spiller et al. 2014 Biomaterials 35, 4477-4488). Exposure to only M2 cytokines has been shown to cause fibrous encapsulation of the implanted material (see e.g., Spiller et al. 2014 Biomaterials 35, 4477-4488). As shown herein, combined exposure (e.g., sequential exposure) to M1 macrophages and M2 macrophages can result in vascularization.

As shown herein, in an in vivo subcutaneous implantation model, porous collagen scaffolds were surrounded by a fibrous capsule, coincident with the highest numbers of M2 macrophages; scaffolds coated with the bacterial lipopolysaccharide were degraded by inflammatory macrophages; and crosslinked collagen scaffolds were infiltrated by substantial numbers of blood vessels, accompanied by high levels of both M1 and M2 macrophages. These results support that both M1 and M2 macrophages are required for scaffold vascularization, and that temporal control over the macrophage response can be utilized to enhance vascularization.

Furthermore, it is determined herein that attachment of interferon-gamma (IFNy) can be used to generate the M1 phenotype of macrophages, interleukin-4 (IL4) to generate the M2A phenotype of macrophages, and IL10 to generate M2C macrophages.

In various embodiments, scaffold vascularization can be achieved by modifying scaffold properties to control the inflammatory response. Both M1 and M2 macrophages can be used to achieve vascularization; scaffolds with a primarily M2 response were shown to be surrounded by a fibrous capsule, and those with a primarily M1 response were characterized by infiltrating inflammatory cells. Regarding M1 macrophages, this study and other studies (see e.g., Tous et al. 2012 Acta Biomaterialia 8(9), 3218-3227; Bota et al. 2010 Journal of Biomedical Materials Research, Part A 95(2), 649-657; Tolg et al. 2012 The American Journal of Pathology 181(4), 1250-1270) showed that M1 macrophages are beneficial for scaffold vascularization in vivo. It is presently believed that both M1 and M2 macrophages are required for scaffold vascularization, and if the balance of macrophage phenotype is pushed too far to either extreme of the M1 to M2 spectrum, then vascularization and integration may not be achieved.

Thus, four macrophage phenotypes have been systematically characterized in the context of angiogenesis and evidence provided that all four phenotypes are beneficial to angiogenesis, and for different reasons. M1 macrophages secreted high levels of the potent angiogenic factor VEGF. For at least these reasons, M2 macrophages should no longer be considered as a sole angiogenic phenotype (as is conventionally understood). By modifying scaffold properties to control the macrophage response, one can achieve robust scaffold vascularization. Tissue engineering strategies that incorporate knowledge of macrophage behavior can result in control over vascularization or integration, which can play an important role in clinical translation of tissue-engineering strategies.

An M1 macrophage can include one or more of the following markers: TNFα, IL1b, CCR7, or VEGF (see e.g., Example 1, Example 8). An M2 macrophage can include one or more of the following markers: CCL18, MDC, CD206, PDGF, or TIMP3 (see e.g., Example 1, Example 8).

Tissue

Biologically viable tissue or organ can be engineered from a scaffold described herein with improved vascularization through the use of temporal or spatial interactions between M1 and M2 macrophages. Vascularized tissue or organ types that can be formed according to the methods described herein include, but are not limited to, bladder, bone, brain, breast, osteochondral junction, nervous tissue including central nervous system, spinal cord and peripheral nerve, glia, esophagus, fallopian tube, heart, pancreas, intestines, gallbladder, kidney, liver, lung, ovaries, prostate, spinal cord, spleen, skeletal muscle, skin, stomach, testes, thymus, thyroid, trachea, urogenital tract, ureter, urethra, interstitial soft tissue, periosteium, periodontal tissue, cranial sutures, hair follicles, oral mucosa, or uterus. For example, a soft tissue composition can be vascularized adipose tissue. As another example, a hard tissue composition can be vascularized bone tissue.

A tissue is generally understood to be a collection of cells having a similar morphology and function, and frequently supported by heterogenous interstitial tissues with multiple cell types and blood supply. An organ is generally a collection of tissues that perform a biological function. Organs can be, but are not limited to, bladder, brain, nervous tissue, glial tissue, esophagus, fallopian tube, bone, synovial joint, cranial sutures, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage. The biological function of an organ can be assayed using standard methods known to the skilled artisan.

Vascularization

Promotion of M1 macrophage or M2 macrophage phenotypes via growth factors in or on a matrix material of a scaffold can increase vascularization of the scaffold. Blood vessels can grow throughout the scaffold so as to form a engineered vascularized tissue or organ. Vascularization can be produced in the engineered tissue or organ in vitro, in vivo, or a combination thereof. For example, differentiation can be carried out by culturing progenitor cells in the matrix material of the scaffold. As another example, progenitor cells can be infused into the matrix, and such matrix promptly engrafted into a subject, allowing differentiation to occur in vivo. The determination of when to introduce the engineered tissue or organ into a subject can be based, at least in part, on the amount of vascularization formed in the tissue or organ.

Methods for measuring angiogenesis in the engineered tissue or organ are standard in the art (see e.g., Jain et al. (2002) Nat. Rev. Cancer 2:266-276; Ferrara, ed. (2006) Angiogenesis, CRC, ISBN 0849328446). During early blood vessel formation, immature vessels resemble the vascular plexus during development, by having relatively large diameters and lacking morphological vessel differentiation. Over time, the mesh-like pattern of immature angiogenic vessels gradually mature into functional microcirculatory units, which develop into a dense capillary network having differentiated arterioles and venules. Angiogenesis can be assayed, for example, by measuring the number of non-branching blood vessel segments (number of segments per unit area), the functional vascular density (total length of perfused blood vessel per unit area), the vessel diameter, or the vessel volume density (total of calculated blood vessel volume based on length and diameter of each segment per unit area).

Scaffolds described herein generally provide for increased vascularization as compared to engineered tissue or organ produced according to conventional means. For example, blood vessel formation (e.g., angiogenesis, vasculogenesis, formation of an immature blood vessel network, blood vessel remodeling, blood vessel stabilization, blood vessel maturation, blood vessel differentiation, or establishment of a functional blood vessel network) in an engineered tissue or organ can be increased by at least 5%, 10%, 20%, 25%, 30%, 40%, or 50%, 60%, 70%, 80%, 90%, or even by as much as 100%, 150%, or 200%, or more, compared to a corresponding engineered tissue or organ that is not formed by promoting M1 macrophage and M2 macrophage phenotypes as described herein. The vascularization of an engineered tissue or organ composition can be a stable network of blood vessels that endures for at least 1 day, 2 days, 3 days, 4 days, 5 days, 6 days, 1 week, 2 weeks, 3 weeks, 1 month, 2 months, 3 months, 4 months, 5 months, 6 months, or 12 months or more. A vascular network of the engineered tissue or organ composition can be integrated into the circulatory system of the tissue, organ, or subject upon introduction thereto.

For tissue or organ regeneration using small scaffolds (<100 cubic millimeters in size), in vitro medium can be changed manually, and additional agents added periodically (e.g., every 3-4 days). For larger scaffolds, the culture can be maintained, for example, in a bioreactor system, which may use a minipump for medium change. The minipump can be housed in an incubator, with fresh medium pumped to the matrix material of the scaffold. The medium circulated back to, and through, the matrix can have about 1% to about 100% fresh medium. The pump rate can be adjusted for optimal distribution of medium or additional agents included in the medium. The medium delivery system can be tailored to the type of tissue or organ being manufactured. All culturing can be performed under sterile conditions.

Scaffold

A scaffold described herein can have immunomodulatory activity sufficient to enhance vascularization through the action of host macrophages while minimizing disruptive effects on osteogenic properties.

As described herein, a scaffold containing one or more compositions that promote an M1 macrophage or an M2 macrophage phenotype has a higher potential for vascularization when cultured with cells or implanted in a subject. For example, compositions and methods of the present disclosure can employ a scaffold, into or onto which compositions that promote M1 macrophage or M2 macrophage phenotype can be introduced so as to promote vascularization of an engineered tissue or organ construct.

One aspect of the present disclosure provides for tissue scaffolds or coated or filled biomaterial compositions. A scaffold described herein can promote vascularization or healing by first promoting an M1 response followed by the M2a or a M2c response. Compositions can include scaffolds that can promote an M1, M2A or M2C phenotype via attachment of growth factors, such as IFNy, LPS, TNFα, IL4 or IL10.

In some embodiments, the scaffold can form a structure for growth or regeneration of a tissue. In other embodiments, a scaffold can compose or be incorporated in or on an implanted biomaterial. Accordingly, modification of an implanted biomaterial (e.g., joint replacement materials, stents, pacemakers, etc.) according to an methods or materials described herein can result in better healing and integration.

In some embodiments, a scaffold includes a cell, for example a progenitor cell (e.g., a transplanted mammalian progenitor cell). In other embodiments, a scaffold is cell-free until it is implanted in a subject, i.e., no cell is applied to the scaffold; any cell present in the scaffold migrated into the scaffold.

A scaffold can be composed in whole or in part by a matrix material. A scaffold can be fabricated with any matrix material recognized as useful by the skilled artisan. A matrix material can be a biocompatible material that generally forms a porous, microcellular scaffold, which provides a physical support for cells migrating thereto. Such matrix materials can: allow cell attachment and migration; deliver and retain cells and biochemical factors; enable diffusion of cell nutrients and expressed products; or exert certain mechanical and biological influences to modify the behavior of the cell phase. The matrix material generally forms a porous, microcellular scaffold of a biocompatible material that provides a physical support and an adhesive substrate for recruitment and growth of cells during in vitro or in vivo culturing.

Suitable scaffold and matrix materials are discussed in, for example, Ma and Elisseeff, ed. (2005) Scaffolding In Tissue Engineering, CRC, ISBN 1574445219; Saltzman (2004) Tissue Engineering: Engineering Principles for the Design of Replacement Organs and Tissues, Oxford ISBN 019514130X. For example, matrix materials can be, at least in part, solid xenogenic (e.g., hydroxyapatite) (Kuboki et al. 1995 Connect Tissue Res 32, 219-226; Murata et al. 1998 Int J Oral Maxillofac Surg 27, 391-396), solid alloplastic (polyethylene polymers) materials (Saito and Takaoka 2003 Biomaterials 24 2287-93; Isobe et al. 1999 J Oral Maxillofac Surg 57, 695-8), or gels of autogenous (Sweeney et al. 1995. J Neurosurg 83, 710-715), allogenic (Bax et al. 1999 Calcif Tissue Int 65, 83-89; Viljanen et al. 1997 Int J Oral Maxillofac Surg 26, 389-393), or alloplastic origin (Santos et al. 1998. J Biomed Mater Res 41, 87-94), and combinations of the above (Alpaslan et al. 1996 Br J of Oral Maxillofac Surg 34, 414-418).

A matrix configuration can be dependent on a tissue or organ that is to be repaired or produced, but generally the matrix can be a pliable, biocompatible, porous template that allows for vascular and target tissue or organ growth. A matrix can be fabricated into structural supports, where the geometry of the structure (e.g., shape, size, porosity, micro- or macro-channels) can be tailored to the application. The porosity of the matrix can be a design parameter that influences cell introduction or cell infiltration. The matrix can be designed to incorporate extracellular matrix proteins that influence cell adhesion and migration in the matrix.

A matrix material can have an adequate porosity or an adequate pore size so as to facilitate cell recruitment and diffusion throughout the whole structure of both cells and nutrients. A matrix can be biodegradable providing for absorption of the matrix by the surrounding tissues, which can eliminate the necessity of a surgical removal. The rate at which degradation occurs can coincide as much as possible with the rate of tissue or organ formation. Thus, while cells are fabricating their own natural structure around themselves, the matrix is able to provide structural integrity and eventually break down, leaving the neotissue, newly formed tissue or organ which can assume the mechanical load. The matrix can be an injectable matrix in some configurations. The matrix can be delivered to a tissue using minimally invasive endoscopic procedures.

A scaffold can comprise a matrix material having different phases of viscosity. For example, a matrix can have a substantially liquid phase or a substantially gelled phase. The transition between phases can be stimulated by a variety of factors including, but limited to, light, chemical, magnetic, electrical, and mechanical stimulus. For example, the matrix can be a thermosensitive matrix with a substantially liquid phase at about room temperature and a substantially gelled phase at about body temperature. The liquid phase of the matrix can have a lower viscosity that provides for optimal distribution of growth factors or other additives and injectability, while the solid phase of the matrix can have an elevated viscosity that provides for matrix retention at or within the target tissue.

The scaffold can comprise a matrix material formed of synthetic polymers. Such synthetic polymers include, but are not limited to, polyurethanes, polyorthoesters, polyvinyl alcohol, polyamides, polycarbonates, polyvinyl pyrrolidone, marine adhesive proteins, cyanoacrylates, analogs, mixtures, combinations and derivatives of the above. Alternatively, the matrix can be formed of naturally occurring biopolymers. Such naturally occurring biopolymers include, but are not limited to, fibrin, fibrinogen, fibronectin, collagen, and other suitable biopolymers. Also, the matrix can be formed from a mixture of naturally occurring biopolymers and synthetic polymers.

The matrix can include naturally occurring polymers or natively derived polymers. Such polymers include, but are not limited to, agarose, alginate, fibrin, fibrinogen, fibronectin, collagen, gelatin, hyaluronic acid, and other suitable polymers and biopolymers, or analogs, mixtures, combinations, and derivatives of the above. Also, the matrix can be formed from a mixture of naturally occurring biopolymers and synthetic polymers.

A matrix material can include, for example, a collagen gel, a polyvinyl alcohol sponge, a poly(D,L-lactide-co-glycolide) fiber matrix, a polyglactin fiber, a calcium alginate gel, a polyglycolic acid mesh, polyester (e.g., poly-(L-lactic acid) or a polyanhydride), a polysaccharide (e.g. alginate), polyphosphazene, or polyacrylate, or a polyethylene oxide-polypropylene glycol block copolymer. Matrices can be produced from proteins (e.g. extracellular matrix proteins such as fibrin, collagen, and fibronectin), polymers (e.g., polyvinylpyrrolidone), or hyaluronic acid. Synthetic polymers can also be used, including bioerodible polymers (e.g., poly(lactide), poly(glycolic acid), poly(lactide-co-glycolide), poly(caprolactone), polycarbonates, polyamides, polyanhydrides, polyamino acids, polyortho esters, polyacetals, polycyanoacrylates), degradable polyurethanes, non-erodible polymers (e.g., polyacrylates, ethylene-vinyl acetate polymers and other acyl substituted cellulose acetates and derivatives thereof), non-erodible polyurethanes, polystyrenes, polyvinyl chloride, polyvinyl fluoride, poly(vinylimidazole), chlorosulphonated polyolifins, polyethylene oxide, polyvinyl alcohol, Teflon®, or nylon.

A matrix can include one or more of enzymes, ions, growth factors, or biologic agents. For example, the matrix can contain a growth factor (e.g., a growth factor that promotes an M1 macrophage or an M2 macrophage phenotype, an angiogenic growth factor, or a tissue specific growth factor). Such a growth factor can be supplied at a concentration of about 0 to 1000 ng/mL. For example, the growth factor can be present at a concentration of about 100 to 700 ng/mL, at a concentration of about 200 to 400 ng/mL, or at a concentration of about 250 ng/mL.

The concentration of a compound or a composition in the scaffold will vary with the nature of the compound or composition, its physiological role, and desired therapeutic or diagnostic effect. A therapeutically effective amount is generally a sufficient concentration of therapeutic agent to display the desired effect without undue toxicity. The compound can be incorporated into the scaffold or matrix material by any known method.

Chemical modification methods can be used to covalently link a compound or a composition to a matrix material. The surface functional groups of the matrix can be coupled with reactive functional groups of a compound or a composition to form covalent bonds using coupling agents well known in the art such as aldehyde compounds, carbodiimides, and the like. Additionally, a spacer molecule can be used to gap the surface reactive groups and the reactive groups of the biomolecules to allow more flexibility of such molecules on the surface of the matrix. Other similar methods of attaching biomolecules to the interior or exterior of a matrix will be known to one of skill in the art.

Biomolecules

It has been shown that attachment of interferon-gamma (IFNy) can be used to generate the M1 phenotype of macrophages, interleukin-4 (IL4) to generate the M2A phenotype of macrophages, and IL10 to generate M2C macrophages.

Various embodiments provide for methods or compositions to control of the inflammatory response to a biomaterial for a beneficial effect on healing and integration with the body. Accordingly, modification of an implanted biomaterial (e.g., joint replacement materials, stents, pacemakers, etc.) according to methods or materials described herein can result in better healing and integration.

Interferon-Gamma.

Interferon-gamma (IFNy) can promote the M1 phenotype of macrophages. IFNy is the sole member of Type II class of interferons and is a member of the larger family of macrophage-activating factor proteins. In humans, IFNy is encoded by the IFNG gene. The sequence and structure of IFNy is well characterized in the art. Cellular responses to IFNy are activated through its interaction with a heterodimeric receptor consisting of Interferon gamma receptor 1 (IFNGR1) and Interferon gamma receptor 2 (IFNGR2), where binding thereto activates the JAK-STAT pathway. IFNy also binds to the glycosaminoglycan heparan sulfate (HS) at the cell surface, which inhibits HS. IFNy is commercially available (see e.g., R&D Systems, Minneapolis, Minn.; Actimmune®, Vidara Therapeutics, Roswell, Ga.). IFNy can be formulated as generally described herein.

In some embodiments, IFNy can be attached (e.g., covalently bound) in or on a scaffold. IFNy can be present in or on a scaffold at a concentration of about 0.1 ng/mm³ to about 125 ng/mm³. For example, IFNy can be present in or on a scaffold at a concentration of about 0.1 ng/mm³, about 1 ng/mm³, about 2 ng/mm³, about 3 ng/mm³, about 4 ng/mm³, about 5 ng/mm³, about 6 ng/mm³, about 7 ng/mm³, about 8 ng/mm³, about 9 ng/mm³, about 10 ng/mm³, about 11 ng/mm³ about 12 ng/mm³, about 13 ng/mm³, about 14 ng/mm³, about 15 ng/mm³ about 16 ng/mm³, about 17 ng/mm³, about 18 ng/mm³, about 19 ng/mm³ about 20 ng/mm³, about 25 ng/mm³, about 30 ng/mm³, about 35 ng/mm³ about 40 ng/mm³, about 45 ng/mm³, about 50 ng/mm³, about 55 ng/mm³ about 60 ng/mm³, about 65 ng/mm³, about 70 ng/mm³, about 75 ng/mm³ about 80 ng/mm³, about 85 ng/mm³, about 90 ng/mm³, about 95 ng/mm³ about 100 ng/mm³, about 105 ng/mm³, about 110 ng/mm³, about 115 ng/mm³ about 120 ng/mm³, about 125 ng/mm³, or more. As another example, a fluid medium adsorbed into a scaffold can contain IFNy a concentration of about 12.5 ng/mm³. It is understood that recitation of the above discrete values includes a range between each recited value.

In some embodiments, a fluid medium containing IFNy is adsorbed into a scaffold. A fluid medium adsorbed into a scaffold can contain IFNy at a concentration of about 10 ng/ml to about 1,000 ng/ml. For example, a fluid medium adsorbed into a scaffold can contain IFNy at a concentration of about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, about 150 ng/ml, about 160 ng/ml, about 170 ng/ml, about 180 ng/ml, about 190 ng/ml, about 200 ng/ml, about 250 ng/ml, about 300 ng/ml, about 350 ng/ml, about 400 ng/ml, about 450 ng/ml, about 500 ng/ml, about 550 ng/ml, about 600 ng/ml, about 650 ng/ml, about 700 ng/ml, about 750 ng/ml, about 800 ng/ml, about 850 ng/ml, about 900 ng/ml, about 950 ng/ml, about 1,000 ng/ml, or more. As another example, a fluid medium adsorbed into a scaffold can contain IFNy at a concentration of about 100 ng/ml. It is understood that recitation of the above discrete values includes a range between each recited value.

Lipopolysaccharide.

Lipopolysaccharide (LPS), also known as lipoglycans, can promote the M1 phenotype of macrophages. LPS are large molecules having a lipid and a polysaccharide joined by a covalent bond. LPS is a component of the outer membrane of Gram-negative bacteria, can act as endotoxins, and can elicit strong immune responses in animals. LPS is commercially available (see e.g., Sigma-Aldrich, St. Louis, Mo.). LPS can be formulated as generally described herein.

In some embodiments, LPS can be attached (e.g., covalently bound) in or on a scaffold. LPS can be present in or on a scaffold at a concentration of about 0.1 ng/mm³ to about 125 ng/mm³. For example, LPS can be present in or on a scaffold at a concentration of about 0.1 ng/mm³, about 1 ng/mm³, about 2 ng/mm³, about 3 ng/mm³, about 4 ng/mm³, about 5 ng/mm³, about 6 ng/mm³, about 7 ng/mm³, about 8 ng/mm³, about 9 ng/mm³, about 10 ng/mm³, about 11 ng/mm³, about 12 ng/mm³, about 13 ng/mm³, about 14 ng/mm³, about 15 ng/mm³, about 16 ng/mm³, about 17 ng/mm³, about 18 ng/mm³, about 19 ng/mm³, about 20 ng/mm³, about 25 ng/mm³, about 30 ng/mm³, about 35 ng/mm³, about 40 ng/mm³, about 45 ng/mm³, about 50 ng/mm³, about 55 ng/mm³, about 60 ng/mm³, about 65 ng/mm³, about 70 ng/mm³, about 75 ng/mm³, about 80 ng/mm³, about 85 ng/mm³, about 90 ng/mm³, about 95 ng/mm³, about 100 ng/mm³, about 105 ng/mm³, about 110 ng/mm³, about 115 ng/mm³, about 120 ng/mm³, about 125 ng/mm³, or more. As another example, a fluid medium adsorbed into a scaffold can contain LPS a concentration of about 12.5 ng/mm³. It is understood that recitation of the above discrete values includes a range between each recited value.

In some embodiments, a fluid medium containing LPS is adsorbed into a scaffold. A fluid medium adsorbed into a scaffold can contain LPS at a concentration of about 10 ng/ml to about 1,000 ng/ml. For example, a fluid medium adsorbed into a scaffold can contain LPS at a concentration of about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, about 150 ng/ml, about 160 ng/ml, about 170 ng/ml, about 180 ng/ml, about 190 ng/ml, about 200 ng/ml, about 250 ng/ml, about 300 ng/ml, about 350 ng/ml, about 400 ng/ml, about 450 ng/ml, about 500 ng/ml, about 550 ng/ml, about 600 ng/ml, about 650 ng/ml, about 700 ng/ml, about 750 ng/ml, about 800 ng/ml, about 850 ng/ml, about 900 ng/ml, about 950 ng/ml, about 1,000 ng/ml, or more. As another example, a fluid medium adsorbed into a scaffold can contain LPS a concentration of about 100 ng/ml. It is understood that recitation of the above discrete values includes a range between each recited value.

Tumor Necrosis Factor Alpha.

Tumor necrosis factor alpha (TNFα, also known as simply “TNF” given that TNFβ is now known as lymphotoxin, LT) can promote the M1 phenotype of macrophages. TNFα is a type II transmembrane protein involved in systemic inflammation and can be produced by activated M1 macrophages, CD4+ lymphocytes, NK cells and neurons. Soluble homotrimeric cytokine (sTNF) can be released via proteolytic cleavage (for the purposes of the present disclosure, “TNFα” can include soluble forms of TNF). The sequence and structure of TNFα is well characterized in the art. TNFα can bind two receptors, TNFR1 and TNFR2, resulting in conformational changes and dissociation enabling TRADD adapter protein (TNFR-Associated Death Domain) binding and stimulation of subsequent related signal cascades for cell survival, apoptosis, inflammatory responses, and cellular differentiation. Soluble TNFα (e.g., secreted TNF or sTNF) is commercially available (see e.g., Enzo Life Sciences, Farmingdale, N.Y.). TNFα can be formulated as generally described herein.

In some embodiments, TNFα can be attached (e.g., covalently bound) in or on a scaffold. TNFα can be present in or on a scaffold at a concentration of about 0.1 ng/mm³ to about 125 ng/mm³. For example, TNFα can be present in or on a scaffold at a concentration of about 0.1 ng/mm³, about 1 ng/mm³, about 2 ng/mm³, about 3 ng/mm³, about 4 ng/mm³, about 5 ng/mm³, about 6 ng/mm³, about 7 ng/mm³, about 8 ng/mm³, about 9 ng/mm³, about 10 ng/mm³, about 11 ng/mm³, about 12 ng/mm³, about 13 ng/mm³, about 14 ng/mm³, about 15 ng/mm³, about 16 ng/mm³, about 17 ng/mm³, about 18 ng/mm³, about 19 ng/mm³, about 20 ng/mm³, about 25 ng/mm³, about 30 ng/mm³, about 35 ng/mm³, about 40 ng/mm³, about 45 ng/mm³, about 50 ng/mm³, about 55 ng/mm³, about 60 ng/mm³, about 65 ng/mm³, about 70 ng/mm³, about 75 ng/mm³, about 80 ng/mm³, about 85 ng/mm³, about 90 ng/mm³, about 95 ng/mm³, about 100 ng/mm³, about 105 ng/mm³, about 110 ng/mm³, about 115 ng/mm³, about 120 ng/mm³, about 125 ng/mm³, or more. As another example, a fluid medium adsorbed into a scaffold can contain TNFα a concentration of about 12.5 ng/mm³. It is understood that recitation of the above discrete values includes a range between each recited value.

In some embodiments, a fluid medium containing TNFα is adsorbed into a scaffold. A fluid medium adsorbed into a scaffold can contain TNFα at a concentration of about 10 ng/ml to about 1,000 ng/ml. For example, a fluid medium adsorbed into a scaffold can contain TNFα at a concentration of about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, about 150 ng/ml, about 160 ng/ml, about 170 ng/ml, about 180 ng/ml, about 190 ng/ml, about 200 ng/ml, about 250 ng/ml, about 300 ng/ml, about 350 ng/ml, about 400 ng/ml, about 450 ng/ml, about 500 ng/ml, about 550 ng/ml, about 600 ng/ml, about 650 ng/ml, about 700 ng/ml, about 750 ng/ml, about 800 ng/ml, about 850 ng/ml, about 900 ng/ml, about 950 ng/ml, about 1,000 ng/ml, or more. As another example, a fluid medium adsorbed into a scaffold can contain TNFα at a concentration of about 100 ng/ml. It is understood that recitation of the above discrete values includes a range between each recited value.

Interleukin-4.

Interleukin-4 (IL4) can promote the M2A phenotype of macrophages. IL4 is an interleukin cytokine that binds the Interleukin-4 receptor. IL4 can also inhibit classical activation of macrophages into an M1 phenotype. The sequence and structure of IL4 is well characterized in the art. IL4 is commercially available (see e.g., Prospec, East Brunswick, N.J.; Creative BioMart, Shirley, N.Y.). IL4 can be formulated as generally described herein.

In some embodiments, IL4 can be attached (e.g., covalently bound) in or on a scaffold. IL4 can be present in or on a scaffold at a concentration of about 0.1 ng/mm³ to about 125 ng/mm³. For example, IL4 can be present in or on a scaffold at a concentration of about 0.1 ng/mm³, about 1 ng/mm³, about 2 ng/mm³, about 3 ng/mm³, about 4 ng/mm³, about 5 ng/mm³, about 6 ng/mm³, about 7 ng/mm³, about 8 ng/mm³, about 9 ng/mm³, about 10 ng/mm³, about 11 ng/mm³, about 12 ng/mm³, about 13 ng/mm³, about 14 ng/mm³, about 15 ng/mm³, about 16 ng/mm³, about 17 ng/mm³, about 18 ng/mm³, about 19 ng/mm³, about 20 ng/mm³, about 25 ng/mm³, about 30 ng/mm³, about 35 ng/mm³, about 40 ng/mm³, about 45 ng/mm³, about 50 ng/mm³, about 55 ng/mm³, about 60 ng/mm³, about 65 ng/mm³, about 70 ng/mm³, about 75 ng/mm³, about 80 ng/mm³, about 85 ng/mm³, about 90 ng/mm³, about 95 ng/mm³, about 100 ng/mm³, about 105 ng/mm³, about 110 ng/mm³, about 115 ng/mm³, about 120 ng/mm³, about 125 ng/mm³, or more. As another example, a fluid medium adsorbed into a scaffold can contain IL4 a concentration of about 12.5 ng/mm³. It is understood that recitation of the above discrete values includes a range between each recited value.

In some embodiments, a fluid medium containing IL4 is adsorbed into a scaffold. A fluid medium adsorbed into a scaffold can contain IL4 at a concentration of about 1 ng/ml to about 400 ng/ml. For example, a fluid medium adsorbed into a scaffold can contain IL4 at a concentration of about 1 ng/ml, about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 150 ng/ml, about 200 ng/ml, about 250 ng/ml, about 300 ng/ml, about 350 ng/ml, about 400 ng/ml, or more. As another example, a fluid medium adsorbed into a scaffold can contain IL4 at a concentration of about 40 ng/ml. It is understood that recitation of the above discrete values includes a range between each recited value.

Interleukin-13.

Interleukin-13 (IL13) can promote the M2A phenotype of macrophages. IL13 has similar effects as IL4. The sequence and structure of IL13 is well characterized in the art. IL13 is commercially available (see e.g., Prospec, East Brunswick, N.J.; Creative BioMart, Shirley, N.Y.). IL13 can be formulated as generally described herein.

In some embodiments, IL13 can be attached (e.g., covalently bound) in or on a scaffold. IL13 can be present in or on a scaffold at a concentration of about 0.1 ng/mm³ to about 125 ng/mm³. For example, IL13 can be present in or on a scaffold at a concentration of about 0.1 ng/mm³, about 1 ng/mm³, about 2 ng/mm³, about 3 ng/mm³, about 4 ng/mm³, about 5 ng/mm³, about 6 ng/mm³, about 7 ng/mm³, about 8 ng/mm³, about 9 ng/mm³, about 10 ng/mm³, about 11 ng/mm³, about 12 ng/mm³, about 13 ng/mm³, about 14 ng/mm³, about 15 ng/mm³, about 16 ng/mm³, about 17 ng/mm³, about 18 ng/mm³, about 19 ng/mm³, about 20 ng/mm³, about 25 ng/mm³, about 30 ng/mm³, about 35 ng/mm³, about 40 ng/mm³, about 45 ng/mm³, about 50 ng/mm³, about 55 ng/mm³, about 60 ng/mm³, about 65 ng/mm³, about 70 ng/mm³, about 75 ng/mm³, about 80 ng/mm³, about 85 ng/mm³, about 90 ng/mm³, about 95 ng/mm³, about 100 ng/mm³, about 105 ng/mm³, about 110 ng/mm³, about 115 ng/mm³, about 120 ng/mm³, about 125 ng/mm³, or more. As another example, a fluid medium adsorbed into a scaffold can contain IL13 a concentration of about 12.5 ng/mm³. It is understood that recitation of the above discrete values includes a range between each recited value.

In some embodiments, a fluid medium containing IL13 is adsorbed into a scaffold. A fluid medium adsorbed into a scaffold can contain IL13 at a concentration of about 1 ng/ml to about 400 ng/ml. For example, a fluid medium adsorbed into a scaffold can contain IL13 at a concentration of about 1 ng/ml, about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 150 ng/ml, about 200 ng/ml, about 250 ng/ml, about 300 ng/ml, about 350 ng/ml, about 400 ng/ml, or more. As another example, a fluid medium adsorbed into a scaffold can contain IL13 at a concentration of about 20 ng/ml. It is understood that recitation of the above discrete values includes a range between each recited value.

Interleukin-10.

Interleukin-10 (IL10) can promote the M2C phenotype of macrophages. IL10, also known as human cytokine synthesis inhibitory factor (CSIF), is an anti-inflammatory cytokine with pleiotropic effects in immunoregulation and inflammation. IL-10 can bind Interleukin 10 receptor, alpha subunit. IL-10 can inhibit synthesis of pro-inflammatory cytokines such as IFN-γ, IL-2, IL-3, TNFα and GM-CSF. IL10 is encoded by the IL10 gene. The sequence and structure of IL10 is well characterized in the art. IL10 is commercially available (see e.g., Prospec, East Brunswick, N.J.; Novoprotein, Shanghai, China). IL10 can be formulated as generally described herein.

In some embodiments, IL10 can be attached (e.g., covalently bound) in or on a scaffold. IL10 can be present in or on a scaffold at a concentration of about 0.1 ng/mm³ to about 125 ng/mm³. For example, IL10 can be present in or on a scaffold at a concentration of about 0.1 ng/mm³, about 1 ng/mm³, about 2 ng/mm³, about 3 ng/mm³, about 4 ng/mm³, about 5 ng/mm³, about 6 ng/mm³, about 7 ng/mm³, about 8 ng/mm³, about 9 ng/mm³, about 10 ng/mm³, about 11 ng/mm³, about 12 ng/mm³, about 13 ng/mm³, about 14 ng/mm³, about 15 ng/mm³, about 16 ng/mm³, about 17 ng/mm³, about 18 ng/mm³, about 19 ng/mm³, about 20 ng/mm³, about 25 ng/mm³, about 30 ng/mm³, about 35 ng/mm³, about 40 ng/mm³, about 45 ng/mm³, about 50 ng/mm³, about 55 ng/mm³, about 60 ng/mm³, about 65 ng/mm³, about 70 ng/mm³, about 75 ng/mm³, about 80 ng/mm³, about 85 ng/mm³, about 90 ng/mm³, about 95 ng/mm³, about 100 ng/mm³, about 105 ng/mm³, about 110 ng/mm³, about 115 ng/mm³, about 120 ng/mm³, about 125 ng/mm³, or more. As another example, a fluid medium adsorbed into a scaffold can contain IL10 a concentration of about 12.5 ng/mm³. It is understood that recitation of the above discrete values includes a range between each recited value.

In some embodiments, a fluid medium containing IL10 is adsorbed into a scaffold. A fluid medium adsorbed into a scaffold can contain IL10 at a concentration of about 1 ng/ml to about 400 ng/ml. For example, a fluid medium adsorbed into a scaffold can contain IL10 at a concentration of about 1 ng/ml, about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 150 ng/ml, about 200 ng/ml, about 250 ng/ml, about 300 ng/ml, about 350 ng/ml, about 400 ng/ml, or more. As another example, a fluid medium adsorbed into a scaffold can contain IL10 at a concentration of about 40 ng/ml. It is understood that recitation of the above discrete values includes a range between each recited value.

Coupling

As described herein, one or more biomolecules can be coupled, conjugated or bound to a matrix material of a scaffold. Coupling, conjugation, or binding of a biomolecule and a matrix material are well known in the art. Except as otherwise noted herein, therefore, the subject matter of the present disclosure can be carried out in accordance with such known processes.

In some embodiments, one or more biomolecules (e.g., IFNy, LPS, TNFα, IL4, or IL10) can be chemically bound to a matrix material of a scaffold. A chemical bond is understood as an attraction between atoms of a biomolecule and atoms of a matrix material that allows the formation of a linkage between atoms of the biomolecule and the matrix material. A bond can be caused by an electrostatic force of attraction between opposite charges, either between electrons and nuclei, or as the result of a dipole attraction. A bond (e.g., between a biomolecule and a matrix material) can be, for example, a covalent bond, a coordinate covalent bond, an ionic bond, polar covalent, a dipole-dipole interaction, a London dispersion force, a cation-pi interaction, or hydrogen bonding.

Biomolecules described herein can be incorporated onto or into the matrix material of a scaffold, causing the biomolecules to be attached on or embedded within. Chemical modification methods can be used to link (e.g., covalently link) a biomolecule on the surface or interior of a matrix material of a scaffold. Surface functional groups of a matrix component can be coupled with reactive functional groups of a biomolecule to form covalent bonds using coupling agents well known in the art such as aldehyde compounds, carbodiimides, and the like. Additionally, a spacer molecule can be used to gap surface reactive groups (e.g., in collagen) and the reactive groups of the biomolecules to allow more flexibility of such molecules, e.g., on the surface of the matrix. Other similar methods of attaching biomolecules to the interior or exterior of a matrix will be known to one of skill in the art.

In some embodiments, one or more biomolecules (e.g., IFNy, LPS, TNFα, IL4, or IL10) can be conjugated to a matrix material of a scaffold. For example, one or more biomolecules (e.g., IFNy, LPS, TNFα, IL4, or IL10) can be degradably conjugated to a scaffold, which would allow their release from the scaffolds. For example, a biomolecule can be biodegradably conjugated to matrix material. As another example, a biomolecule can be biodegradably conjugated to matrix material via an ester, amide, or ether bond. A biodegradably conjugated biomolecule-matrix material can be non-toxic, capable of maintaining sufficient mechanical integrity until degraded, or capable of controlled rates of degradation. Factors that can influence degradation rate include percent crystallinity, molecular weight, or hydrophobicity. Degradation rate can depend on location in the body, which influences the environment surrounding the polymer such as pH, enzymes concentration, and amount of water among others.

One or more biomolecules can be attached to a scaffold via biotin-streptavidin or -avidin interaction (see e.g., Example 9).

A streptavidin can be a protein having a high affinity for biotin (e.g., Kd of about 10⁻¹⁴ mol/L). A streptavidin or a nucleotide encoding such, can be isolated from the bacterium Streptomyces (e.g., Streptomyces avidinii). A streptavidin can be any commercially available streptavidin (e.g., Invitrogen; Qiagen; Thermo Scientific; Jackson ImmunoResearch; Sigma Aldrich; Cell Signaling Technology). A streptavidin can be a variant of a naturally occurring streptavidin having at least about 80%, 85%, 90%, 95%, or 99% sequence identity thereto and retaining or substantially retaining high affinity for biotin. A streptavidin can be a tetramer, with each subunit binding a biotin with equal or substantially equal affinity. A streptavidin can have a mildly acidic isoelectric point (pI) (e.g., about 5). A streptavidin can lack any carbohydrate modification. Where a streptavidin has no carbohydrate modification and a near-neutral pI, it can have substantially lower nonspecific binding compared to avidin.

A streptavidin can be a streptavidin variant. For example, a streptavidin can be a monovalent, divalent, and trivalent variant. As another example, a variant streptavidin can have a near-neutral pI.

An avidin can be a protein having a high affinity for biotin (e.g., Kd of about 10⁻¹⁵ mol/L). An avidin or a nucleotide encoding such, can be isolated from egg white. Wild type avidin has about 30% sequence identity to wild type streptavidin, but highly similar secondary, tertiary and quaternary structure. An avidin can be glycosylated, positively charged, or have pseudo-catalytic activity (i.e., enhance alkaline hydrolysis of an ester linkage between biotin and a nitrophenyl group) or can have a higher tendency for aggregation as compared to a streptavidin. An avidin can be a tetramer of about 66-69 kDa in size. An avidin can have about 10% of molecular weight attributed to carbohydrate content composed of about 4 to 5 mannose or about three N-acetylglucosamine residues.

An avidin can be a streptavidin variant. For example, an avidin can be a non-glycosylated avidin. As another example, an avidin can be a deglycosylated avidin (e.g., Neutravidin), which can be more comparable to the size, pI or nonspecific binding of a wild type streptavidin. As another example, an avidin can be a deglycosylated avidin having modified arginines, exhibiting a more neutral isoelectric point (pI) and can better overcome problems of non-specific binding. Deglycosylated, neutral forms of avidin are commercially available (e.g., Extravidin, Sigma-Aldrich; Neutravidin, Thermo Scientific or Invitrogen; NeutraLite, Belovo). An avidin can have reversible binding characteristics through nitration or iodination of a binding site tyrosine, or exhibit strong biotin binding characteristics at about pH 4 or biotin release at a pH of about 10 or higher. An avidin can be a monovalent, divalent, and trivalent variant of avidin.

A biotin can be a water soluble B-complex vitamin (e.g., vitamin B7, vitamin H, or coenzyme R). A biotin can be a heterocyclic sulfur-containing (mono-)carboxylic acid. A biotin can comprise an imidazole ring and thiophene ring fused. A biotin can comprise a ureido (tetrahydroimidizalone) ring fused with a tetrahydrothiophene ring, optionally with a veleric acid substituent on a carbon of the tetrahydrothiophene ring. Streptavidin or avidin can bind biotin with high affinity (e.g., Kd of 10⁻¹⁴ mol/l to 10⁻¹⁵ mol/l) and specificity.

A biotin can be any commercially available biotin (e.g., Invitrogen; Qiagen; Thwermo Scientific; Jackson ImmunoResearch; Sigma Aldrich; Cell Signalling Technology). A biotin can be a variant compound of a naturally occurring biotin that retains or substantially retaining high affinity for streptavidin.

A biotin can have a structural formula according to C10H16O3N2S. A biotin can have a structure as follows:

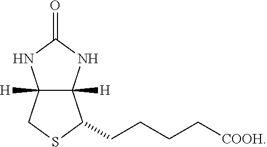 [SMILES: O=C(O)CCCC[C@@H]1SC[C@@H]2NC(=O)N[C@@H]21]

Biotin can be attached to a molecule or substrate by biotinylation. Biotinylated proteins of interest can be isolated from a sample by exploiting this highly stable interaction.

Biotinylation is the process of covalently attaching a biotin to a molecule or substrate. Biotinylation is generally rapid, specific and is unlikely to perturb the natural function of the molecule or substrate to which it is attached given the small size of a biotin (e.g., MW=244.31 g/mol). Biotin can bind to streptavidin or avidin with an extremely high affinity, fast on-rate, and high specificity, and these interactions can be exploited as described herein. Biotin-binding to streptavidin or avidin can be resistant to extremes of heat, pH, or proteolysis, which can allow use of a biotinylated molecule or substrate in a wide variety of environments. Furthermore, multiple biotin molecules can be conjugated to a molecule or substrate, which can allow binding of multiple streptavidin, avidin, or Neutravidin. A large number of biotinylation reagents are know in the art and commercially available.

Various assays are available to determine extent of biotinylation.

The HABA (2-(4-hydroxyazobenzene) benzoic acid) assay can be used to determine the extent of biotinylation. HABA dye is bound to avidin or streptavidin and yields a characteristic absorbance. When biotinylated proteins or other molecules are introduced, the biotin displaces the dye, resulting in a change in absorbance at 500 nm. This change is directly proportional to the level of biotin in the sample. A HABA assay can require a relatively large amount of sample.

Extent of biotinylation can also be measured by streptavidin gel-shift, since streptavidin remains bound to biotin during agarose gel electrophoresis or polyacrylamide gel electrophoresis. The proportion of target biotinylated can be measured via the change in band intensity of the target with or without excess streptavidin, seen quickly and quantitatively by Coomassie Brilliant Blue staining.

Biotinylation, also called biotin labeling, is most commonly performed through chemical means, although enzymatic methods are also available. Chemical biotinylation can use various conjugation chemistries to yield a nonspecific biotinylation of amines, carboxylates, sulfhydryls or carbohydrates (e.g., NHS-coupling gives biotinylation of a primary amines). Chemical biotinylation reagents can include a reactive group attached via a linker to the valeric acid side chain of biotin. Because the biotin binding pocket in avidin or streptavidin is buried beneath the protein surface, a biotinylation reagent possessing a longer linker can be desirable, as such longer linker can enable the biotin molecule to be more accessible to binding avidin, streptavidin, or Neutravidin. A linker can also mediate the solubility of a biotinylation reagent. Linkers that incorporate poly(ethylene) glycol (PEG) can make water-insoluble reagents soluble or increase the solubility of biotinylation reagents that are already soluble to some extent.

Primary Amine Biotinylation.

Biotin can be conjugated to an amine group on the molecule or substrate. A primary amine group can be present as a lysine side chain epsilon-amine or N-terminal α-amine. Amine-reactive biotinylation reagents can be divided into two groups based on water solubility.

N-hydroxysuccinimide (NHS) esters have poor solubility in aqueous solutions. For reactions in aqueous solution, NHS can be first be dissolved in an organic solvent, then diluted into the aqueous reaction mixture. Commonly used organic solvents for this purpose can include dimethyl sulfoxide (DMSO) and dimethyl formamide (DMF). Because of the hydrophobicity of NHS-esters, NHS biotinylation reagents can also diffuse through the cell membrane, meaning that they will biotinylate both internal and external components of a cell.

Sulfo-NHS esters are more soluble in water and can be dissolved in water just before use because they hydrolyze easily. The water solubility of sulfo-NHS-esters is due at least in part from a sulfonate group on the N-hydroxysuccinimide ring. Water solubility can eliminate a need to dissolve the reagent in an organic solvent. Sulfo-NHS-esters of biotin do not penetrate the cell membrane.

The chemical reactions of NHS- and sulfo-NHS esters can be identical, in that they can both react spontaneously with amines to form an amide bond. Because the target for the ester is a deprotonated primary amine, the reaction is favored under basic conditions (above pH 7). Hydrolysis of the NHS ester is a major competing reaction, and the rate of hydrolysis increases with increasing pH. NHS- and sulfo-NHS-esters have a half-life of several hours at pH 7 but only a few minutes at pH 9.

There is additional flexibility in the conditions for conjugating NHS-esters to primary amines. Incubation temperatures can range from about 4-37° C., pH values in the reaction range from about 7-9, or incubation times range from a few minutes to about 12 hours. Buffers containing amines (e.g., Tris or glycine) can be avoided, because they compete with the reaction.

Sulfhydryl Biotinylation

An alternative to primary amine biotinylation is to label sulfhydryl groups with biotin. Sulfhydryl-reactive groups such as maleimides, haloacetyls, or pyridyl disulfides, can require free sulfhydryl groups for conjugation; disulfide bonds can be first reduced to free up the sulfhydryl groups for biotinylation. If no free sulfhydryl groups are available, lysines can be modified with various thiolation reagents (Traut's Reagent, SAT(PEG4), SATA and SATP), resulting in the addition of a free sulfhydryl. Sulfhydryl biotinylation can be performed at a slightly lower pH (e.g., about 6.5-7.5) than labeling with NHS esters.

Carboxyl Biotinylation.

Biotinylation reagents that target carboxyl groups do not have a carboxyl-reactive moiety per se but instead rely on a carbodiimide crosslinker such as EDC to bind the primary amine on a biotinylation reagent to a carboxyl group on the target.

Biotinylation at carboxyl groups can occur at a pH of about 4.5-5.5. To prevent crossreactivity of the crosslinker with buffer constituents, buffers should not contain primary amines (e.g., Tris, glycine) or carboxyls (e.g., acetate, citrate).

Glycoprotein Biotinylation

Glycoproteins can be biotinylated by modifying the carbohydrate residues to aldehydes, which can then react with hydrazine- or alkoxyamine-based biotinylation reagents. Sodium periodate can oxidize a sialic acid on glycoproteins to aldehydes to form these stable linkages at a pH of about 4-6.

Antibodies can be heavily glycosylated, and because glycosylation does not interfere with the antibody activity, biotinylating the glycosyl groups can be an ideal strategy to generate biotinylated antibodies.

Biotinylation at carboxyl groups can occur at a pH of about 4.5-5.5. To prevent crossreactivity of the crosslinker with buffer constituents, buffers should not contain primary amines (e.g., Tris, glycine) or carboxyls (e.g., acetate, citrate).

Oligonucleotide Biotinylation.

Oligonucleotides can be readily biotinylated in the course of oligonucleotide synthesis by the phosphoramidite method using, e.g., commercial biotin phosphoramidite. Upon the standard deprotection, the conjugates obtained can be purified using reverse-phase or anion-exchange HPLC.

Non-Specific Biotinylation.

Photoactivatable biotinylation reagents can be useful when primary amines, sulfhydryls, carboxyls or carbohydrates are not available or not desired for labeling. A photoactivatable biotinylation reagent relies on aryl azides, which become activated by ultraviolet light (UV; >350 nm), which then react at C—H and N—H bonds. A photoactivatable biotinylation reagent can also be used to activate biotinylation at specific times by simply exposing the reaction to UV light at the specific time or condition.

Processes for coupling, conjugating, or binding a receptor or ligand, such as avidin or streptavidin, to a matrix material, scaffold, or biomolecule are well known (see e.g. Savage 1992, Avidin-Biotin Chemistry: A Handbook, Pierce Chemical Co, ISBN-10 0935940111, ISBN-13 978-0935940114; McMahon 2010 Avidin-Biotin Interactions: Methods and Applications, Humana Press, ASIN B00GA4420E; Hermanson 2010 Bioconjugate Techniques, Academic Press, ASIN B005YXETUU). Except as otherwise noted herein, therefore, the process of the present disclosure can be carried out in accordance with such processes.

Controlled Release

In some embodiments, compositions for promoting M1 macrophage or M2 macrophage phenotypes are control released. As described herein, sequential promotion of an M1 macrophage phenotype followed by promotion of an M2 macrophage phenotype can increase vascularization of a scaffold. For example, a controlled release composition for promoting M1 macrophage or M2 macrophage phenotypes can be introduced into or onto a scaffold. As another example, a controlled release composition for promoting an M1 macrophage phenotype can be introduced into or onto a scaffold. As another example, a controlled release composition for promoting an M2 macrophage phenotype can be introduced into or onto a scaffold.

In some embodiments, one or more biomolecules (e.g., IFNy, LPS, TNFα, IL4, or IL10) can be conjugated to a scaffold (e.g., covalently or through a linkage). For example, one or more biomolecules (e.g., IFNy, LPS, TNFα, IL4, or IL10) can be degradably conjugated to a scaffold, which would allow their release from the scaffolds. For example, one or more biomolecules can be attached to a scaffold via biotin-streptavidin interaction (see e.g., Example 9). Biotinylation can be a useful strategy in bioconjugation techniques because the small size of biotin can limit damage to protein bioactivity.

The release of drug from affinity-based systems can be described by

$$\frac{\partial C}{\partial t} = - \frac{D}{{K}_{b} + 1} {∇}^{2} C$$

where C is the concentration of the free drug, D is its diffusivity, and Kb is the ratio of the concentration of available binding sites to the dissociation constant Kd. The assumptions of this model are that the drug diffuses with constant diffusivity, that the drug binds to its receptor with a 1:1 interaction, which is the case with a high ratio of available binding sites to the diffusible drug, and that the rate of binding is much higher than that of dissociation. Because of the extremely low Kd of biotin-streptavidin (10⁻¹⁵ M), with a D˜10-9 m²/s for IL4, and with 0.04 mol/m³ of avidin added to the system, this model predicts that biotinylated drug would virtually never exit the system, a result of strong binding interactions between biotin and streptavidin.

Surprisingly, results described herein show that biotinylated IL4 was slowly released over 6 days, and there was no evidence of remaining IL4 after 2 weeks in vivo. These results support that the conjugation of IL4 to biotin substantially reduced its binding affinity to streptavidin, which is in agreement with studies on the use of streptavidin-biotin interactions in affinity separation chromatography.

As other examples, affinity systems with weaker binding interactions, one or more biomolecules can be attached to a scaffold via heparin with heparin-binding growth factors, cyclodextrins with small hydrophobic drugs, or antibody-antigen pairs. Such controlled release strategies are known in the art and can be modified accordingly.

As described herein, rapid release of IFN-gamma caused early M1 polarization of macrophages in vitro, at least in terms of gene expression, and sustained release of IL4 caused M2 polarization that persisted at 6 days in terms of both gene expression and protein secretion. But as described herein, early release of IFN-gamma combined with sustained release of IL4 (Combo group) did not result in robust M1 and M2 polarization at early and late time points, respectively, and protein secretion at any time point was not different from the negative control. While under no obligation to provide a mechanism, and in no way limiting the scope of the present disclosure, it is thought that the effects of IFNg and IL4 were competing. M2 macrophages are present at early time points and mixed macrophage phenotypes have been shown to be beneficial for angiogenesis, but the two phases may need to be more temporally separated in future generations of these biomaterials in order to allow more robust sequential polarization.

A controlled release systems described herein can allow for controlled release of separate chemicals or compositions at similar or at different rates. For example, a controlled release system can allow the release of separate chemicals or compositions at different rates, so as to provide, e.g., an initial higher concentration of a composition promoting an M1 macrophage phenotype followed by a later higher concentration of a composition promoting an M2 macrophage phenotype. As another example, a controlled release system as described herein can provide for the delivery of one compound or composition sooner than a second compound or composition. As a specific example, a controlled release system described herein can release a portion or a substantial portion of a composition promoting an M1 macrophage phenotype earlier than a composition promoting an M2 macrophage phenotype. For example, a composition promoting an M1 macrophage phenotype can be released about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, or about 10 days or more days before a composition promoting an M2 macrophage phenotype.

Compositions described herein (e.g., a composition promoting an M1 macrophage phenotype; or a composition promoting an M2 macrophage phenotype) can be introduced into or onto a scaffold via a carrier based system, such as an encapsulation vehicle. For example, a composition can be encapsulated within a polymeric delivery systems so as to provide for controlled release of such compositions from within the scaffold. Such vehicles are useful as slow release compositions. For example, various compositions can be micro-encapsulated to provide for enhanced stability or prolonged delivery. Encapsulation vehicles include, but are not limited to, microparticles, liposomes, microspheres, or the like, or a combination of any of the above to provide the desired release profile in varying proportions. Other methods of controlled-release delivery of agents will be known to the skilled artisan. Moreover, these and other systems can be combined or modified to optimize the integration/release of agents within the scaffold.

For example, the polymeric delivery system can be a polymeric microsphere, preferably a PLGA polymeric microspheres. A variety of polymeric delivery systems, as well as methods for encapsulating a molecule such as a growth factor, are known to the art (see e.g., Varde and Pack (2004) Expert Opin Biol Ther 4, 35-51). Polymeric microspheres can be produced using naturally occurring or synthetic polymers and are particulate systems in the size range of 0.1 to 500 μm. Polymeric micelles and polymeromes are polymeric delivery vehicles with similar characteristics to microspheres and can also facilitate encapsulation and matrix integration of the compounds described herein. Fabrication, encapsulation, and stabilization of microspheres for a variety of payloads are within the skill of the art (see e.g., Varde & Pack (2004) Expert Opin. Biol. 4(1) 35-51). The release rate of the microspheres can be tailored by type of polymer, polymer molecular weight, copolymer composition, excipients added to the microsphere formulation, and microsphere size. Polymer materials useful for forming microspheres include PLA, PLGA, PLGA coated with DPPC, DPPC, DSPC, EVAc, gelatin, albumin, chitosan, dextran, DL-PLG, SDLMs, PEG (e.g., ProMaxx), sodium hyaluronate, diketopiperazine derivatives (e.g., Technosphere), calcium phosphate-PEG particles, and/or oligosaccharide derivative DPPG (e.g., Solidose). Encapsulation can be accomplished, for example, using a water/oil single emulsion method, a water-oil-water double emulsion method, or lyophilization. Several commercial encapsulation technologies are available (e.g., ProLease®, Alkerme).

Liposomes can also be used to integrate compositions described herein with a scaffold. The agent carrying capacity and release rate of liposomes can depend on the lipid composition, size, charge, drug/lipid ratio, and method of delivery. Conventional liposomes are composed of neutral or anionic lipids (natural or synthetic). Commonly used lipids are lecithins such as phosphatidylcholines, phosphatidylethanolamines, sphingomyelins, phosphatidylserines, phosphatidylglycerols, and phosphatidylinositols. Liposome encapsulation methods are commonly known in the arts (Galovic et al. (2002) Eur. J. Pharm. Sci. 15, 441-448; Wagner et al. (2002) J. Liposome Res. 12, 259-270). Targeted liposomes and reactive liposomes can also be used in combination with the agents and matrix. Targeted liposomes have targeting ligands, such as monoclonal antibodies or lectins, attached to their surface, allowing interaction with specific receptors and/or cell types. Reactive or polymorphic liposomes include a wide range of liposomes, the common property of which is their tendency to change their phase and structure upon a particular interaction (e.g., pH-sensitive liposomes) (see e.g., Lasic (1997) Liposomes in Gene Delivery, CRC Press, FL).

Cell Seeding

In some embodiments, a scaffold can be seeded with one or more types of cells, such as a progenitor cell. Progenitor cells can be introduced (e.g., implanted, injected, infused, or seeded) into or onto an artificial structure (e.g., a scaffold comprising a matrix material) capable of supporting three-dimensional tissue or organ formation. Different types of cells (e.g., progenitor cells) can be co-introduced or sequentially introduced. Different types of cells can be introduced in the same spatial position, similar spatial positions, or different spatial positions, relative to each other. For example, different types of cells can be introduced into or onto different areas of the matrix material. It is contemplated that more than one type of cell can be introduced into the matrix.

Cells can be introduced into the matrix material by a variety of means known to the art. Methods for the introduction (e.g., infusion, seeding, injection, etc.) of cells into or into the matrix material are discussed in, for example, Ma and Elisseeff, ed. (2005) Scaffolding In Tissue Engineering, CRC, ISBN 1574445219; Saltzman (2004) Tissue Engineering: Engineering Principles for the Design of Replacement Organs and Tissues, Oxford ISBN 019514130X; Minuth et al. (2005) Tissue Engineering: From Cell Biology to Artificial Organs, John Wiley & Sons, ISBN 3527311866. For example, cells can be introduced into or onto the matrix by methods including hydrating freeze-dried scaffolds with a cell suspension (e.g., at a concentration of 100 cells/ml to several million cells/ml).

Methods of culturing and differentiating progenitor cells in or on scaffolds are generally known in the art (see e.g., Saltzman (2004) Tissue Engineering: Engineering Principles for the Design of Replacement Organs and Tissues, Oxford ISBN 019514130X; Vunjak-Novakovic and Freshney, eds. (2006) Culture of Cells for Tissue Engineering, Wiley-Liss, ISBN 0471629359; Minuth et al. (2005) Tissue Engineering: From Cell Biology to Artificial Organs, John Wiley & Sons, ISBN 3527311866). As will be appreciated by one skilled in the art, the time between cell introduction into or onto the matrix and engrafting the resulting matrix can vary according to particular application. Incubation (and subsequent replication and/or differentiation) of the engineered composition containing cells in or on the matrix material can be, for example, at least in part in vitro, substantially in vitro, at least in part in vivo, or substantially in vivo. Determination of optimal culture time is within the skill of the art. A suitable medium can be used for in vitro cell infusion, differentiation, or cell trans-differentiation (see e.g., Vunjak-Novakovic and Freshney, eds. (2006) Culture of Cells for Tissue Engineering, Wiley-Liss, ISBN 0471629359; Minuth et al. (2005) Tissue Engineering: From Cell Biology to Artificial Organs, John Wiley & Sons, ISBN 3527311866). The culture time can vary from about an hour, several hours, a day, several days, a week, or several weeks. The quantity and type of cells present in the matrix can be characterized by, for example, morphology by ELISA, by protein assays, by genetic assays, by mechanical analysis, by RT-PCR, and/or by immunostaining to screen for cell-type-specific markers (see e.g., Minuth et al. (2005) Tissue Engineering: From Cell Biology to Artificial Organs, John Wiley & Sons, ISBN 3527311866).

Progenitor Cells

Compositions and methods described herein can employ progenitor cells. Such cells can be isolated, purified, or cultured by a variety of means known to the art (see e.g., Vunjak-Novakovic and Freshney (2006) Culture of Cells for Tissue Engineering, Wiley-Liss, ISBN 0471629359). In some aspects, progenitor cells can be derived from the same or different species as an intended transplant recipient. For example, progenitor cells can be derived from an animal, including, but not limited to, a vertebrate such as a mammal, a reptile, or an avian. In some configurations, a mammal or avian is preferably a horse, a cow, a dog, a cat, a sheep, a pig, or a chicken, and most preferably a human.

Progenitor cells of the present teachings include cells capable of differentiating into a target tissue or organ, or undergoing morphogenesis to form a target tissue or organ. Non-limiting examples of tissue progenitor cells include mesenchymal stem cells (MSCs), cells differentiated from MSCs, osteoblasts, chondrocytes, myocytes, adipocytes, neuronal cells, neuronal supporting cells such as neural glial cells (such as Schwann cells), fibroblastic cells such as interstitial fibroblasts, tendon fibroblasts, dermal fibroblasts, ligament fibroblasts, periodontal fibroblasts such as gingival fibroblasts, craniofacial fibroblasts, cardiomyocytes, epithelial cells, liver cells, uretheral cells, kidney cells, periosteal cells, bladder cells, beta-pancreatic islet cell, odontoblasts, dental pulp cells, periodontal cells, lung cells, or cardiac cells. Vascular progenitor cells can be, for example, stem cells that can differentiate into endothelial cells such as hematopoietic stem cells (HSC), HSC endothelial cells, blood vascular endothelial cells, lymph vascular endothelial cells, endothelial cell lines, primary culture endothelial cells, endothelial cells derived from stem cells, bone marrow derived stem cells, cord blood derived cells, human umbilical vein endothelial cells (HUVEC), lymphatic endothelial cells, endothelial progenitor cells, endothelial cell lines, endothelial cells generated from stem cells in vitro, endothelial cells extracted from adipose tissue, smooth muscle cells, interstitial fibroblasts, myofibroblasts, periodontal tissue, tooth pulp, or vascular-derived cells. It is understood that HSC endothelial cells are endothelial cells differentiated from HSCs. Vascular progenitor cells can be isolated from, for example, bone marrow, soft tissue, muscle, tooth, blood and/or vascular system. In some configurations, vascular progenitor cells can be derived from tissue progenitor cells.

Cell densities in a matrix can be monitored over time and at end-points. Tissue properties can be determined, for example, using standard techniques known to skilled artisans, such as histology, structural analysis, immunohistochemistry, biochemical analysis, and mechanical properties. As will be recognized by one skilled in the art, the cell densities of progenitor cells can vary according to, for example, progenitor type, tissue or organ type, matrix material, matrix volume, infusion method, seeding pattern, culture medium, growth factors, incubation time, incubation conditions, and the like. Generally, for progenitor cells, the cell density of each cell type in a matrix can be, independently, from 0.0001 million cells (M) ml⁻¹ to about 1000 M ml⁻¹. For example, the progenitor cells can be present in the matrix at a density of about 0.001 M ml⁻¹, 0.01 M ml⁻¹, 0.1 M ml⁻¹, 1 M ml⁻¹, 5 M ml⁻¹, 10 M ml⁻¹, 15 M ml⁻¹, 20 M ml⁻¹, 25 M ml⁻¹, 30 M ml⁻¹, 35 M ml⁻¹, 40 M ml⁻¹, 45 M ml⁻¹, 50 M ml⁻¹, 55 M ml⁻¹, 60 M ml⁻¹, 65 M ml⁻¹, 70 M ml⁻¹, 75 M ml⁻¹, 80 M ml⁻¹, 85 M ml⁻¹, 90 M ml⁻¹, 95 M ml⁻¹, 100 M ml⁻¹, 200 M ml⁻¹, 300 M ml⁻¹, 400 M ml⁻¹, 500 M ml⁻¹, 600 M ml⁻¹, 700 M ml⁻¹, 800 M ml⁻¹, or 900 M ml⁻¹.

In some embodiments, cells introduced to the matrix can comprise a heterologous nucleic acid so as to express a bioactive molecule such as heterologous protein, or to overexpress an endogenous protein. In non-limiting example, cells introduced to the matrix can express a fluorescent protein marker, such as GFP, EGFP, BFP, CFP, YFP, or RFP. In another example, cells introduced to the matrix can express an angiogenesis-related factor, such as activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin and fibronectin receptor α₅β₁, Factor X, HB-EGF, HBNF, HGF, HUAF, heart derived inhibitor of vascular cell proliferation, IFN-gamma, IL1, IGF-2 IFN-gamma, integrin receptors (e.g., various combinations of a subunits (e.g., α₁, α₂, α₃, α₄, α₅, α₆, α₇, α₈, α₉, α_{E}, α_{V}, α_{IIb}, α_{L}, α_{M}, α_{X}), K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-beta, Tie 1, Tie2, TGF-β, and TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF.sub.164, VEGI, EG-VEGF, VEGF receptors, PF4, 16 kDa fragment of prolactin, prostaglandins E1 and E2, steroids, heparin, 1-butyryl glycerol (monobutyrin), or nicotinic amide. As another example, cells introduced to a matrix can comprise genetic sequences that reduce or eliminate an immune response in the host (e.g., by suppressing expression of cell surface antigens such as class I and class II histocompatibility antigen).

In some embodiments, one or more cell types in addition to a first type of cell can be introduced into or onto the matrix material. Such additional cell type can be selected from those discussed above, or can include (but not limited to) skin cells, liver cells, heart cells, kidney cells, pancreatic cells, lung cells, bladder cells, stomach cells, intestinal cells, cells of the urogenital tract, breast cells, skeletal muscle cells, skin cells, bone cells, cartilage cells, keratinocytes, hepatocytes, gastro-intestinal cells, epithelial cells, endothelial cells, mammary cells, skeletal muscle cells, smooth muscle cells, parenchymal cells, osteoclasts, or chondrocytes. These cell-types can be introduced prior to, during, or after vascularization of the matrix. Such introduction can take place in vitro or in vivo, or a combination thereof. When cells are introduced in vivo, the introduction can be at the site of the engineered vascularized tissue or organ composition or at a site removed there from. Exemplary routes of administration of the cells include injection and surgical implantation.

Added Drugs and/or Diagnostics

In some embodiments, the methods and compositions of the present disclosure further comprise additional agents introduced into or onto the matrix. Various agents that can be introduced include, but are not limited to, bioactive molecules, biologic drugs, diagnostic agents, and strengthening agents.

A matrix can further comprise a bioactive molecule. The cells of the matrix can be, for example, genetically engineered to express the bioactive molecule or the bioactive molecule can be added to the matrix. The matrix can also be cultured in the presence of the bioactive molecule. The bioactive molecule can be added prior to, during, or after cells are introduced to the matrix. Non-limiting examples of bioactive molecules include activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin, fibronectin receptor α₅β₁, Factor X, HB-EGF, HBNF, HGF, HUAF, heart derived inhibitor of vascular cell proliferation, IFN-gamma, IL1, IGF-2 IFN-gamma, integrin receptors (e.g., various combinations of a subunits (e.g., α₁, α₂, α₃, α₄, α₅, α₆, α₇, α₈, α₉, α_{E}, α_{V}, α_{IIb}, α_{L}, α_{M}, α_{X}) and β subunits (e.g., β₁, β₂, β₃, β₄, β₅, β₆, β₇, and β₈)), K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPARγ ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-β, Tie 1, Tie2, TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF₁₆₄, VEGI, EG-VEGF, VEGF receptors, PF4, 16 kDa fragment of prolactin, prostaglandins E1 and E2, steroids, heparin, 1-butyryl glycerol (monobutyrin), or nicotinic amide. In other preferred embodiments, the matrix can include a chemotherapeutic agent or immunomodulatory molecule. Such agents and molecules are known to the skilled artisan.

Biologic drugs that can be added to the compositions of the invention include immunomodulators and other biological response modifiers. A biological response modifier generally encompasses a biomolecule (e.g., peptide, peptide fragment, polysaccharide, lipid, antibody) that is involved in modifying a biological response, such as the immune response or tissue or organ growth and repair, in a manner which enhances a particular desired therapeutic effect, for example, the cytolysis of bacterial cells or the growth of tissue- or organ-specific cells or vascularization. Biologic drugs can also be incorporated directly into the matrix component. Those of skill in the art will know, or can readily ascertain, other substances which can act as suitable non-biologic and biologic drugs.

Compositions can also be modified to incorporate a diagnostic agent, such as a radiopaque agent. The presence of such agents can allow a physician to monitor the progression of wound healing occurring internally. Such compounds include barium sulfate as well as various organic compounds containing iodine. Examples of these latter compounds include iocetamic acid, iodipamide, iodoxamate meglumine, iopanoic acid, as well as diatrizoate derivatives, such as diatrizoate sodium. Other contrast agents which can be utilized in the compositions of the invention can be readily ascertained by those of skill in the art and may include the use of radiolabeled fatty acids or analogs thereof.

Concentration of an agent in the composition will vary with the nature of the compound, its physiological role, and desired therapeutic or diagnostic effect. A therapeutically effective amount is generally a sufficient concentration of therapeutic agent to display the desired effect without undue toxicity. A diagnostically effective amount is generally a concentration of diagnostic agent which is effective in allowing the monitoring of the integration of the tissue graft, while minimizing potential toxicity. In any event, the desired concentration in a particular instance for a particular compound is readily ascertainable by one of skill in the art.

A matrix composition can be enhanced, or strengthened, through the use of such supplements as human serum albumin (HSA), hydroxyethyl starch, dextran, or combinations thereof. The solubility of the matrix compositions can also be enhanced by the addition of a nondenaturing nonionic detergent, such as polysorbate 80. Suitable concentrations of these compounds for use in the compositions of the invention will be known to those of skill in the art, or can be readily ascertained without undue experimentation. The matrix compositions can also be further enhanced by the use of optional stabilizers or diluent. The proper use of these would be known to one of skill in the art, or can be readily ascertained without undue experimentation.

Method of Treatment

A scaffold described herein holds significant clinical value because of the increased capacity for vascularization, as compared to other conventional engineered constructs. It is this increase in vascularization, enabling more efficient regeneration of tissue or organ or better integration of a medical device, which sets the present scaffolds apart from other conventional treatment options.

Also provided is a process of treating tissue or organ defect in a subject in need administration of a therapeutically effective amount of scaffold containing one or more compositions that promote an M1 macrophage phenotype or an M2 macrophage phenotype, so as to increase vascularization of the scaffold, e.g., when implanted.

Generally, a safe and effective amount of scaffold is, for example, that amount that would cause the desired therapeutic effect in a subject while minimizing undesired side effects. Compositions included in or on the scaffold can be present in a therapeutically effective amount and employed in pure form or, where such forms exist, in pharmaceutically acceptable salt form and with or without a pharmaceutically acceptable excipient. For example, compositions of the present disclosure can be administered, at a reasonable benefit/risk ratio applicable to any medical treatment, in a sufficient amount to increase vascularization of a scaffold

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Methods described herein are generally performed on a subject in need thereof. A subject in need of the therapeutic methods described herein can be a subject having, diagnosed with, suspected of having, or at risk for developing a tissue or organ defect. A determination of the need for treatment will typically be assessed by a history and physical exam consistent with the disease or condition at issue. Subjects with an identified need of therapy include those with a diagnosed tissue or organ defect. Diagnosis of the various conditions treatable by the methods described herein is within the skill of the art. The subject is preferably an animal, including, but not limited to, mammals, reptiles, and avians, such as horses, cows, dogs, cats, sheep, pigs, mice, rats, monkeys, hamsters, guinea pigs, and chickens, or humans.

As an example, a subject in need may have a deficiency of at least 5%, 10%, 25%, 50%, 75%, 90% or more of a particular cell type. As another example, a subject in need may have damage to a tissue or organ, and the method provides an increase in biological function of the tissue or organ by at least 5%, 10%, 25%, 50%, 75%, 90%, 100%, or 200%, or even by as much as 300%, 400%, or 500%. As yet another example, the subject in need may have a disease, disorder, or condition, and the method provides an engineered tissue or organ construct sufficient to ameliorate or stabilize the disease, disorder, or condition. For example, the subject may have a disease, disorder, or condition that results in the loss, atrophy, dysfunction, or death of cells. Exemplary treated conditions include a neural, glial, or muscle degenerative disorder, muscular atrophy or dystrophy, heart disease such as congenital heart failure, hepatitis or cirrhosis of the liver, an autoimmune disorder, diabetes, cancer, a congenital defect that results in the absence of a tissue or organ, or a disease, disorder, or condition that requires the removal of a tissue or organ, ischemic diseases such as angina pectoris, myocardial infarction and ischemic limb, accidental tissue defect or damage such as fracture or wound. In a further example, the subject in need may have an increased risk of developing a disease, disorder, or condition that is delayed or prevented by the method.

Implantation of a scaffold is within the skill of the art. In some embodiments, a scaffold described herein can be placed in fluid communication with cells of a subject in vitro or in vivo. As used herein, a scaffold is in “fluid communication” with a cell if the cell has no physical barrier (e.g., a basement membrane, areolar connective tissue, adipose connective tissue, etc.) preventing the cell from migrating to the scaffold.

The scaffold can be either fully or partially implanted into a tissue or organ of the subject to become a functioning part thereof. An implanted scaffold can initially attach to and communicate with the host through a cellular monolayer. Over time, cells can colonize, migrate, or expand into or through the scaffold or introduced cells can expand and migrate out of the scaffold to the surrounding tissue. After implantation, cells surrounding the scaffold can enter through cell migration. The cells surrounding the scaffold can be attracted by biologically active materials, including biological response modifiers, such as polysaccharides, proteins, peptides, genes, antigens, or antibodies that can be selectively incorporated into the matrix to provide the needed selectivity, for example, to tether the cell receptors to the matrix or stimulate cell migration into the matrix, or both. Generally, the matrix is porous, having interconnecting channels that allow for cell migration, augmented by both biological and physical-chemical gradients. One of skill in the art will recognize and know how to use biologically active materials that are appropriate for attracting cells to the matrix.

Administration of compositions or scaffold comprising compositions described herein can occur as a single event or over a time course of treatment. For example, administration can be daily, weekly, bi-weekly, or monthly.

Treatment in accord with the methods described herein can be performed prior to, concurrent with, or after conventional treatment modalities for the disease or condition (e.g., tissue or organ defect). Compositions or scaffold comprising compositions described herein can be administered simultaneously or sequentially with another agent, such as an antibiotic, an antiinflammatory, or another agent. For example, a administration can occur simultaneously with another agent, such as an antibiotic or an anti-inflammatory.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to scaffolds or compositions described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. Similarly, recitation of discrete values is intended to also serve as a shorthand method for referring to a ranges between each recited value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Monocyte Isolation and Preparation of Polarized Macrophages

Monocytes were isolated from the peripheral human blood using sequential density gradient centrifugations of Ficoll and Percoll (see Danciger 2004 Journal of immunological methods 288(1-2):123-134). The yield of CD14+ monocytes, assessed by flow cytometry, was typically around 70%. The monocytes were cultured in ultra low attachment flasks in RPMI 1640 medium with 10% heat-inactivated human serum and 20 ng/ml monocyte colony stimulating factor (MCSF) to differentiate them into macrophages. Medium was changed at day 3. By day 5, the macrophages were attached to the plastic. Polarization was begun by changing to fresh medium supplemented with 20 ng/ml MCSF and the following cytokines: 100 ng/mL interferon-γ (IFNγ) and 100 ng/mL lipopolysaccharide (LPS) for M1; 40 ng/mL IL4 and 20 ng/mL IL13 for M2a; and 40 ng/mL IL10 for M2c. After 48 hrs of polarization, macrophages were collected by gentle scraping, a small sample was taken for RTPCR analysis, and the rest of the cells were incubated in fresh medium at 1 million cells/mL with no cytokines for 24 hrs. Macrophages were collected again by scraping and analyzed by flow cytometry, and the conditioned medium was collected, centrifuged at 400 g for 10 min, and frozen at −80 C until use.

LPS Contamination.

Medium was tested for LPS contamination using the Pierce LAL Chromogenic Endotoxin Quantification kit according to manufacture's instructions. LPS contamination was <0.2 EU/mL.

Flow Cytometric Analysis.

The expression of surface antigens was evaluated by incubating 125,000 M0, M1, M2a or M2c macrophages at 4° C. for 1 hour with the respective antibodies in 100 μL FACS buffer (1 mM EDTA in PBS with 0.5% BSA) (Sigma). The molecules evaluated were antigen-presenting molecule HLA-DR, chemokine receptor CCR7 and scavenger receptors CD163 and CD206. The following antibodies were used for evaluation: FITC-conjugated mouse antibodies against CD206 (Biolegend.com, catalog no. 321103, dilution 1:100), APC-conjugated mouse antibodies against CD163 (Abcam, catalog no. ab134416, dilution 1:50) and CCR7 (Biolegend.com, catalog no. 353213, dilution 1:50), and PE-conjugated mouse antibodies against HLA-DR (Abcam, catalog no. ab113839, dilution 1:100). Corresponding isotype controls were used as recommended by the manufacturers for comparison with each antibody. Labeled cells were washed twice in 1 mL FACS buffer and fixed using Cytofix (BD Pharmingen). The samples were analyzed using a FACSCalibur flow cytometer and the CellQuest software (BD Biosciences, PharMingen). Data was processed using FlowJo (Tree Star) and the percentage population of each cell type stained positive for the respective markers was compared by gating at 1% inclusion of isotype controls to eliminate non-specific staining.

RNA Extraction and cDNA Synthesis.

RNAqueous®-Micro kit (Life Technologies) for RNA extraction was used according to the manufacturer's instructions, eluting the samples at the final step with 5 μL of elution solutions three times. The quantity of RNA was measured on a Nanodrop ND1000 and considered pure if the 260/280 wavelength value was 2. The samples were then stored at −80° C. until used for reverse transcription. The RNA was first treated with DNase I removal kit (Invitrogen) according to the manufacturer's instructions. cDNA synthesis was preformed using High Capacity kit from Applied Biosystems according to the manufacturer's instructions. Each reaction tube contained 1000 ng RNA.

Quantitative of Analysis of Gene Expression Using RT-PCR.

Quantitative RT-PCR analysis was performed using 20 ng cDNA per reaction and the SYBR® Green PCR Master Mix (Applied Biosystems by Life Technologies). The expression of target genes was normalized to the housekeeping gene GAPDH, and then to the unactivated M0 phenotype (2^{-ΔΔCt}). Gene expression values were calculated by using the mean C_{T} values of the samples. All primers (TABLE 1) were synthesized by Life Technologies.

TABLE 1
Table of genes examined and their primer sequences.

| Gene | Forward Sequence | Reverse Sequence |
|---|---|---|
| CCL18 | GCTCTCTGCCCGTCTATACC | GGGCTGGTTTCAGAATAGTCAACT |
| | (SEQ ID NO: 1) | (SEQ ID NO: 2) |
| CCR7 | TGAGGTCACGGACGATTACAT | GTAGGCCCACGAAACAAATGAT |
| | (SEQ ID NO: 3) | (SEQ ID NO: 4) |
| CD163 | TTTGTCAACTTGAGTCCCTTCAC | TCCCGCTACACTTGTTTTCAC |
| | (SEQ ID NO: 5) | (SEQ ID NO: 6) |
| CD206 | AAGGCGGTGACCTCACAAG | AAAGTCCAATTCCTCGATGGTG |
| | (SEQ ID NO: 7) | (SEQ ID NO: 8) |
| CD80 | AAACTCGCATCTACTGGCAAA | GGTTCTTGTACTCGGGCCATA |
| | (SEQ ID NO: 9) | (SEQ ID NO: 10) |
| bFGF | AGAAGAGCGACCCTCACATCA | CGGTTAGCACACACTCCTTTG |
| | (SEQ ID NO: 11) | (SEQ ID NO: 12) |
| GAPDH | AAGGTGAAGGTCGGAGTCAAC | GGGGTCATTGATGGCAACAATA |
| | (SEQ ID NO: 13) | (SEQ ID NO: 14) |
| HBEGF | ATCGTGGGGCTTCTCATGTTT | TTAGTCATGCCCAACTTCACTTT |
| | (SEQ ID NO: 15) | (SEQ ID NO: 16) |
| HLADR | AGTCCCTGTGCTAGGATTTTTCA | ACATAAACTCGCCTGATTGGTC |
| | (SEQ ID NO: 17) | (SEQ ID NO: 18) |
| IL1β | ATGATGGCTTATTACAGTGGCAA | GTCGGAGATTCGTAGCTGGA |
| | (SEQ ID NO: 19) | (SEQ ID NO: 20) |
| IL8 | ACTGAGAGTGATTGAGAGTGGAC | AACCTCTGCACCCAGTTTTC |
| | (SEQ ID NO: 21) | (SEQ ID NO: 22) |
| MDC | GCGTGGTGTTGCTAACCTTCA | AAGGCCACGGTCATCAGAGT |
| | (SEQ ID NO: 23) | (SEQ ID NO: 24) |
| MMP9 | GTACTCGACCTGTACCAGCG | TCAGGGCGAGGACCATAGAG |
| | (SEQ ID NO: 25) | (SEQ ID NO: 26) |
| PDGFB | CTCGATCCGCTCCTTTGATGA | CGTTGGTGCGGTCTATGAG |
| | (SEQ ID NO: 27) | (SEQ ID NO: 28) |
| RANTES | GCCCACATCAAGGAGTATTTCTACA | CGGTTCTTTCGGGTGACAA |
| | (SEQ ID NO: 29) | (SEQ ID NO: 30) |
| TIMP3 | ACCGAGGCTTCACCAAGATG | CATCATAGACGCGACCTGTCA |
| | (SEQ ID NO: 31) | (SEQ ID NO: 32) |
| TNFα | CCTCTCTCTAATCAGCCCTCTG | GAGGACCTGGGAGTAGATGAG |
| | (SEQ ID NO: 33) | (SEQ ID NO: 34) |
| VEGF | AGGGCAGAATCATCACGAAGT | AGGGTCTCGATTGGATGGCA |
| | (SEQ ID NO: 35) | (SEQ ID NO: 36) |

Secreted Protein Quantification Using ELISA.

Human VEGF and PDGF-BB Mini ELISA Development Kits (Peprotech) and MMP9 Quantikine ELISA (R&D Systems) were used according to the manufacturer's instructions.

Gel Zymography.

Conditioned media was assessed for enzymatically active MMP9 content using gel zymography (NovexZymogram gels, Life Technologies). 5 μL of conditioned medium was loaded into the 10% Zymogram (gelatin) gel and run for 90 min at 120V. The gel was developed overnight and stained with SimplyBlue.

Endothelial Cell Isolation and Culture.

Human umbilical cord derived endothelial cells (HUVECs) were isolated from fresh umbilical veins from the neonatal unit at Columbia University following an approved IRB protocol (IRBAAAC4839) according to previously described methods (see Baudin et al. 2007 Nature protocols 2(3), 481-485). HUVECs were cultured in endothelial growth media (EGM2, Lonza) and only cells from passage 2-4 were used in experiments.

In Vitro Sprout Formation Analysis.

Transparent hanging transwell inserts (Millipore, 0.4 μm pore size) were coated in 40 μL of a Matrigel® and endothelial basal media (EBM2) solution (1:1 dilution) and incubated for one hour at 37 C. Each insert was placed in a 24-well plate containing 400 μL of macrophage-conditioned media with an additional 100 μL added directly into each insert (n=3-5 replicates per phenotype per donor, n=2 donors). RPMI media with 10% heat inactivated human serum and EGM2 were used as negative and positive controls, respectively. 20,000 HUVECs were added to each insert and were cultured at 37 C for 18 hours. The cells were then stained with a Live/Dead® kit (Invitrogen) following the manufacturer's instructions and the networks were imaged with the 10× objective of an Olympus IX81 microscope. Calcein-AM was used to indicate live cells, and ethidium homodimer-1 was used to indicate dead cells. Two or three images of each sample were required to capture all sprouts in the samples. Background was removed and the networks were analyzed as described in FIG. 8. Briefly, the images were stitched together using the pairwise and grid/collection stitching toolbox in FIJI (see Preibisch et al. 2009 Bioinformatics 25(11), 1463-1465) resulting in one fused image per sample. The fused images were converted into 8-bit tiffs and adjusted for brightness/contrast to distinguish the networks against the background. A custom-designed algorithm run in MATLAB was utilized to remove any noise (i.e., structures not part of the network). Functions from the Image Processing Toolbox in MATLAB were employed to perform the image manipulation. A map of the background was generated and subtracted from the image, resulting in an image with a completely dark field that was converted into a set of binary images with varying gray threshold values. Morphological cleaning, bridging, and closing operations are performed on the images to smooth the edges of the network and maintain connectivity over fine structural elements. The resulting set of images contained the network elements at varying threshold values, allowing for the creation of a single binary image with each element incorporated at an optimal gray threshold. An element-by-element multiplication was performed between this binary image and the original microscope image to yield a final clean image for network analysis. For each sample, the total area of the networks was calculated in MATLAB and the number of sprouts and nodes was determined using the Angiogenesis Analyzer macro in ImageJ (see Carpentier 2012 ImageJ Contribution, Angiogenesis analyzer, ImageJ News 5). To determine the number of sprouts, the Analyzer was set to resolve the number of segments, isolated segments, and branched segments. To determine the number of nodes, the Analyzer was set to locate each junction point.

Viability and Metabolic Assays.

HUVECs were starved overnight in EBM2 with 0.5% fetal bovine serum (FBS) prior to seeding at a density of 5,000 cells per well in 100 μl conditioned media in a 96 well plate (n=9 per group). EGM-2 was used as a positive control and RPMI with 0.5% FBS was used as a negative control. After 18 hours, the wells were washed with PBS and DNA content was quantified using Quant-iT™ PicoGreen® dsDNA Assay kit (Invitrogen) according to the manufacturer's instructions. DNA was quantified using a standard curve prepared using A-phage DNA. Metabolic activity of the cells during the viability study was measured using Alamar Blue® reagent according to the manufacturer's instructions (Life Technologies).

Scaffold Preparation and Subcutaneous Implantation.

Cylindrical disks (7 mm in diameter×2.5 mm thick) were punched from sheets of Avitene™ Ultrafoam™ collagen sponge. Scaffolds were either soaked in PBS (“Collagen”), 0.1% glutaraldehyde in PBS (“Glutaraldehyde-Crosslinked”), or 100 ng/ml LPS (“LPS-coated”) for 4 hr. Then, scaffolds were washed 4 times for 10 min in PBS, and incubated in RPMI medium for 4 days. Glutaraldehyde-crosslinked scaffolds were soaked for an additional 4 hr in 0.1 M glycine to quench any residual glutaraldehyde, and incubated in RPMI medium overnight.

Scaffolds from the above three groups were implanted subcutaneously in C57/BL6 mice for 10 days (one sample per mouse, n=3 mice per group). To eliminate animal to animal differences, one scaffold of the unmodified collagen and one glutaraldehyde-crosslinked scaffolds were implanted into a single mouse (n=3, for a total of n=6 scaffolds for these groups). Mice were anesthetized using 100 mg/kg ketamine and 10 mg/kg xylazine and shaved. A small incision (<1 cm) was made using a scalpel in the central dorsal surface. Blunt forceps were used to create a pocket in the subcutaneous space for the scaffolds. After implantation, wounds were closed with two sutures. Mice were monitored until after recovery from anesthesia and housed for 10 days. No signs of discomfort were observed following surgery throughout the study.

Histological Analysis.

After 10 days, mice were euthanized by CO₂ asphyxiation. An incision was made and the skin was pulled back to expose the scaffolds. Gross view images were taken immediately with an Olympus SZX16 stereomicroscope. The scaffolds and surrounding tissue were excised and fixed in 4% paraformaldehyde overnight. The samples were washed for 6 hr in PBS, incubated in 30% sucrose for 3 days, embedded in OCT (Tissue-Tek, Torrance, TA) and frozen. Samples were sectioned to 5 μm and mounted onto slides for histological evaluation. Tissue structure was examined by staining with hematoxylin and eosin (H&E), which stains nuclei dark blue to black, and cytoplasm and collagen pink. Images of whole tissue sections were obtained using the stitching function of an Olympus FX100 microscope and software.

Immunofluorescence.

Sections were analyzed for three markers of the M1 phenotype (TNFa, iNOS, and CCR7) and three markers of the M2 phenotype (CD206, Arg1, and CD163), along with the pan-macrophage marker F480, using the antibodies and dilutions (see Zhang et al. 2013 Nature Biotechnology 31(6), 553-556) and CD163(1:50) from Santa Cruz Biotechnology. Endothelial cells were stained with rabbit-anti-mouse CD31 (1:50) from Abcam.

Statistical Analysis.

Data are shown as Mean±SEM. Statistical analysis was performed in GraphPad Prism 5.0 using one-way ANOVA with post-hoc Bonferroni analysis. P<0.05 was considered significant.

### Example 2

### Characterization of Polarized Macrophages Monocytes

Methods are according to Example 1 unless otherwise specified.

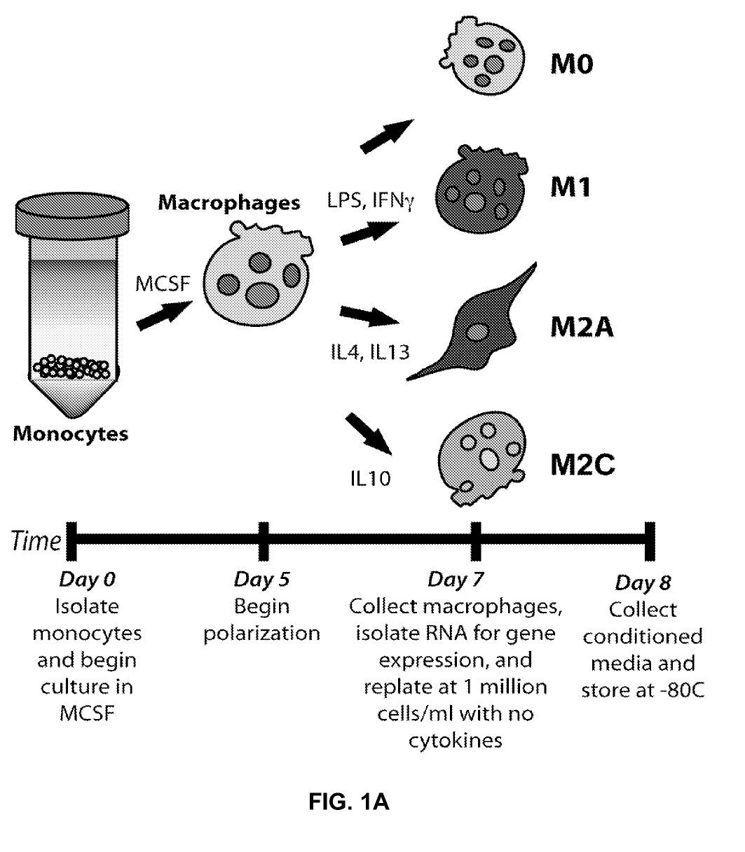

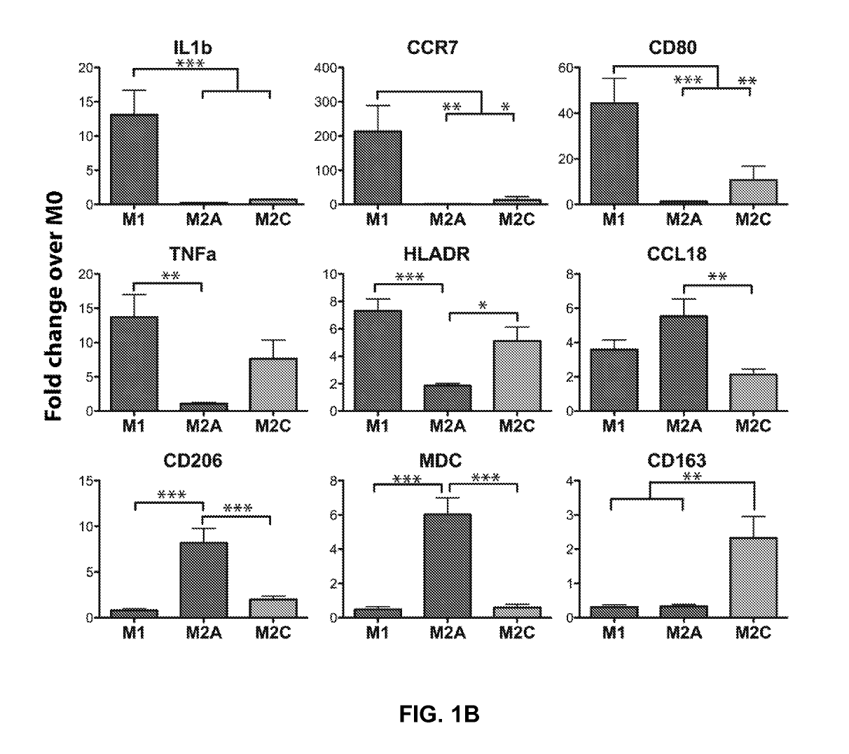

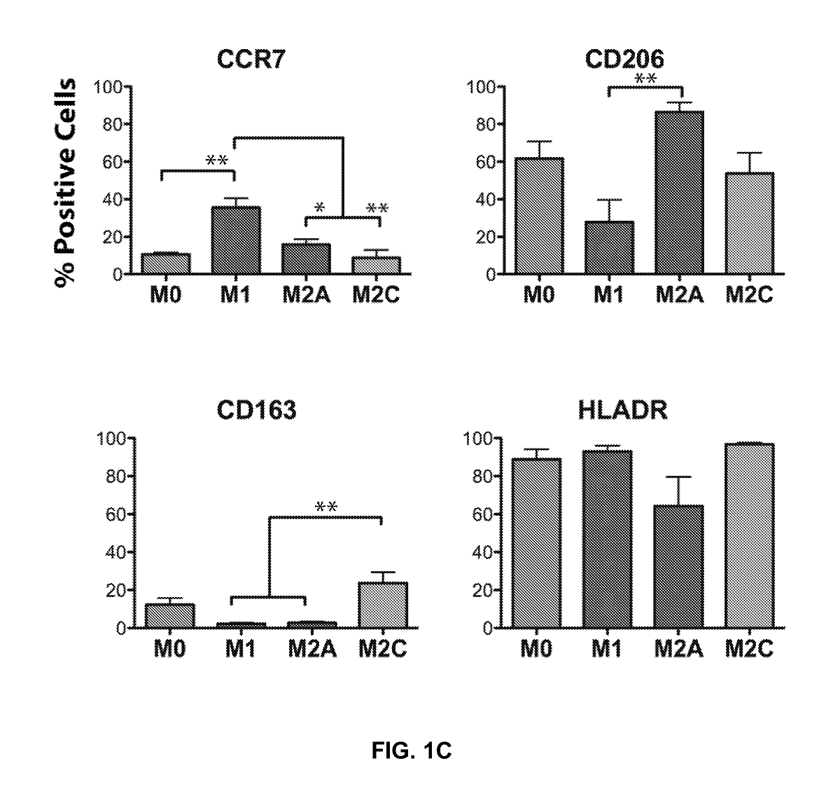

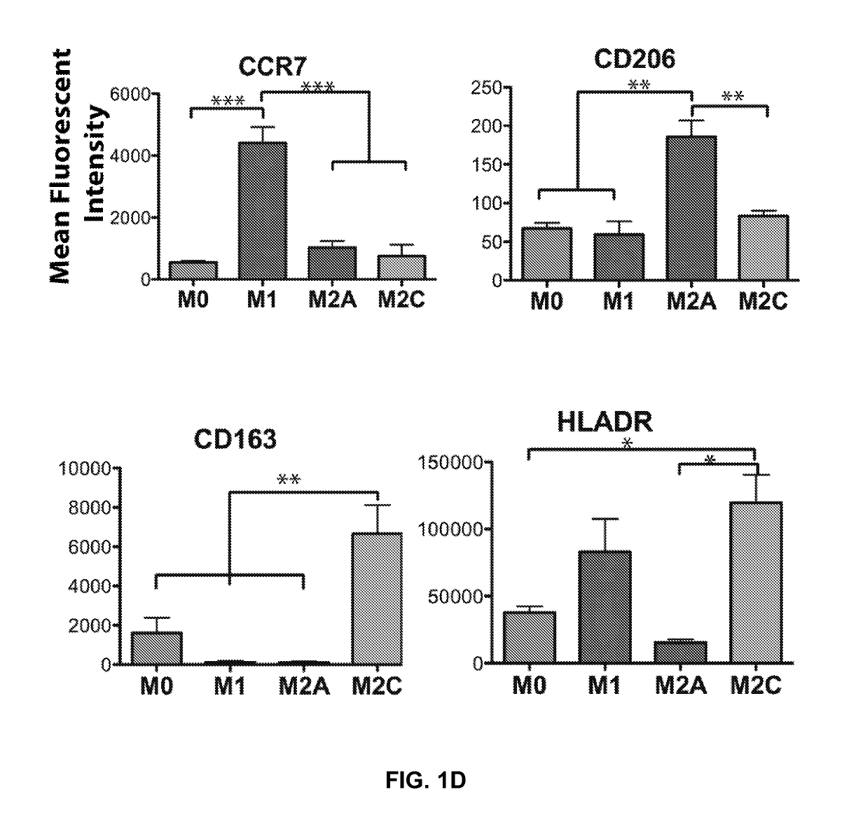

Monocytes isolated from the peripheral human blood were differentiated to macrophages through the addition of monocyte colony stimulating factor (MCSF), and polarized to different macrophage phenotypes via the addition of specific cytokines (see e.g., FIG. 1A). Three phenotypes were prepared (M1, M2a, M2c) and compared to an unactivated control phenotype (M0).

Gene expression analysis revealed that each macrophage phenotype uniquely upregulated specific markers. M1 macrophages strongly upregulated the inflammatory proteins IL18 and tumor necrosis factor-α (TNFα), and the surface markers CCR7, CD80, and HLADR/MHC Class II (see e.g., FIG. 1B). M2a macrophages upregulated the cytokines CCL18 and MDC/CCL22 and the surface marker CD206/mannose receptor. M2c macrophages could be distinguished by expression of the scavenger receptor CD163. M2c macrophages, conventionally considered anti-inflammatory, expressed higher levels of the inflammatory markers TNFα and HLADR than M2a. These levels were lower but not statistically different from M1 macrophages. CD163+ macrophages have been shown in other reports to secrete inflammatory cytokines in response to biomaterials in vitro (see e.g., Bartneck et al. 2012 Biomaterials 33(16), 4136-4146) and in psoriatic skin of patients in vivo (see e.g., Fuentes-Duculan et al. 2010 The Journal of Investigative Dermatology 130(10), 2412-2422).

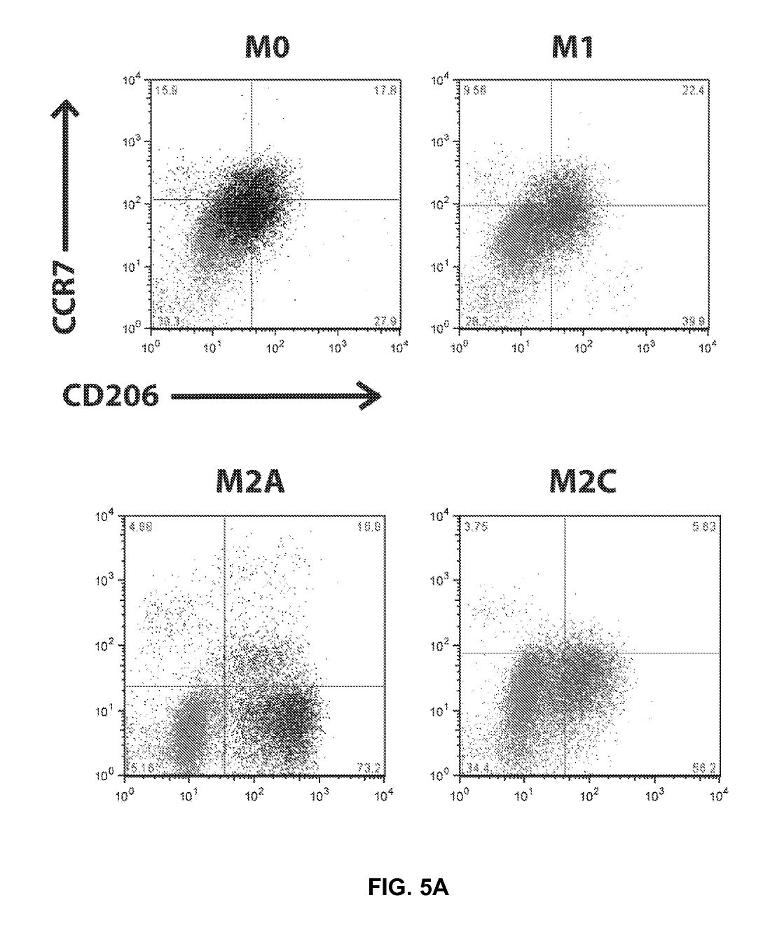

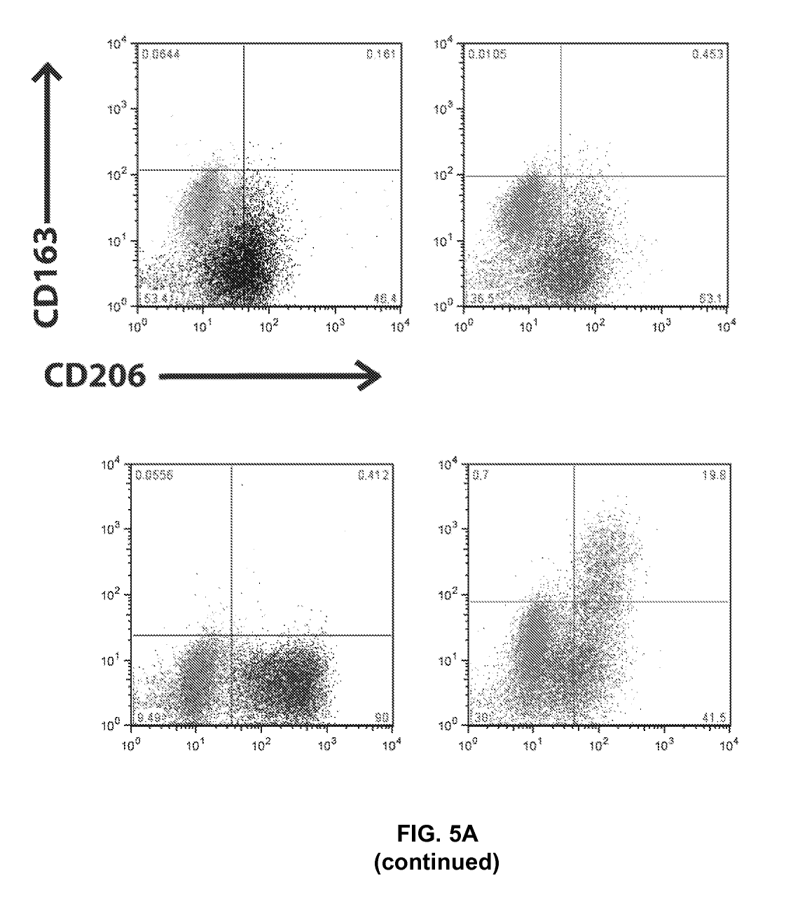

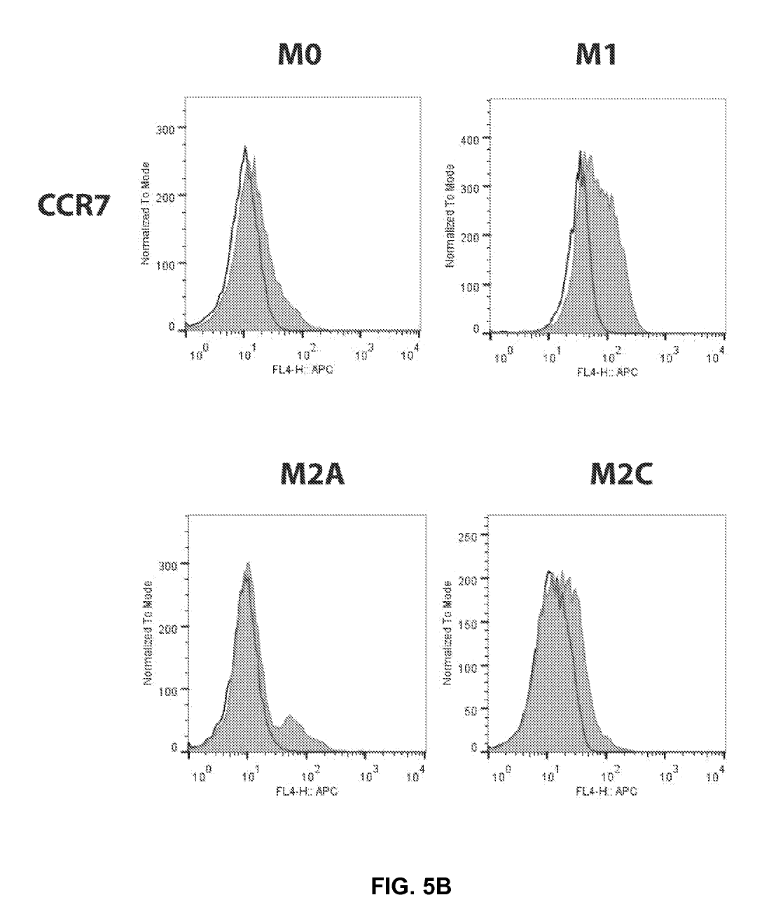

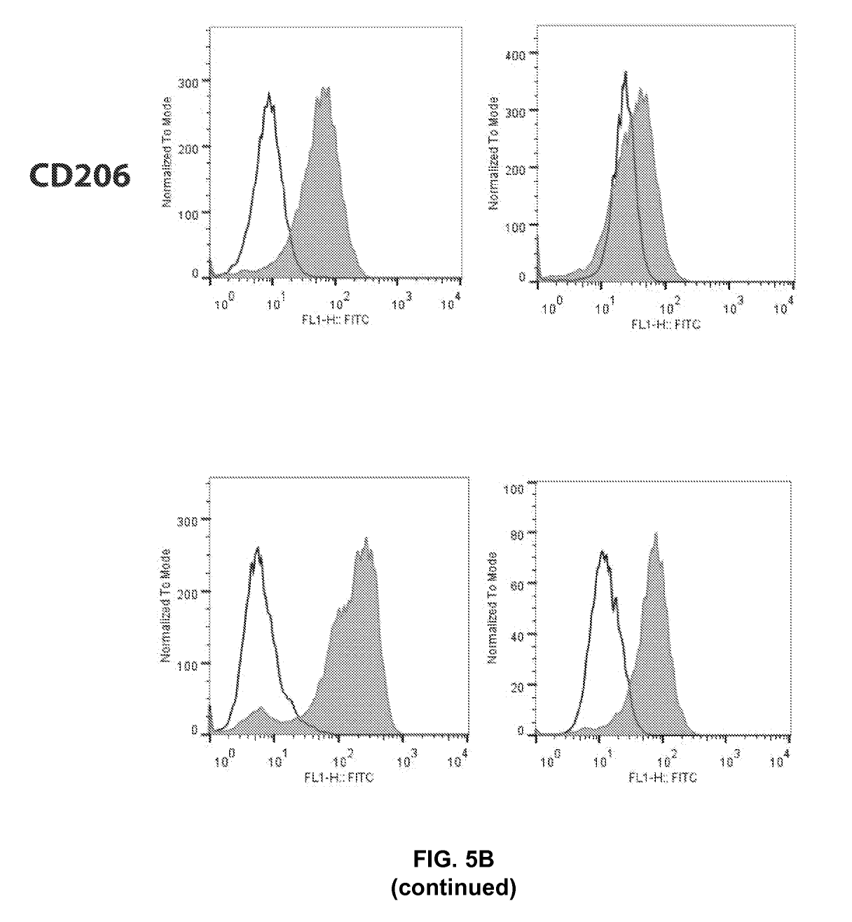

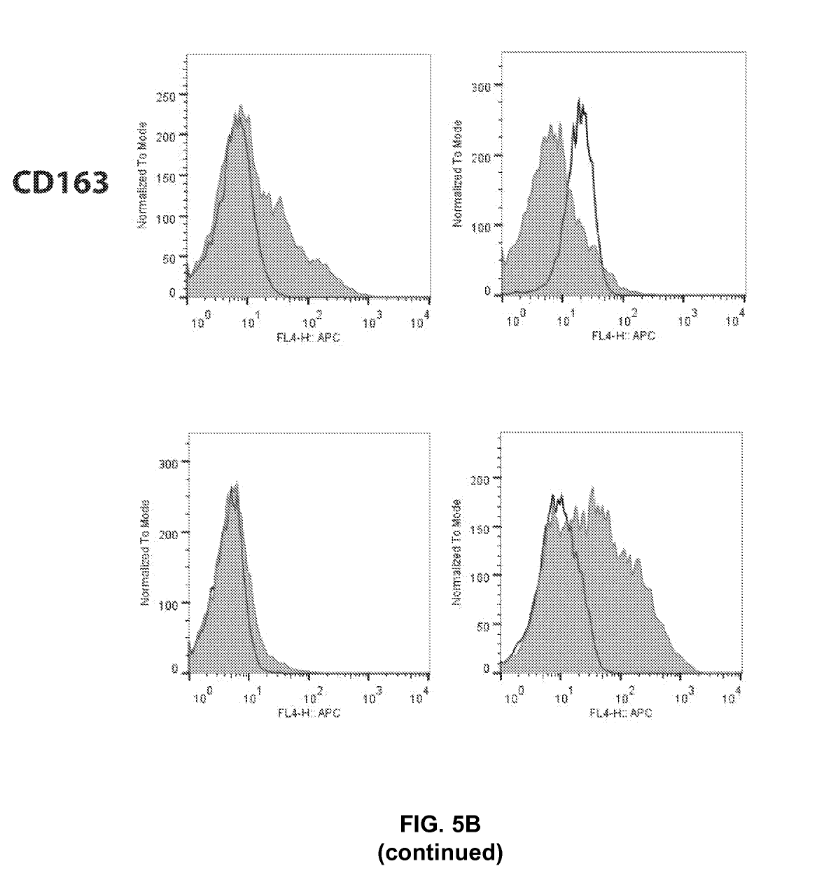

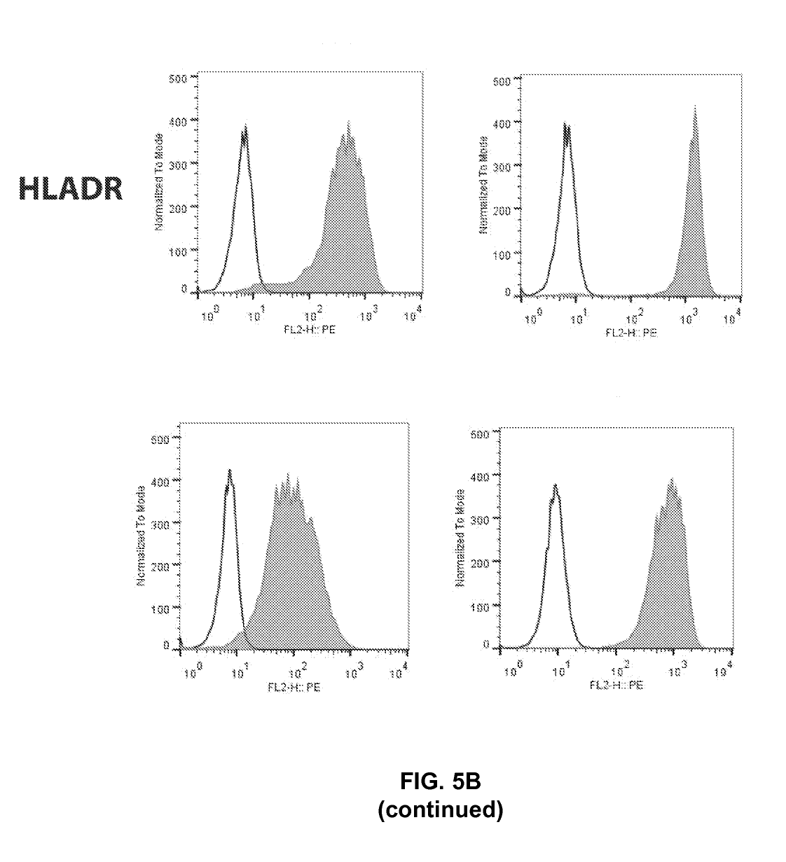

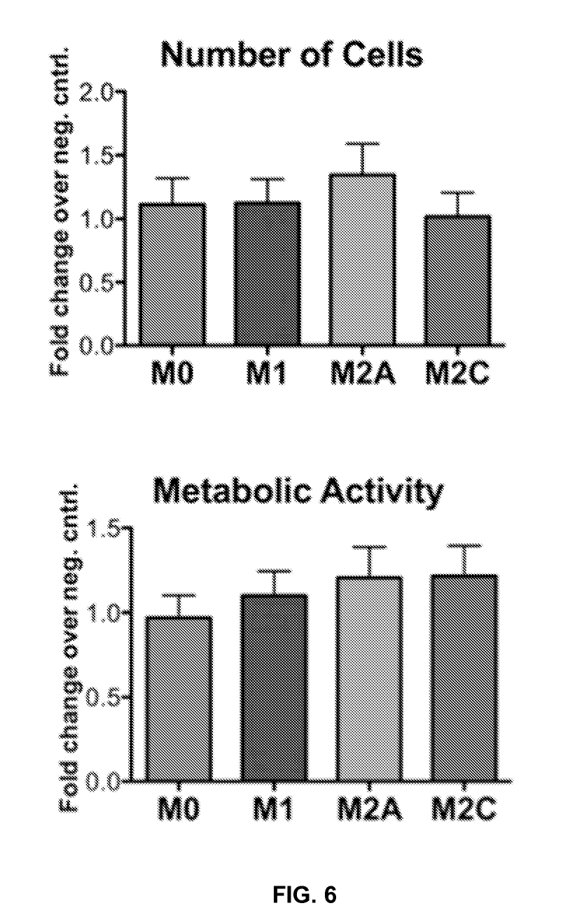

Flow cytometric analysis was used to determine which surface markers would be robust indicators of phenotype. The M1 marker CCR7 was expressed more by M1 macrophages, although expression was still detected on the other phenotypes (see e.g., FIG. 1C). Similarly, CD163 was a good marker of the M2c phenotype, although unactivated M0 macrophages expressed similar levels. Surprisingly, the putative M2a marker CD206 and the M1 marker HLADR were expressed on almost all macrophages of the different phenotypes (see e.g., FIG. 1C). Moreover, a large fraction of CCR7+ cells of each phenotype were also CD206+, and all CD163+ cells were CD206+(see e.g., FIG. 5), indicating that the mere expression of these markers may not be definitive evidence of macrophage phenotype. But mean fluorescent intensity per cell, an indication of how strongly each individual cell expressed the marker, revealed significant differences between the phenotypes (see e.g., FIG. 1D, FIG. 6). Thus, expression above a certain threshold of fluorescence can be used as a phenotype marker.

### Example 3

### Macrophage Phenotype Determines Secretion of Proteins Related to Different Stages of Angiogenesis

Methods are according to Example 1 unless otherwise specified.

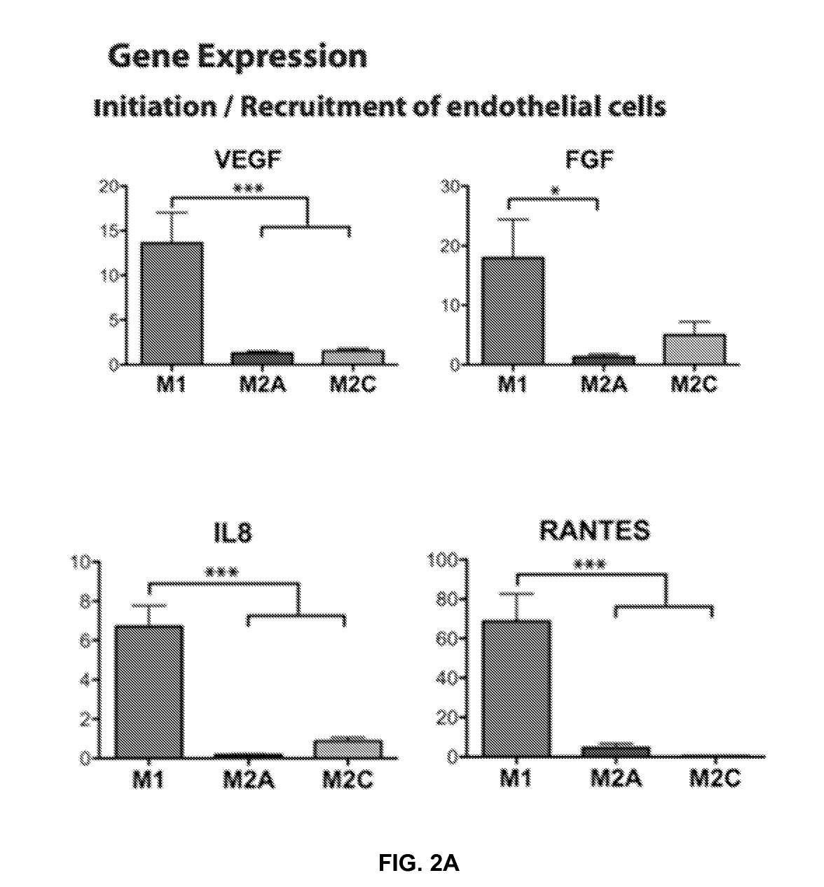

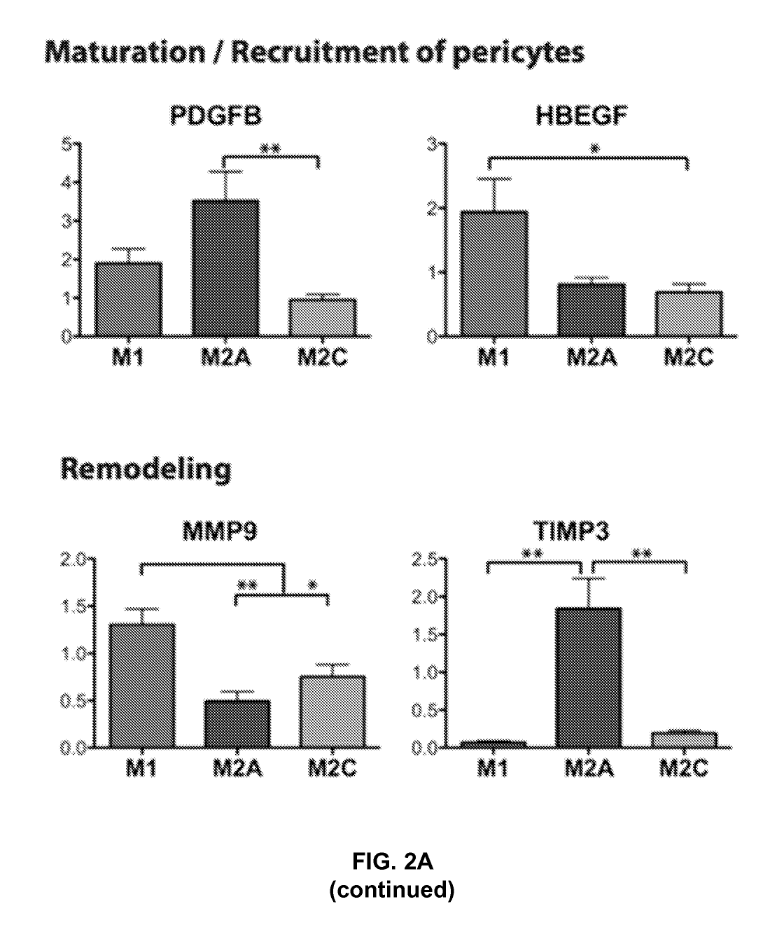

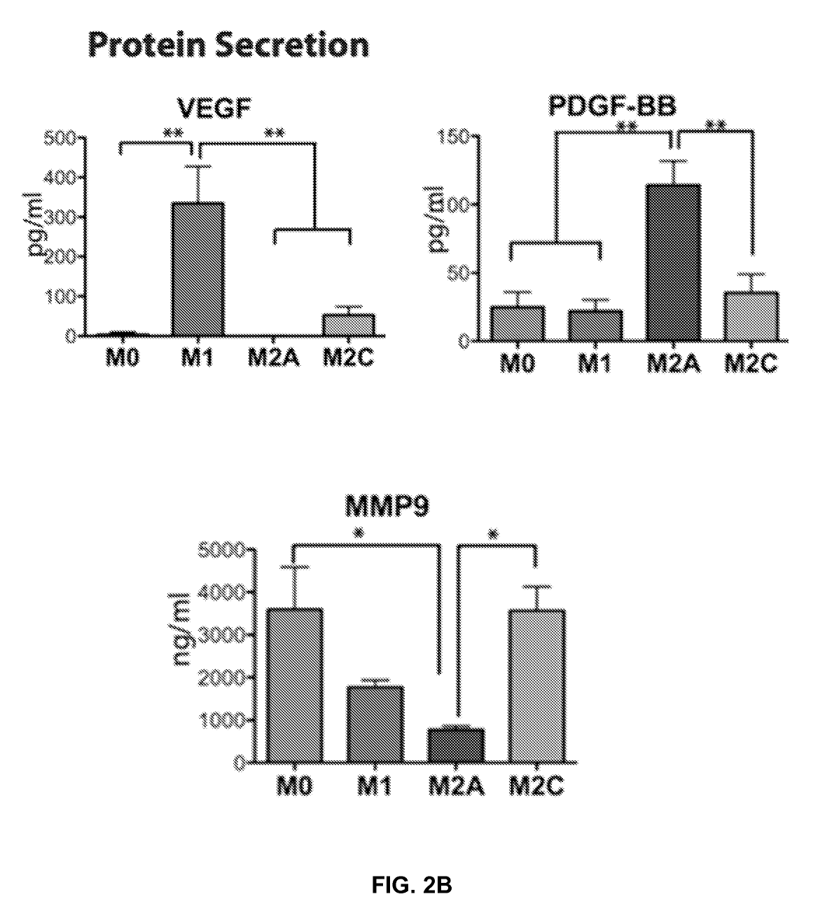

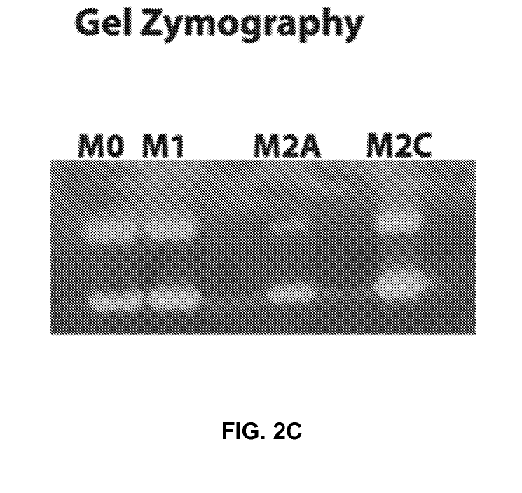

Expression of genes and secretion of proteins involved in angiogenesis was examined (see e.g., FIG. 2A). M1 macrophages expressed genes involved at early stages of angiogenesis, including those that are chemotactic for endothelial cells like VEGF, basic fibroblast growth factor (bFGF), IL8, and RANTES/CCL5 (see e.g., Bartneck 2012 Biomaterials 33(16), 4136-4146; Yoshida et al. 1996 Growth Factors 13(1-2), 57-64; Asahara et al. 1999 The EMBO Journal 18(14), 3964-3972; Martin et al. 2009 The Journal of Biological Chemistry 284(10), 6038-6042; Koch et al. 1992 Science 258(5089), 1798-1801; Suffee et al. 2012 Angiogenesis 15(4), 727-744. Secretion of VEGF was also confirmed on the protein level via enzyme-linked immunosorbent assay (ELISA) (see e.g., FIG. 2B). The inflammatory cytokines TNFα and IL1β, secreted by M1 macrophages, have also been shown to prime endothelial cells for sprouting by increasing the tip cell phenotype (see e.g., Sainson et al. 2008 Blood 111(10), 4997-5007) and to stimulate endothelial cells to recruit supporting pericytes (See e.g., Yoshizumi et al. 1992 Journal of Biological Chemistry 267(14), 9467-9469). Taken together, these results support that M1 macrophages are important initiators of angiogenesis.

M2a macrophages expressed and secreted high levels of PDGF-BB (see e.g., FIG. 2A, FIG. 2B), a factor well known to recruit pericytes that stabilize the growing vasculature (see e.g., Stratman et al. 2010 Blood 116(22), 4720-4730; Hellstron et al. 1999 Development 126(14), 3047-3055) as well as mesenchymal stem cells (see e.g., Ponte et al. 2007 Stem Cells 25(7), 1737-1745). Without this action, VEGF-stimulated blood vessels are leaky, immature, and prone to regression (see e.g., Hellberg et al. 2010 Fortschritte der Krebsforschung. Progres dans les recherches sur le cancer 180, 103-114; Yancopoulos et al. 2000 Nature 407(6801), 242-248). M1 macrophages also expressed high levels of heparin binding EGF-like growth factor (HBEGF) (see e.g., FIG. 2A), suggesting that they can also recruit pericytes. Interestingly, M2a macrophages expressed high levels of tissue inhibitor of matrix metalloprotease-3 (TIMP3) (see e.g., FIG. 2A), which inhibits not only the enzymatic activity of MMP9 but also VEGF signaling by blocking its binding to VEGF receptor 2, resulting in potent inhibition of angiogenesis (see e.g., Qi et al. 2003 Nature medicine 9(4), 407-415). TIMP3 also blocks the release of TNFα (see e.g., Rosenberg 2009 Lancet Neurology 8(2), 205-216). Therefore, M2a macrophages may help support angiogenesis by recruiting pericytes and regulating the signaling of M1 macrophages.

MMP9 is a potent stimulator of angiogenesis in vitro and in vivo, contributing to remodeling of the extracellular matrix in order to allow endothelial cells to migrate, among other functions (see e.g., Jadhav et al. 2004 International Journal of Oncology 25(5), 1407-1414; Ardi et al. 2007 Proceedings of the National Academy of Sciences of the United States of America 104(51), 20262-20267). High levels of MMP9 were secreted by all groups, with M2a macrophages secreting significantly less MMP9 than the other phenotypes (see e.g., FIG. 2B). The MMP9 was confirmed to be enzymatically active by gel zymography (see e.g., FIG. 2C).

### Example 4

### Effects of Macrophage-Conditioned Media on Angiogenesis In Vitro

Methods are according to Example 1 unless otherwise specified.

To confirm the functional role of macrophage-secreted factors in angiogenesis, an in vitro sprouting assay was performed. HUVECs organized into networks with significantly more sprouts and greater total length in M2c-conditioned media compared to HUVECs in media conditioned by M1 or M2a macrophages (see e.g., FIG. 3A). The M2a conditioned medium produced the shortest networks with the least number of sprouts, values that were not statistically different than the base media of RPMI and 10% heat inactivated human serum (see e.g., FIG. 3A). No differences in viability or metabolic activity of HUVECs were found during the experimental time frame (see e.g., FIG. 6). The inhibited sprouting in M2a-conditioned media may be a result of TIMP3 inhibiting MMP9 (see e.g., Rosenberg 2009 Lancet Neurology 8(2), 205-216), which is required for sprouting in vitro (see e.g., Jadhav et al. 2004 International Journal of Oncology 25(5), 1407-1414).

Collectively, macrophage characterization and HUVEC functional assays suggest that all three macrophage phenotypes promote angiogenesis according to the following model: M1 macrophages recruit endothelial cells and initiate angiogenesis via secretion of VEGF; M2a macrophages recruit the stabilizing pericytes via PDGF-B and regulate VEGF and TNFα signaling via TIMP3; and M2c macrophages permit matrix remodeling and blood vessel growth via MMP9 (see e.g., FIG. 3B).

### Example 5

### Both M1 and M2 Macrophages are Required for Vascularization of Tissue Engineering Scaffolds In Vivo

Methods are according to Example 1 unless otherwise specified.

To further confirm the roles of macrophage phenotype for vascularization of biomaterials in vivo, collagen scaffolds designed to elicit a range of macrophage phenotypes were implanted subcutaneously in mice for ten days.

According to conventional understanding, unmodified collagen scaffolds were expected to elicit a primarily M2 response, crosslinked scaffolds were expected to promote a moderate M1 response as described for small intestinal submucosa (see e.g., Badylak et al. 2008 Tissue Engineering Part A 14(11), 1835-1842), and scaffolds coated in LPS were expected to promote a strong M1 response, since LPS is a component of the bacterial cell wall that is frequently used to polarize macrophages to the M1 phenotype.

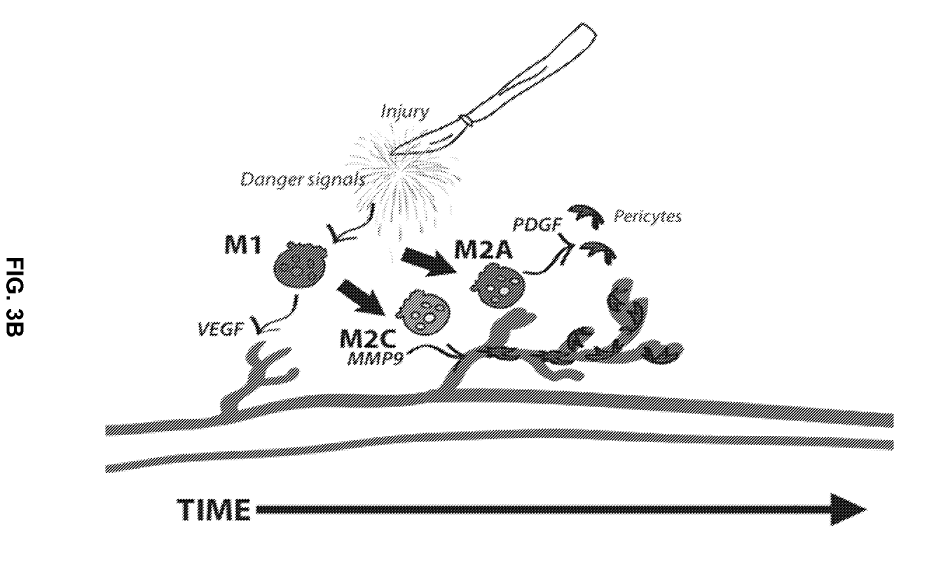

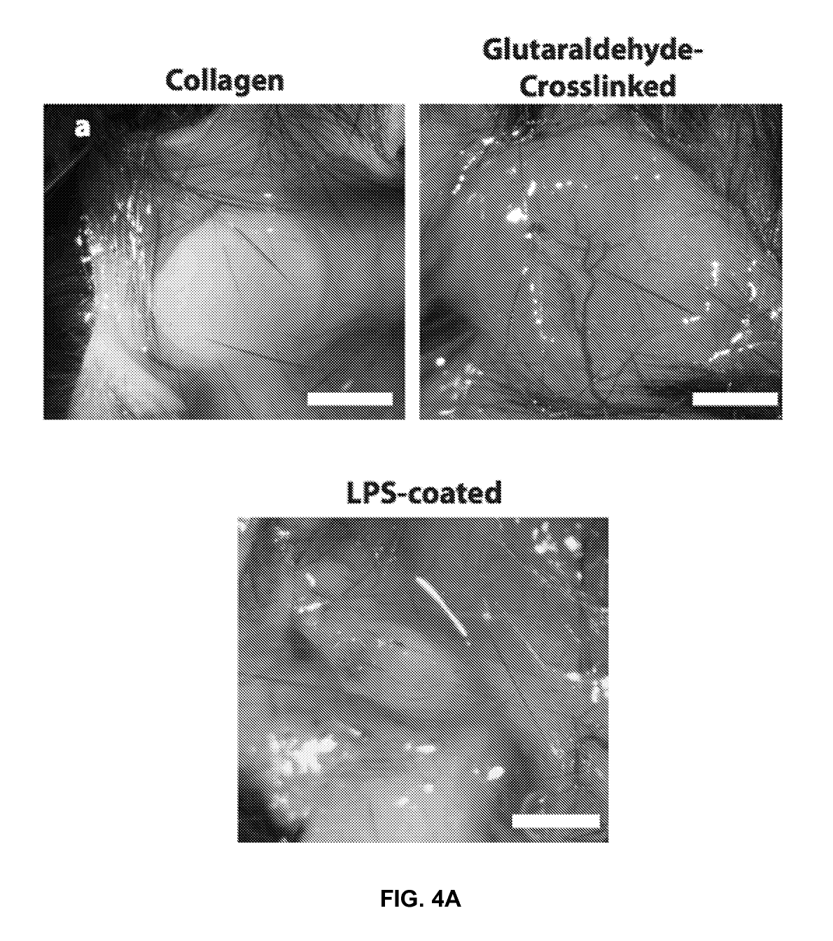

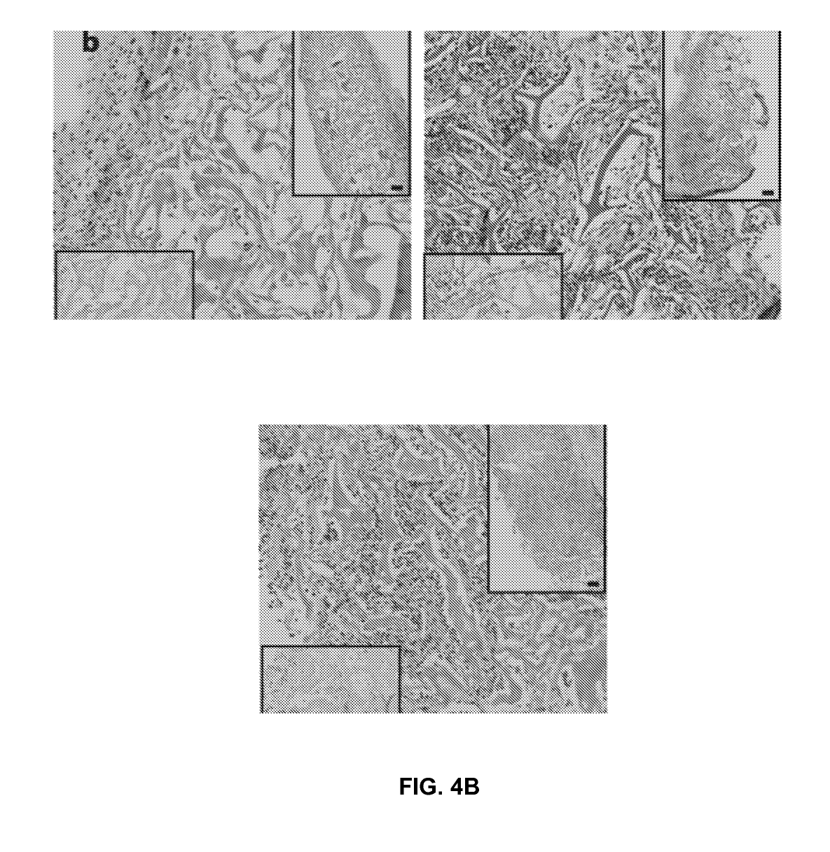

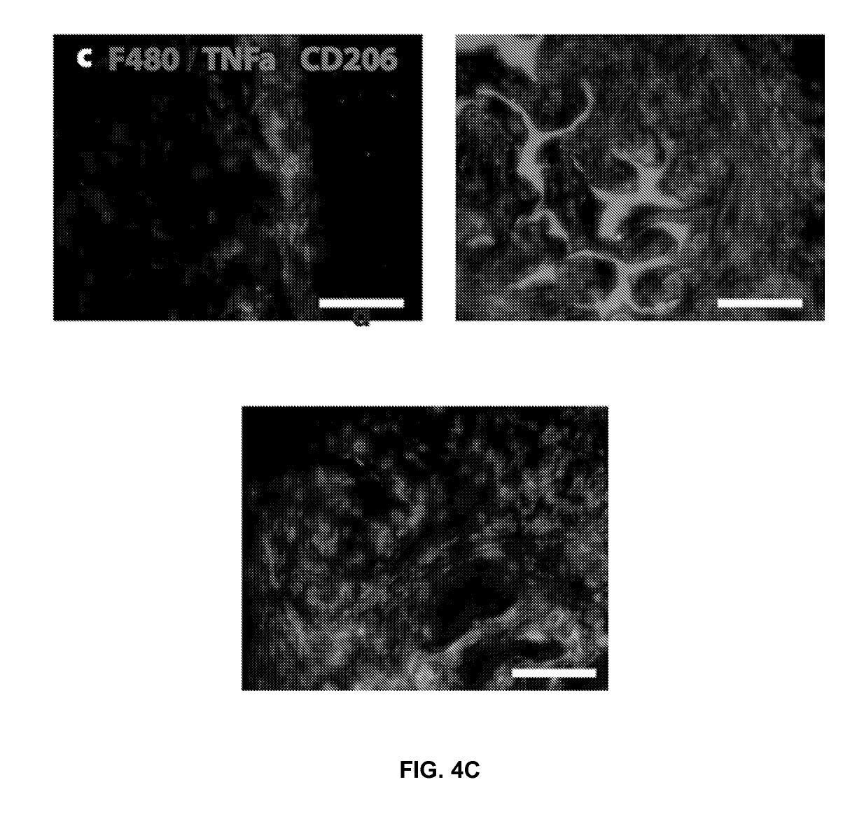

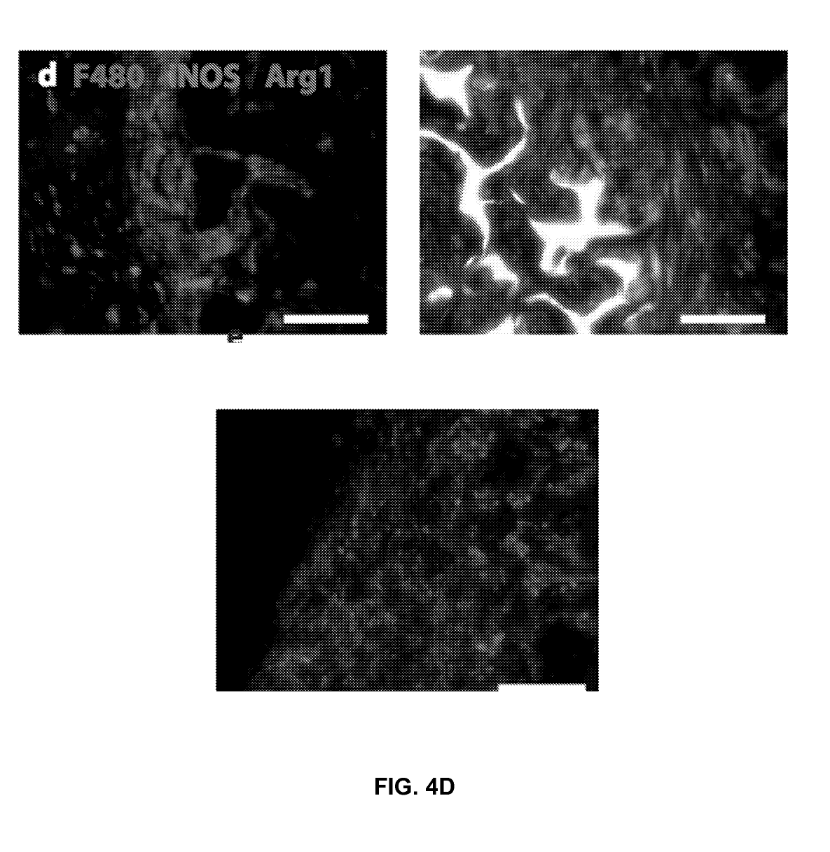

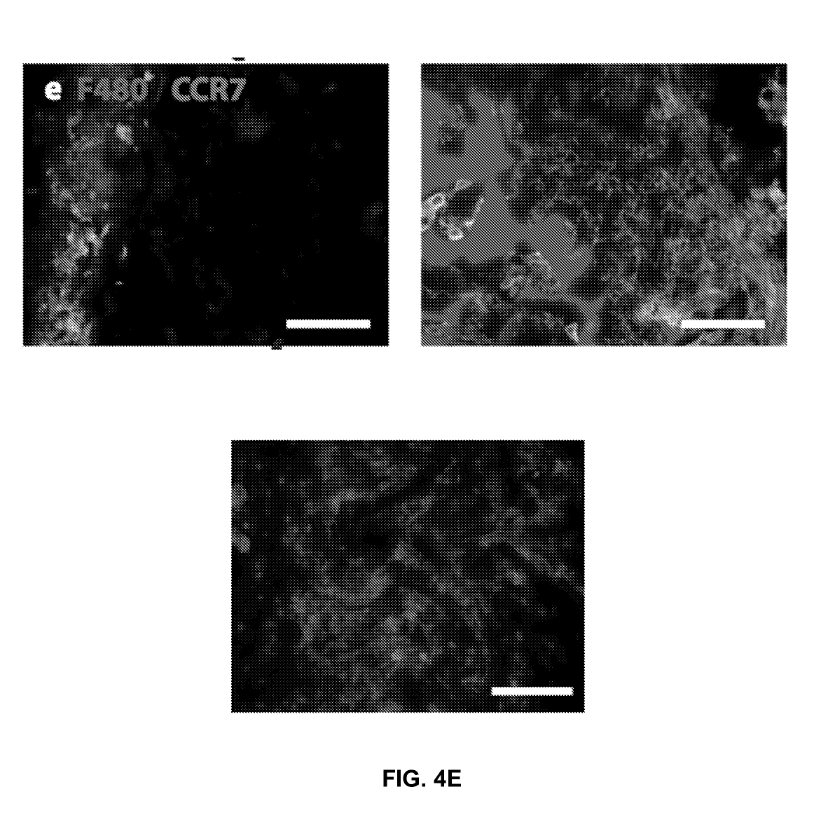

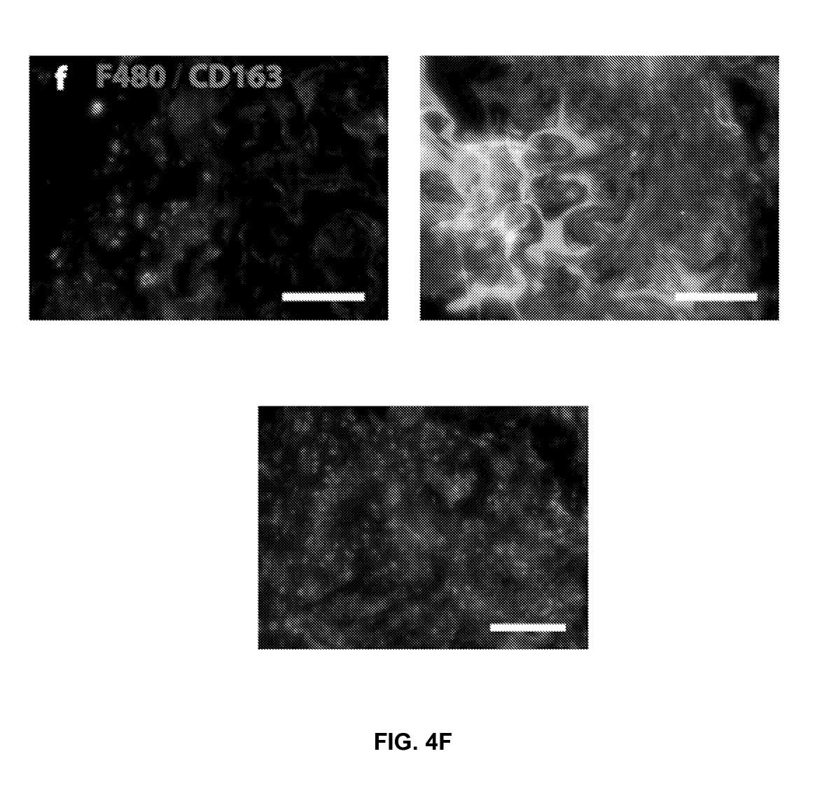

Results showed marked differences in the inflammatory responses of the three scaffold groups 10 days after implantation. A dense fibrous capsule surrounded unmodified collagen scaffolds (see e.g., FIG. 4A, FIG. 4B). No blood vessels were observed in histological sections, and no staining by the endothelial cell marker CD31 could be detected (see e.g., FIG. 4B, insets). In contrast, crosslinked scaffolds were well vascularized, with macroscopically visible blood vessel infiltration (see e.g., FIG. 4A) and histological sections (see e.g., FIG. 4B) and abundant staining by CD31. LPS-coated scaffolds were completely infiltrated by large numbers of inflammatory cells (see e.g., FIG. 4B) with no evidence of blood vessels or endothelial cell staining. Both control and LPS-coated scaffolds were considerably smaller and more degraded than crosslinked scaffolds (see e.g., FIG. 4A).

To identify the macrophage subtypes involved in these different inflammatory responses, sections were stained for multiple markers of macrophage phenotype (see e.g., FIG. 4C, FIG. 4D, FIG. 4E, FIG. 4F). Given that the differences between murine M2a and M2c macrophages have not been systematically characterized, differentiate between M2a and M2c macrophages was not attempted. Instead, traditional M1 and M2 markers were used in combination with the pan-macrophage marker F480 to describe the phenotypes surrounding the scaffolds in vivo.

Both glutaraldehyde-crosslinked and LPS-coated scaffolds were infiltrated by F480+ macrophages, while macrophages remained on the outside of unmodified scaffolds in the fibrous capsule (see e.g., FIG. 4). Due to the large numbers of macrophages surrounding the scaffolds, and that positive staining does not necessarily indicate phenotype (see e.g., FIG. 1C), it was not possible to quantify individual macrophage phenotypes, but qualitative observations are summarized in TABLE 2.

TABLE 2
Qualitative observations for immunofluorescent staining
of macrophage phenotype in explanted scaffolds and
surrounding tissue from in vivo study (see FIG. 4C-F).

| | | Glutaraldehyde- | |
|---|---|---|---|
| Marker | Collagen | Crosslinked | LPS-coated |
| TNFa (M1) | + | +++ | +++ |
| iNOS (M1) | + | +++ | ++ |
| CCR7 (M1) | ++ | ++ | ++ |
| CD206 (M2) | ++ | ++ | ++ |
| Arg1 (M2) | +++ | − | + |
| CD163 (M2) | ++ | +++ | + |

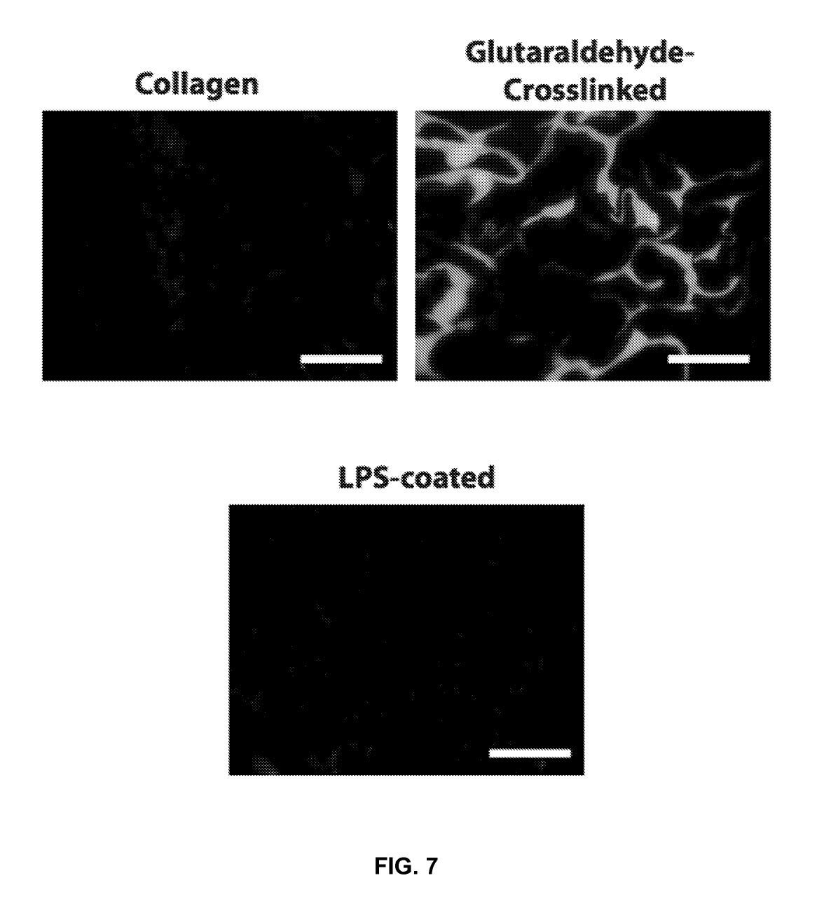

As expected, macrophages surrounding collagen scaffolds stained weakly for the M1 markers TNFα and iNOS and strongly for the M2 markers CD206, Arg1, and CD163. Expression of Arg1 was notably higher than in the other scaffolds. Crosslinked scaffolds stained strongly for all M1 and M2 markers examined except for Arg1. LPS-coated scaffolds stained strongly for the M1 markers TNFa, iNOS, and CCR7, and weakly for the M2 markers Arg1 and CD163. There was no difference in CD206 or CCR7 expression among the groups. Negative control images are shown in FIG. 7.

These findings extend the above described in vitro findings to an in vivo mouse model and further support using diverse macrophage responses for achieving robust vascularization.

### Example 6

### Scaffold with Attached IL4 and Physically Adsorbed IFNy

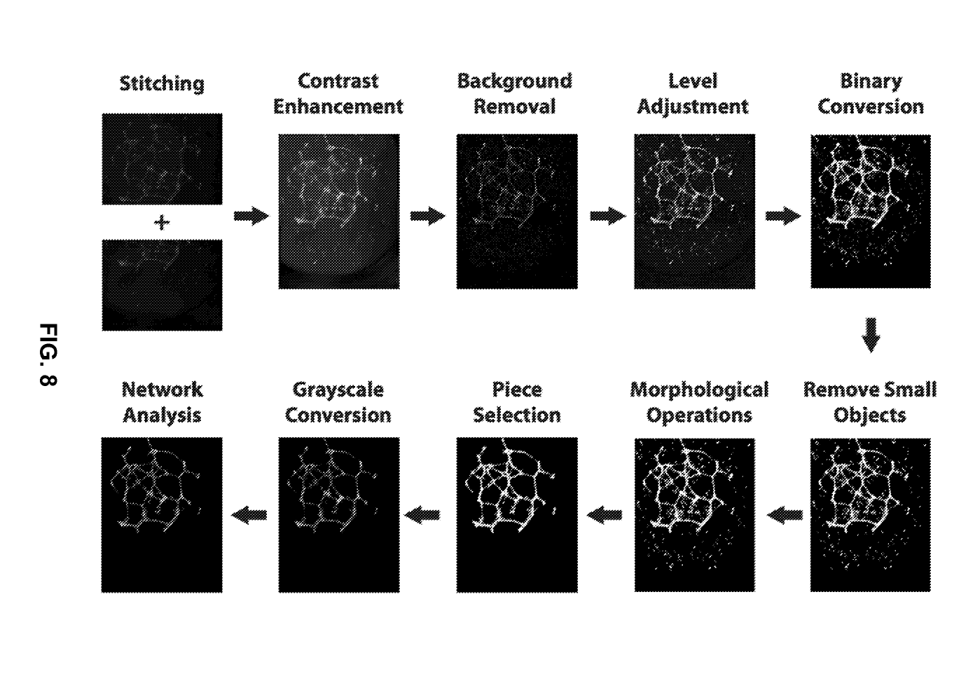

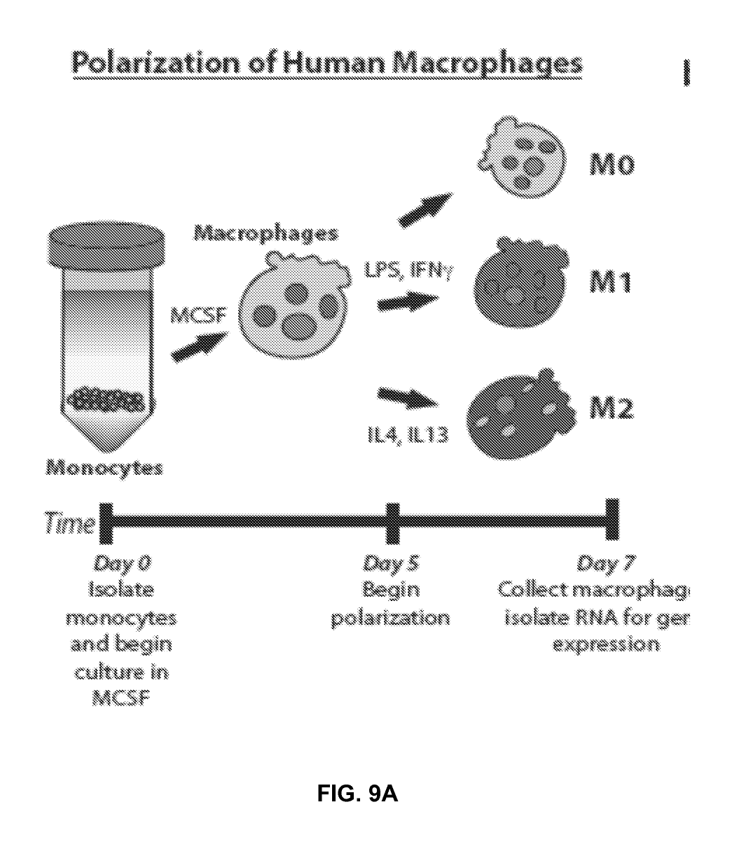

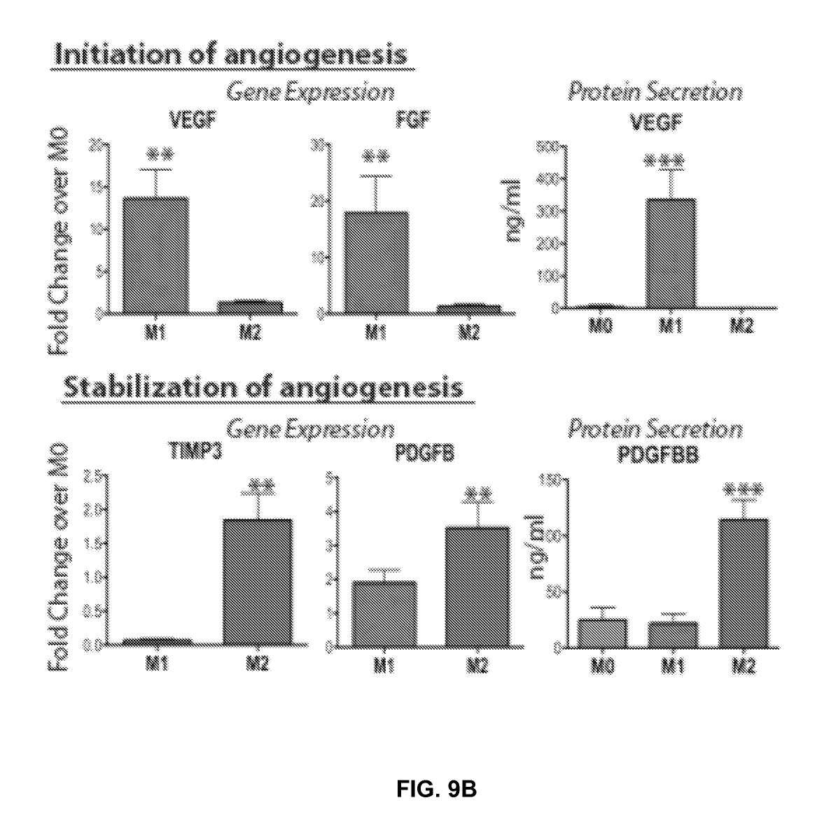

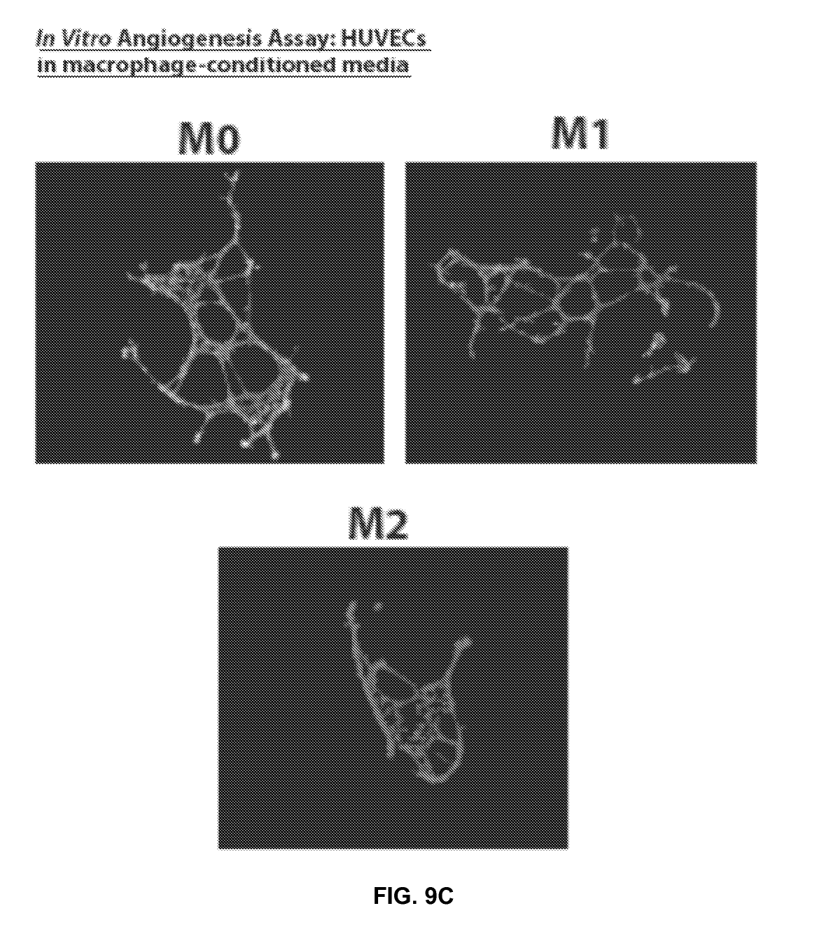

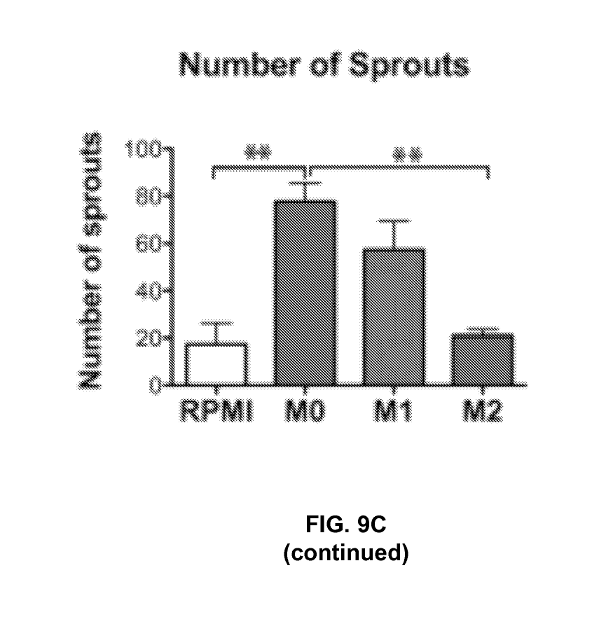

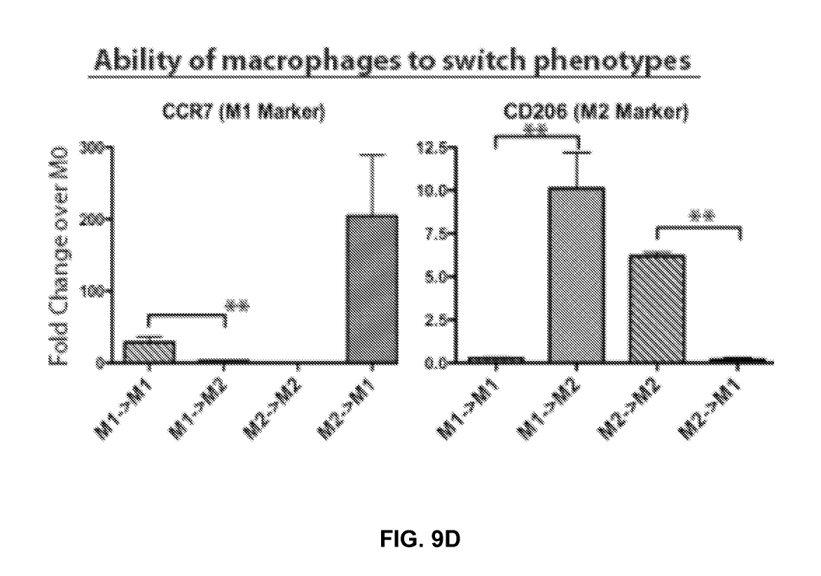

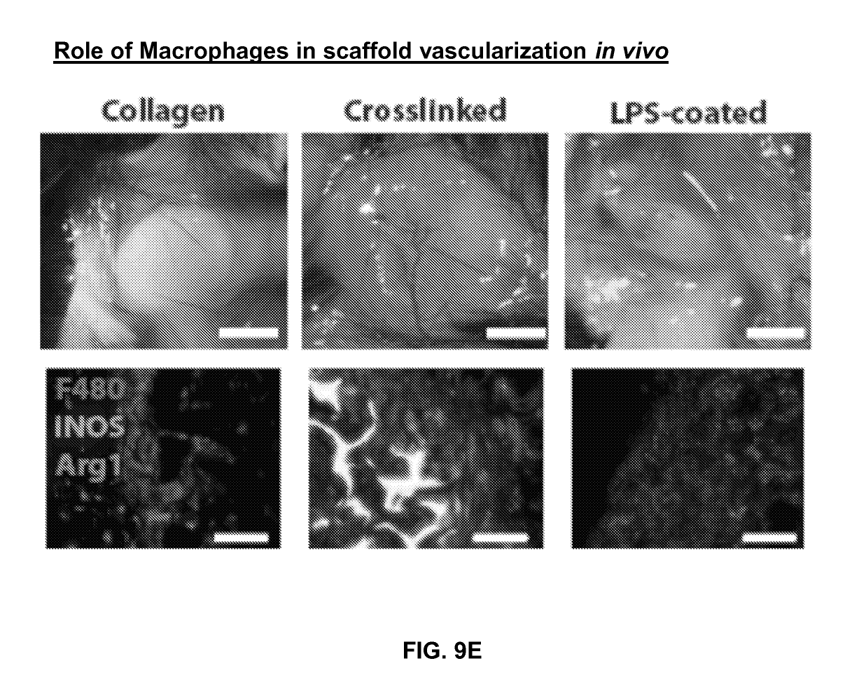

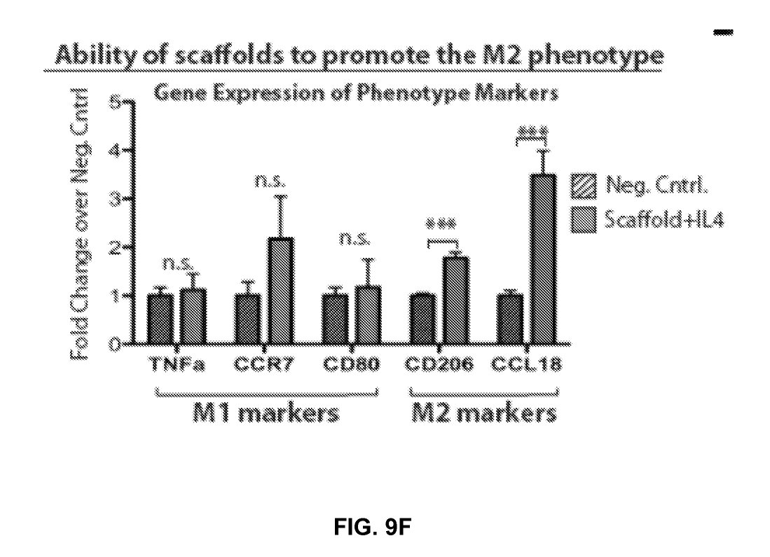

The following example demonstrates that scaffolds that promote the M1 phenotype of macrophages followed by the M2 phenotype can increase vascularization. Macrophages were differentiated from monocytes and polarized to different phenotypes (see e.g., FIG. 9A). M1 macrophages were shown to express and secrete growth factors important in early stages of angiogenesis, while M2 macrophages were shown to express and secrete growth factors important in later stages of angiogenesis (see e.g., FIG. 9B). Endothelial cells were shown to increase sprout formation in M0 and M1-conditioned media, but not M2-conditioned media (see e.g., FIG. 9C). Macrophages were shown to switch their phenotype from M1 to M2 or vice versa (see e.g., FIG. 9D). Both M1 and M2 macrophages were shown to be required for scaffold vascularization (see e.g., FIG. 9E). Scaffolds with conjugated IL4 where shown to cause M2 polarization of seeded macrophages (see e.g., FIG. 9F).

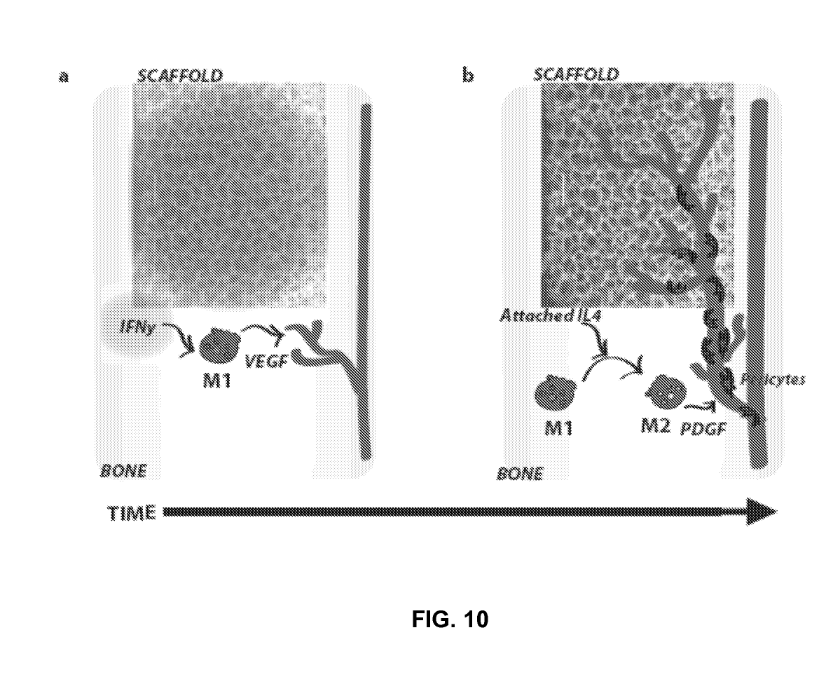

A scaffold was produced with attached IL4 and physically adsorbed IFNy, which would be cleared relatively quickly (˜1 day) from the scaffolds, thus promoting the M1 response followed by the M2 response. Adsorbed IFNy causes macrophages in the vicinity to polarize to the M1 phenotype (see e.g., FIG. 10A). They release angiogenic growth factors such as VEGF, recruit endothelial cells, and initiate the process of angiogenesis. When the adsorbed IFNy is cleared, the IL4 attached to the scaffold becomes exposed (see e.g., FIG. 10B). M1 macrophages convert to the M2 phenotype and secrete factors such as PDGF that recruit pericytes to stabilize the growing vasculature.

### Example 7

### Effects of IFNγ, IL4, or IL10 on Macrophage Phenotype

Macrophages were derived from monocytes isolated from human peripheral blood and polarized to the M1, M2a, or M2c phenotypes through the addition of lipopolysaccharide (LPS) and IFNγ (M1), interleukin-4 (IL4) and IL13 (M2a), or IL10 (M2c), respectively, using established methods (see Martinez et al. 2006 J. Immunol 177, 7303-7311). To probe the roles of different macrophage phenotypes in angiogenesis, human umbilical vein endothelial cells (HUVECs; at passage numbers less than 5) were cultured in medium conditioned by cultivation of polarized macrophages (M1, M2a) or unactivated macrophages (M0) for seven days on fibrin gel, then stained with fluorescin-phalloidin for actin filaments and visualized via confocal microscopy. Transwell migration of HUVECs towards macrophage-conditioned medium or cytokine controls was also assessed after 5 hrs (n=4). To explore the possibility of using biomaterials to control the macrophage phenotype, decellularized bone scaffolds were prepared (as in Grayson et al. 2009 PNAS 107(8), 3299-3304) and modified through the conjugation of IFNγ, IL4, or IL10, in order to elicit the M1, M2a, or M2c phenotype, respectively. This conjugation was achieved by joining biotinylated scaffolds and biotinylated proteins with streptavidin. Unactivated macrophages were seeded on these scaffolds (500,000 each) for 2 days and analyzed for gene expression by RT-PCR. Data are presented as mean±SEM and subjected to one-way analysis of variance or Student's T-test, as appropriate.

Results showed that conditioned media from M1 macrophages caused substantial alignment of HUVECs on fibrin gel, which was not seen in media from M0 or M2a macrophages. The sequential addition of conditioned media from M1 and M2 macrophages resulted in some network formation by HUVECs, which was not seen with either type of macrophage-conditioned media alone. Conditioned media from M0 and M1 macrophages inhibited transwell recruitment of HUVECs, while conditioned media from M2a macrophages significantly stimulated recruitment, when compared to cytokine controls (n=4, p<0.05). When unactivated macrophages were seeded on bone scaffolds modified with IFNγ, expression of the M1 phenotype marker CD80 was significantly increased (n=3, p<0.05)). Scaffolds modified with IL4 caused increased expression of the M2a marker CCL18, but this increase was not significant (n=3, p>0.05). Scaffolds modified with IL10 caused significant upregulation of the M2c marker CD163 (n=3, p<0.05).

These results support that both types of macrophages play distinct and important roles in angiogenesis, in a way that can be utilized to enhance bone vascularization. Scaffolds functionalized by incorporation of attached cytokines directed the phenotype of macrophages in vitro in a specific and controllable manner.

### Example 8

### Characterization of Macrophage Phenotype

The following example shows in vitro kinetics of macrophage phenotype switch using flow cytometry, gene expression, and cytokine secretion analysis.

Isolation and Culture of Primary Human Macrophages.

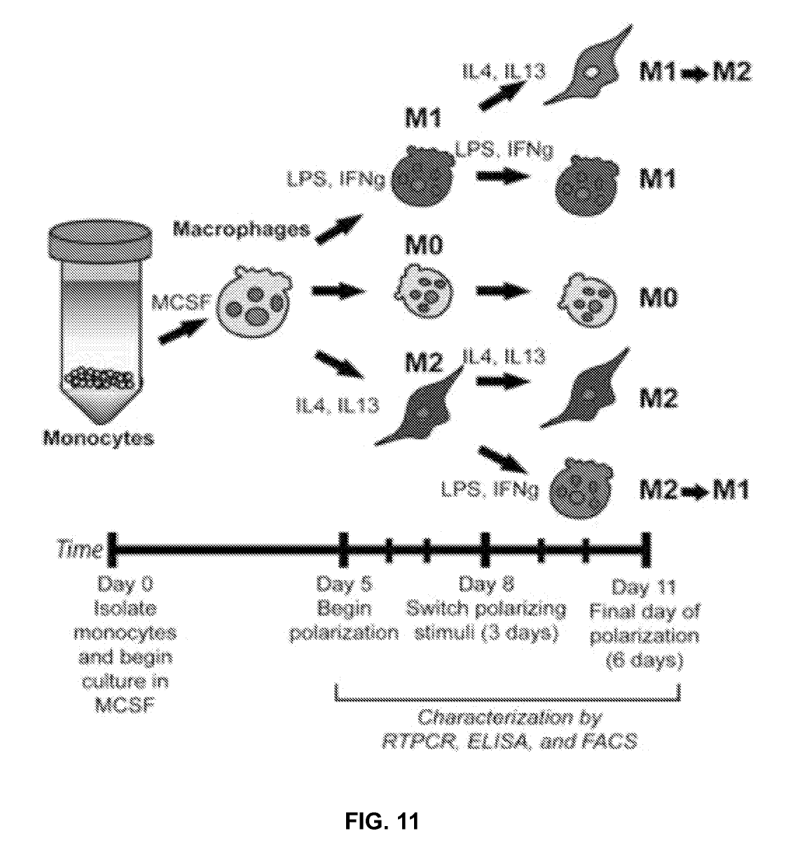

Monocytes were isolated from enriched leukocyte fractions of human peripheral blood purchased from the New York Blood Center using sequential Ficoll and Percoll density gradient centrifugations (as described in Spiller et al. 2014 Biomaterials 35(15), 4477-88). Monocytes were cultured at 37° C. and 5% CO2 in ultra low attachment flasks (Corning) for five days at a density of 0.4×106 cells/cm2 and 1.0×106 cells/ml of complete media (RPMI media supplemented with 10% heat-inactivated human serum, 1% penicillin-streptomycin, and 20 ng/ml macrophage colony stimulating factor (MCSF)). Macrophages were polarized over the next 1-6 days by culturing at 1.0×106 cells/ml in complete media with 100 ng/ml IFN-gamma (Peprotech, Rocky Hill, N.J.) and 100 ng/ml lipopolysaccharide (LPS, Sigma Aldrich) for M1 or 40 ng/ml IL4 and 20 ng/ml IL13 (Peprotech, Rocky Hill, N.J.) for M2, with a media change at day 3. At the media change, the media of another group of M1 macrophages was switched to M2-polarizing media and the media of a group of M2 macrophages was switched to M1-polarizing stimuli, in order to characterize the ability of macrophages to switch phenotypes. Unactivated macrophages were also cultured over the same time periods (M0), resulting in three groups through day 3 (M0, M1, M2) and five groups between days 4 and 6 (M0, M1, M2, M1→M2, M2→M1) (see e.g., FIG. 11).

Characterization of Macrophage Phenotype.

At days 1, 2, 3, 4 and 6, the macrophages were collected by gentle scraping and centrifugation. The number of viable cells was determined at each time point by trypan blue exclusion. Macrophages from each time point were characterized for expression of known M1 and M2 markers by flow cytometry and quantitative RT-PCR (as described in Spiller et al. 2014 Biomaterials 35(15), 4477-88). The supernatant was frozen at −80° C. until analysis by enzyme-linked immunosorbent assays (ELISA). Secreted M1 markers included tumor necrosis factor-alpha (TNF-alpha) and VEGF (Peprotech) and M2 markers included CCL18 (R&D Systems) and PDGF-BB (Peprotech).

Results for Kinetics of Macrophage Phenotype Switching.

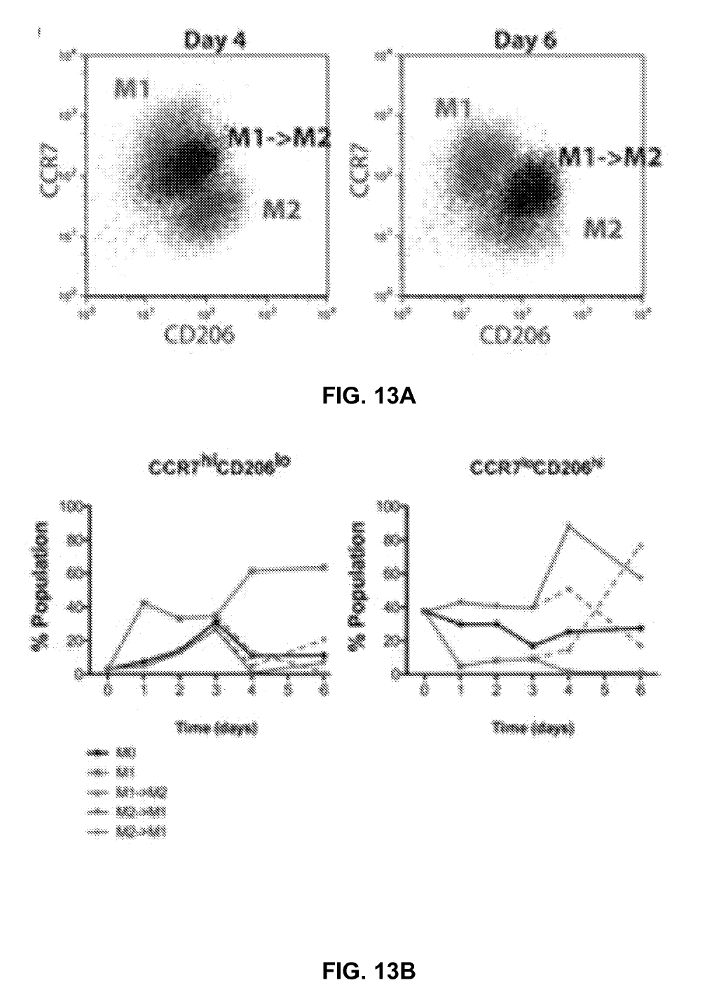

Over 6 days of culture in the presence of polarizing stimuli, M1 and M2 macrophages gradually increased surface marker expression of CCR7 and CD206, with M1 macrophages staining more strongly for CCR7 and M2 macrophages staining more strongly for CD206. When M1 macrophages were given M2-promoting stimuli at day 3, the entire population shifted to express less CCR7 and more CD206 (see e.g., FIG. 13A). Similarly, M2 macrophages that were given M1-promoting stimuli at day 3 showed reduced CD206 expression and increased CCR7 expression.

Maximum staining was observed at day 4, both in terms of the percentage of the population staining positively and the mean intensity per cell, which is a better indicator of macrophage phenotype than the percent of cells staining positively (see Spiller et al. 2014 Biomaterials 35(15), 4477-88). To more accurately describe the change in the numbers of cells representing the M1 and M2 populations, gating was performed based on the mean intensities of CD206 and CCR7 expression of the M0 population at the same time point, in order to determine the number of cells that could be described as CCR7hi CD206lo, which would indicate the M1 phenotype, and those that were CCR7lo CD206hi, which would be more indicative of the M2 phenotype (see e.g., FIG. 13B). Interestingly, the greatest changes in expression were seen at day 4, or one day after the media change at day 3, even for control phenotypes that were not switched, indicating that the macrophages were able to respond to increased stimulus. In addition, the change from M1→M2 appeared more dramatic than the change from M2→M1, in that the latter group did not show expression of CCR7 after 6 days at the same levels as M1 controls, even though M1→M2 cells showed levels of CD206 that were higher than M2 controls at day 6.

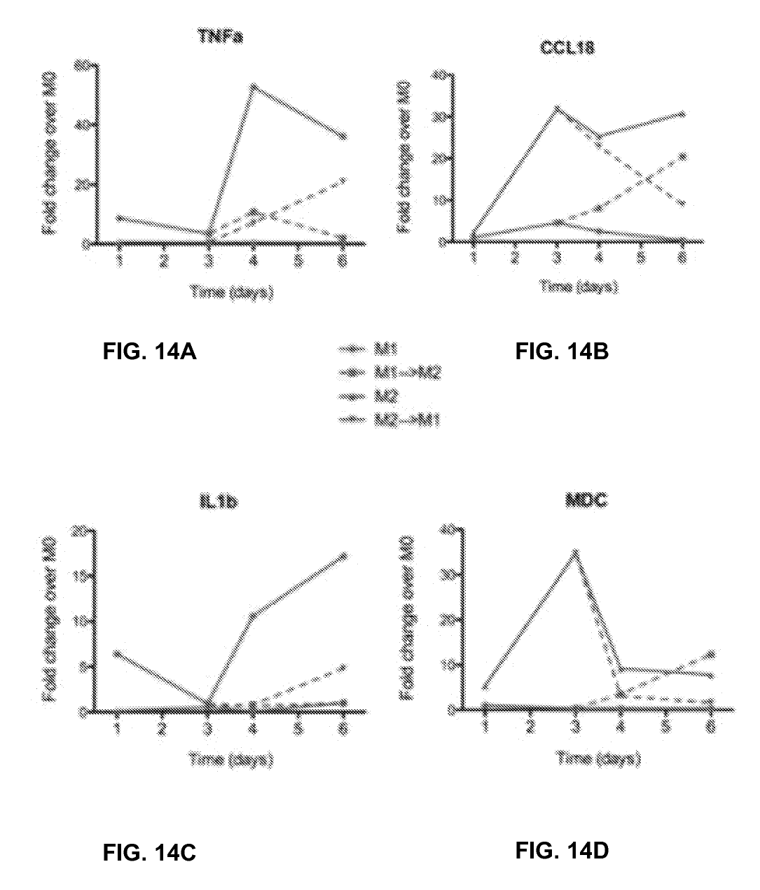

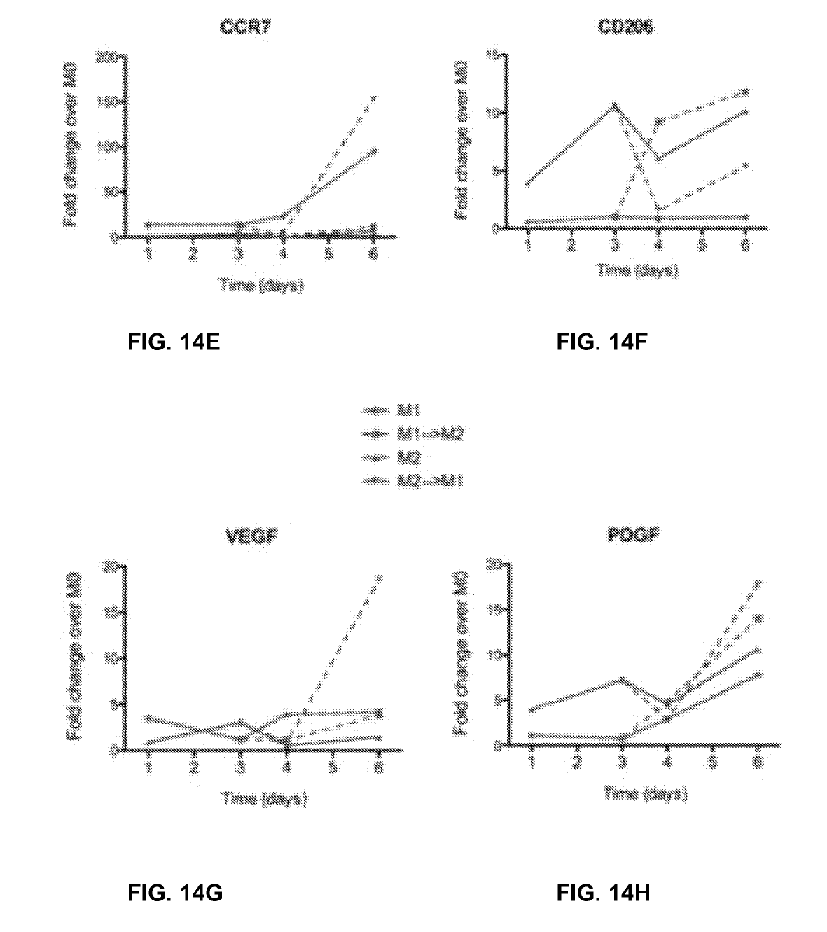

Gene expression of the M1 markers TNFa, IL1b, CCR7, and VEGF was highest for M1 macrophages and increased over time, with the highest expression at day 6 (see e.g., FIG. 14). In keeping with flow cytometry results, a dramatic increase was seen at day 4, after the media change. The addition of M2-promoting stimuli at day 3 effectively inhibited expression of these genes and caused upregulation of the M2 markers CCL18, MDC/CCL22, CD206/MRC1, PDGF, and TIMP3. M2 macrophages showed high levels of expression of the M2 markers, with maximum expression at day 3, until the media was changed to M1-polarizing stimuli, at which point they decreased expression of M2 markers and increased expression of M1 markers. Both M1 and M2 macrophages that were switched to the other phenotype expressed genes comparable to or higher than the control phenotypes.

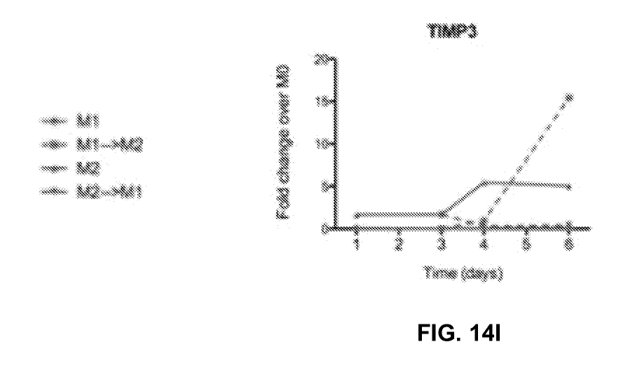

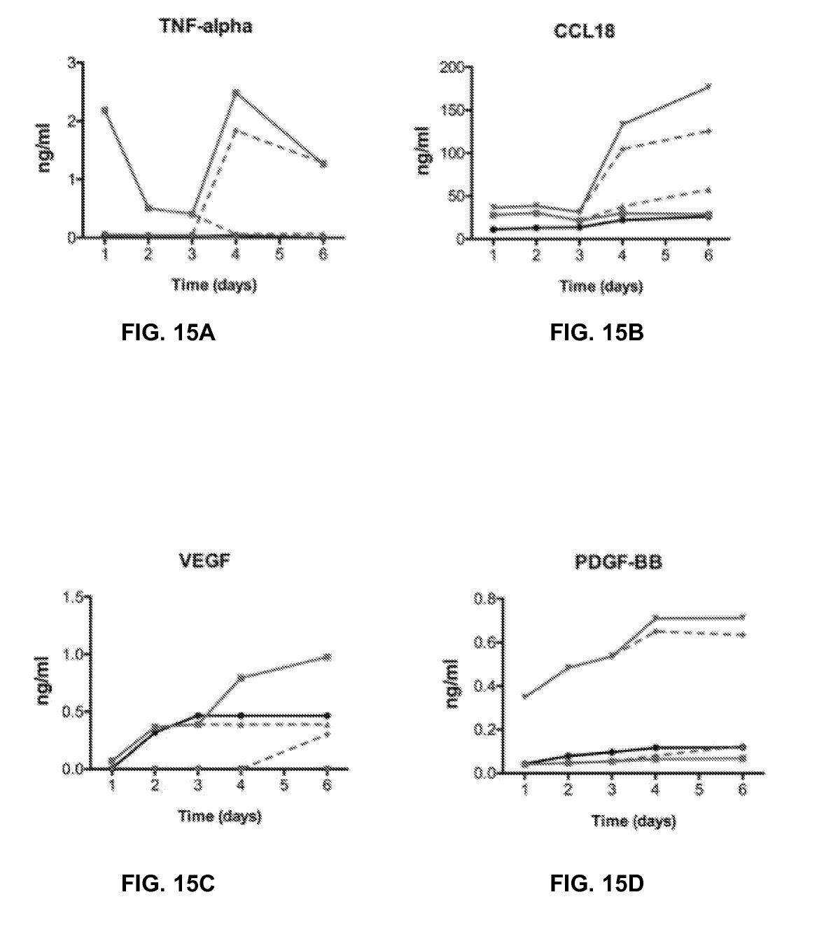

M1 macrophages secreted high levels of TNF-alpha and VEGF, with maximum secretion at days 4-6 (see e.g., FIG. 15). The addition of M2-polarizing stimuli caused drastic inhibition of secretion of these markers and increased in secretion of the M2 markers CCL18 and PDGF-BB, compared to control M1 macrophages that were stimulated for 6 days. Similarly, the addition of M1-polarizing stimuli to M2 macrophages caused decreased secretion of M2 markers CCL18 and PDGF-BB as well as increased secretion of the M1 markers TNF-alpha and VEGF.

Interestingly, M2 macrophages, including M1 macrophages that were switched to the M2 phenotype, proliferated over time in culture. When the amount of secreted proteins was normalized to the number of viable cells at each time point, the amounts of M2 markers secreted by M1 macrophages that were switched to M2 media were only slightly higher than the M1 control.

### Example 9

### Scaffolds

The following example shows scaffolds for bone regeneration based on modifications of decellularized bone for a short release of interferon-gamma (IFNg) to promote the M1 phenotype, followed by a more sustained release of interleukin-4 (IL4) to promote the M2 phenotype. To achieve this sequential release profile, IFNg was physically adsorbed onto the scaffolds, while IL4 was attached via biotinstreptavidin binding.

Methods were according to Example 8 unless indicated otherwise.

Preparation and Biotinylation of Scaffolds.

Decellularized bone scaffolds were prepared from trabecular bone of 4-8 week old cows by coring plugs from the subchondral regions and washing with water and detergents (as described in Grayson et al. 2010 Proc Natl Acad Sci USA. 107(8), 3299-304 and Spiller et al. 2014 Biomaterials 35(15), 4477-88). Scaffolds (4 mm in diameter×2.5 mm in height) were separated based on density that was calculated by measuring the height, diameter, and mass of cylindrical samples, in order to ensure uniformity between experiments. The average density of the scaffolds used in this study was 0.49±0.03 mg/mm³ (mean±standard deviation).

Scaffolds were sterilized by soaking in 70% ethanol for 24 hours, followed by washing in phosphate-buffered saline (PBS). Then, scaffolds were biotinylated using NHS (N-Hydroxysuccinimide) chemistry by immersion in 10 mM Biotin-sulfo-LC-LC-NHS (EZ Link™, Thermo Fisher Scientific, Rockford, Ill.) for one hour, followed by three washes with 2 ml PBS to remove unattached biotin. Scaffolds were briefly immersed again in 70% ethanol for 10 min, followed by three more washes, and finally immersed in PBS at 4° C. overnight prior to attachment of biotinylated proteins.

The extent of scaffold biotinylation was determined after mixing with avidin and HABA (4′-hydroxyazobenzene-2-carboxylic acid, Thermo Fisher Scientific, Rockford, Ill.). HABA binds strongly to avidin, but is displaced by biotin, which binds at a much higher affinity, causing a decrease in the absorbance of HABA, which can be read spectrophotometrically. A standard curve for biotin was prepared in a 96-well plate using non-biotinylated scaffolds together with 20 ul of biotin solutions ranging from 0 to 100 ug/ml. 180 ul of a solution of HABA and avidin (2.69 mg/ml HABA and 0.467 mg/ml avidin) was added to each well containing the standards or the biotinylated scaffolds. After 1 minute the scaffolds were removed and the absorbance was read at 500 nm. The difference in absorbance from blank controls was used to generate a standard curve and to calculate the amount of biotin on each scaffold.

In preliminary studies, an approximately 50-fold excess of biotin to protein content of the scaffolds (calculated using the assumption that the protein was 100% collagen) was found to result in the same level of biotinylation as up to 500-fold molar excess. Therefore a 50-fold molar excess of biotin was used for scaffold biotinylation.

Protein Biotinylation and Conjugation to Scaffolds.

IL4 was biotinylated by adding a 100-fold molar excess of the 10 mM Biotin-sulfo-LS-LS-NHS for one hour, followed by dialysis overnight to remove unattached biotin, and then sterile-filtered. Retention of bioactivity was 75%, determined using an IL4 ELISA (Peprotech).

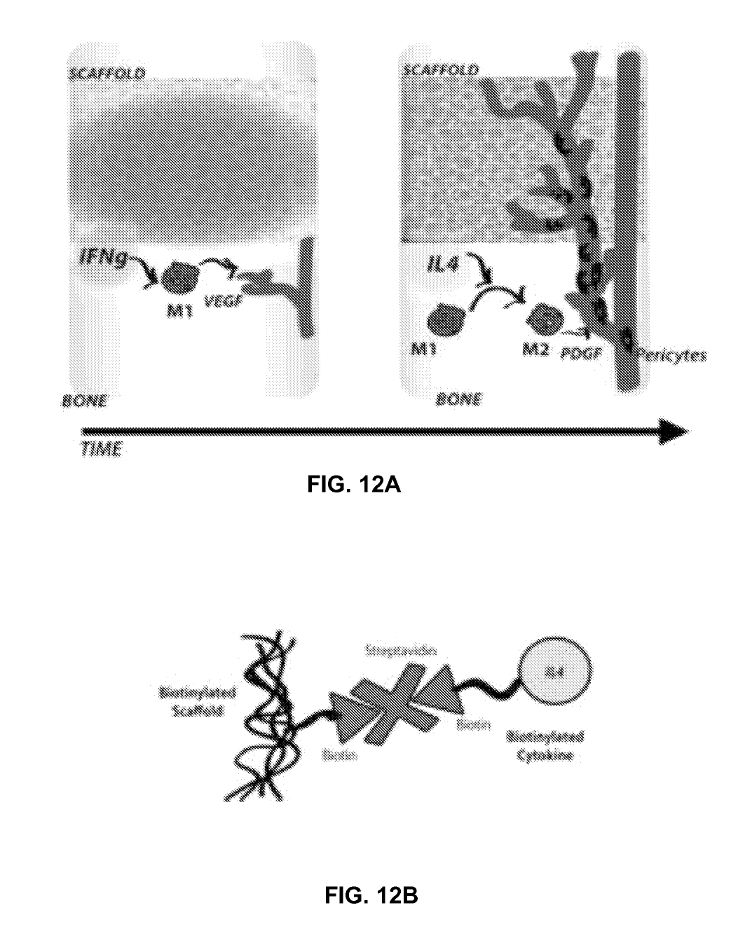

Four groups of scaffolds were prepared: scaffolds with attached IL4 (IL4), scaffolds with adsorbed IFN-gamma (IFNg), their combination (Combo), and a negative control (Neg. Cntrl), which was prepared in the same way as the other scaffolds but using PBS instead of IFN-gamma or IL4 solutions (See e.g., FIG. 12A).

For all groups, biotinylated scaffolds were soaked in 0.5 ml of 172 μg/ml streptavidin (Thermo Fisher Scientific) for 1 hour, followed by washing 3 times in PBS. To prepare the IL4 and Combo groups, scaffolds were soaked in 375 ng biotinylated IL4 in 0.5 ml of PBS for 1 hour, while Neg. Cntrl. and IFNy groups were soaked in PBS. Streptavidin has four binding sites for biotin with extremely high specificity and strength, creating a strong but not covalent linkage between IL4 and the scaffolds (see e.g., FIG. 12B). To determine that streptavidin bound specifically to biotin on the scaffolds, biotinylated scaffolds were incubated with fluorescent Streptavidin-DyLight-594 (Thermo Fisher Scientific) and compared to non-biotinylated scaffolds using confocal laser scanning microscopy.

Following streptavidin binding, scaffolds were washed 3 times with 2 ml PBS to remove unattached IL4. Then, scaffolds in the IFNy and Combo groups were incubated in IFN-gamma (325 ng/scaffold) for 1 hour to allow physical adsorption, while Neg. Cntrl. and IL4 scaffolds were soaked in PBS. Scaffolds were then transferred to 24-well ultra low attachment plates for release studies or for macrophage culture.

Characterization of Release Profiles.

To characterize the release of IFN-gamma and IL4 proteins from the scaffolds, scaffolds from each of the four groups were incubated in 1 ml complete media for 11 days at 37° C. and 5% CO2, with samples taken and media refreshed at 6 hrs, 1 day, 2 days, 3 days, 6 days and 11 days. The amount of IFN-gamma and IL4 in each sample was determined using ELISA (Peprotech). Values obtained for the negative control scaffolds were subtracted from the experimental groups at each time point.

Macrophage Seeding and Characterization

Macrophages were collected 5 days after differentiation from monocytes and seeded onto the scaffolds at 8.0×105 per scaffold in 20 μl of complete media (n=6). The cells were allowed to attach for 1 hour before the addition of 1 ml complete media. The cell-seeded constructs were cultured for 3 and 6 days, with a media change after 3 days. The media were frozen at −80° C. until analysis for M1 and M2 markers by ELISA, as described above. To extract RNA from the scaffolds, the scaffolds were immersed in 1 ml Trizol Reagent (Life Technologies) with 5-6 steel beads (0.5 mm diameter) and homogenized for 6 cycles of 10 seconds in a Mini Bead Beater-8 (Biospec Products, Bartlesville, Okla.). RNA was extracted into chloroform, which was then purified using an RNeasy Micro Kit (Qiagen) according to the manufacturer's instructions. DNase treatment, cDNA synthesis, and RT-PCR was performed (as described in Spiller et al. 2014 Biomaterials 35(15), 4477-88).

LPS Contamination.

Cell culture media were periodically tested for contamination with LPS using the LAL Chromogenic Endotoxin Quantification kit (Thermo Scientific Fisher) per the manufacturer's instructions. LPS contamination was always below 0.1 EU/ml.

Statistical Analysis.

Data are presented as mean±SEM. Data from all in vitro experiments are representative of at least three independent experiments. Statistical analysis was performed in GraphPad Prism 4.0 using one-way ANOVA and either Tukey's or Dunnett's post-hoc analysis, as indicated. A p-value of less than 0.05 was considered significant.

Results of release studies showed that despite the strong interactions between biotin and streptavidin, biotinylated IL4 was released over 6 days. These scaffolds promoted sequential M1 and M2 polarization of primary human macrophages as measured by gene expression of ten M1 and M2 markers and secretion of four cytokines, although the overlapping phases of IFNg and IL4 release tempered polarization to some extent.

Release of IFN-Gamma and IL4.

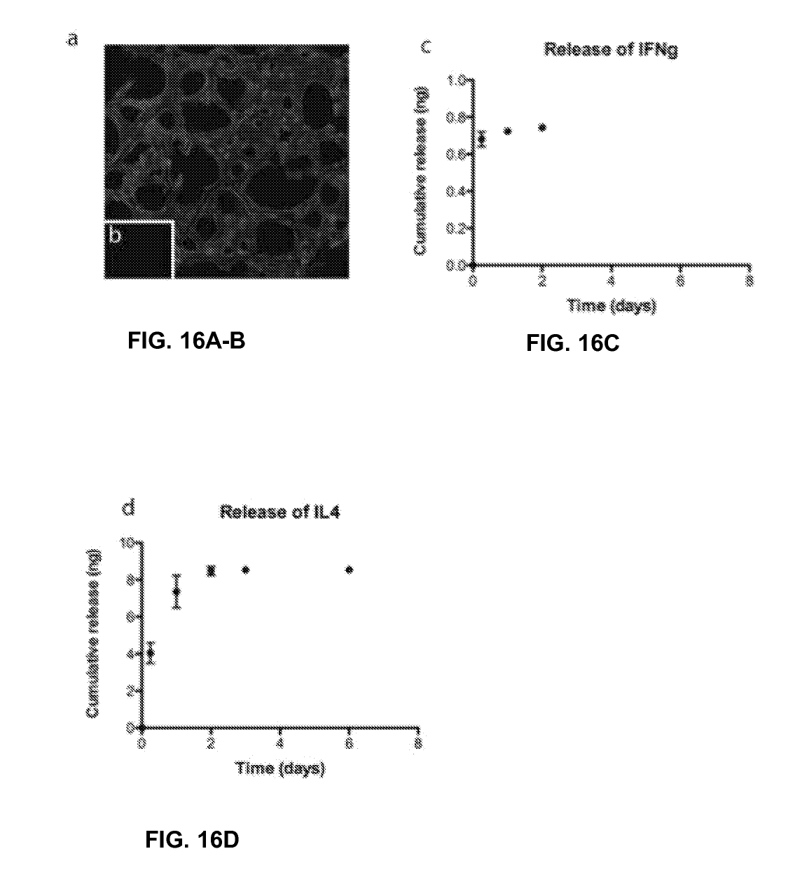

Decellularized bone scaffolds were biotinylated using NHS chemistry. Streptavidin was found to only bind to scaffolds that were biotinylated (FIG. 16A), with undetectable nonspecific binding to control scaffolds after washing (FIG. 16B).

Release studies showed that all of the adsorbed IFN-gamma was released in the first 48 hours, resulting in a concentration of less than 1 ng/ml in the media (FIG. 16C). Biotinylated IL4 was released over 6 days, with no detectable IL4 in the media after that point (FIG. 16D). Release profiles of IFN-gamma and of IL4 were not found to be different for Combo scaffolds, which had both IFNg and IL4, compared to the scaffolds with only IFN-gamma or IL4.

Response of Macrophages to Immunomodulatory Scaffolds

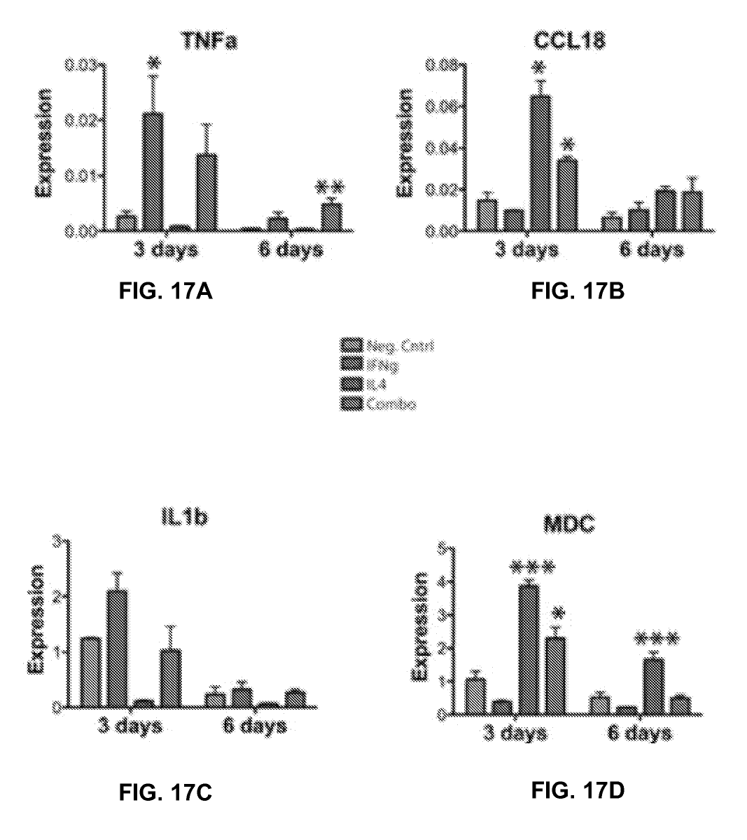

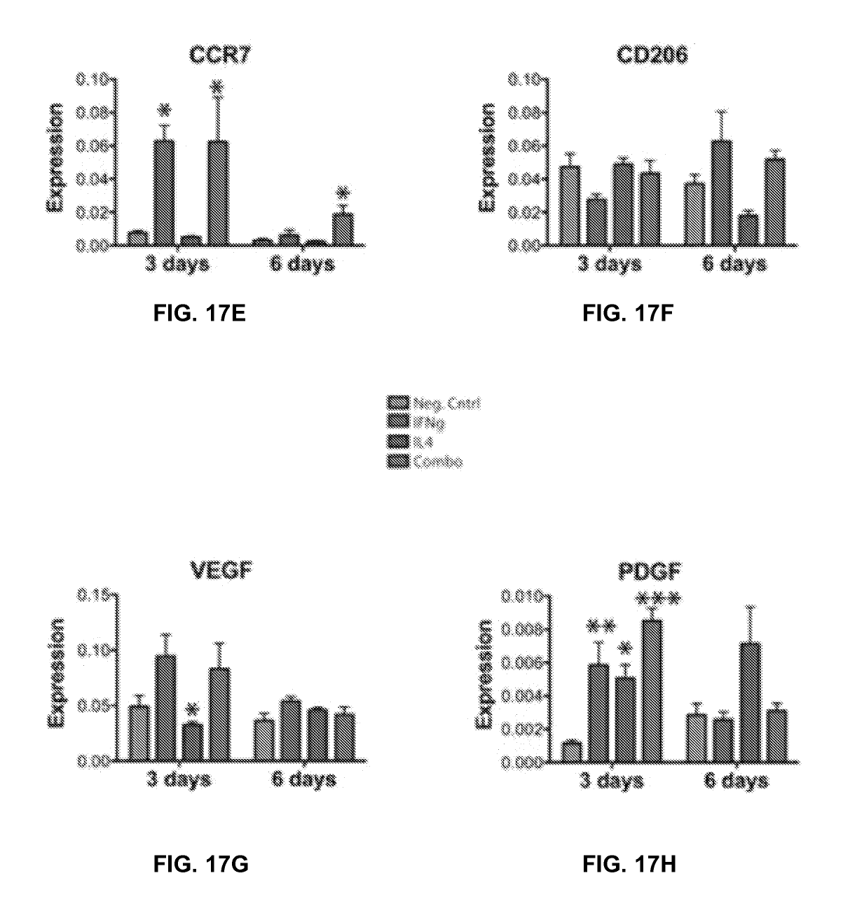

Gene expression data indicated that physical adsorption of IFN-gamma to scaffolds with and without attached IL4 caused increased expression of M1 markers after 3 days of culture (see e.g., FIG. 17). This early M1 polarization was achieved despite low levels of protein released in the first three days (less than 1 ng, compared to the dose of 100 ng that is typically used to polarize macrophages to the M1 phenotype (see e.g., FIG. 16C). Expression of M1 markers decreased to background levels by day 6, although expression of TNFa and CCR7 did remain significantly higher for Combo scaffolds compared to the negative control. At both 3 and 6 days, expression of M2 markers was significantly higher for macrophages seeded on scaffolds with attached IL4 compared to the negative control. Macrophages seeded on scaffolds in the Combo group also significantly increased gene expression of M2 markers at day 3, but these increases were not significant at day 6.

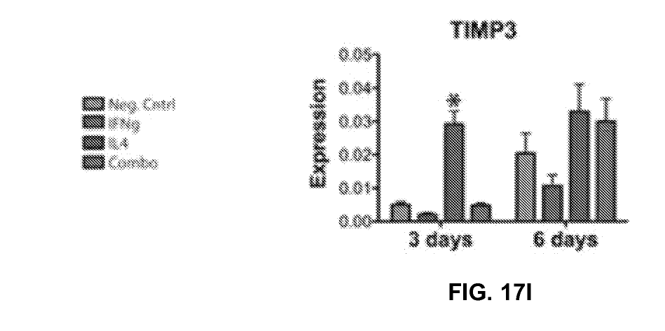

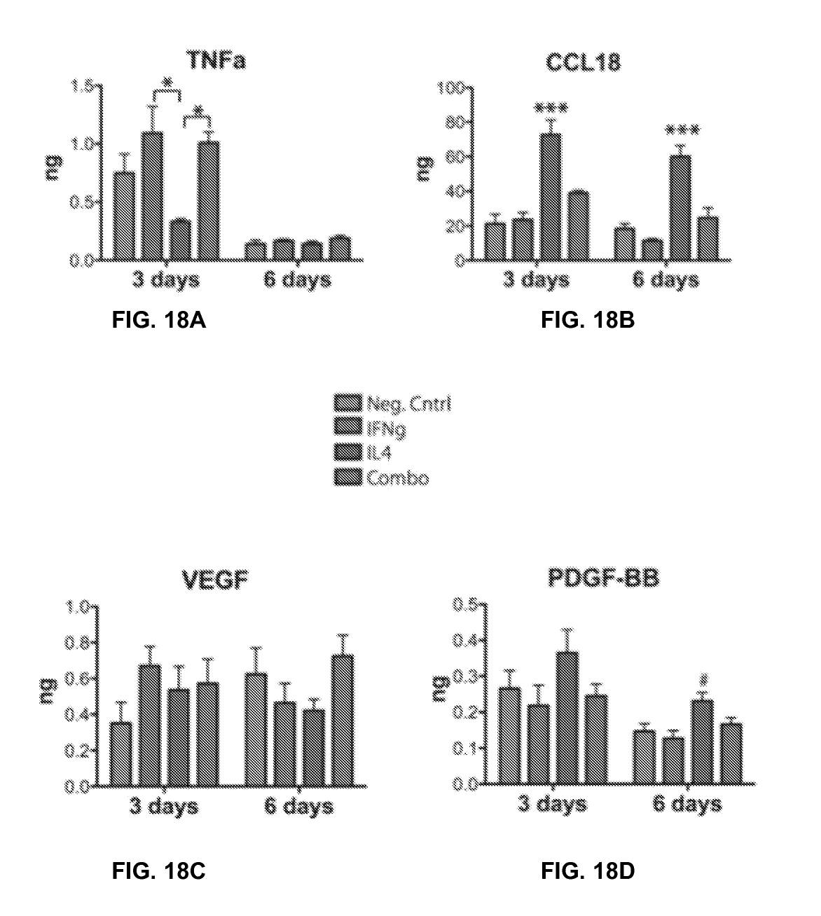

The amounts of secreted proteins associated with the M1 and M2 phenotypes were measured using ELISA to confirm gene expression results. Adsorption of IFN-gamma caused increases in the secretion of the M1 marker TNF-alpha at 3 days compared to the IL4 group (one-way ANOVA with Tukey's post-hoc analysis, p<0.05); comparable differences were observed in negative control (see e.g., FIG. 18). No differences were seen in M1 marker secretion at 6 days. Attachment of IL4, without adsorbed IFNg, caused significant increases in secretion of the M2 marker CCL18, which was sustained at 6 days (one-way ANOVA with Tukey's post-hoc analysis, p<0.001). Attachment of IL4 also increased secretion of PDGF-BB at 6 days compared to the negative control (one-way ANOVA with Dunnett's post-hoc analysis, p<0.05). Interestingly, macrophages seeded on the Combo scaffolds did not show significantly different levels of secretion of any marker compared to the control, despite their ability to promote changes in both M1 and M2 gene expression.

### Example 10

### Murine Subcutaneous Implantation Model

The following examples shows scaffolds with physically absorbed IFNg and biotinstreptavidin bound IL4 subcutaneously implanted into a murine model.

Methods were according to Examples 8-9 unless indicated otherwise.

Subcutaneous Implantation Model.

Scaffolds were prepared as described above except using murine cytokines (Peprotech). One scaffold from each of the four groups was implanted subcutaneously in female 8-week-old C57BL/6 mice for two weeks (n=3 mice). Mice received a subcutaneous injection of buprenorphine (0.1 mg/ml) for pain, anesthetized using isofluorane (1-5%), shaved, cleaned with ethanol and iodine, and then draped for surgery. A small incision was made in the central dorsal surface using a scalpel. Blunt forceps were used to create a pocket in the subcutaneous space for the scaffolds. After implantation, wounds were closed with one wound clip. Mice were housed together and monitored for 14 days. No signs of pain or discomfort were observed following surgery or throughout the study.

Following 2 weeks of in vivo cultivation, mice were euthanized by CO2 asphyxiation. Scaffolds were explanted, fixed overnight in 4% paraformaldehyde, decalcified in formic acid (Immunocal, Decal Chemical Corporation, Tallman, N.Y.), dehydrated through an ethanol series and embedded in paraffin. Samples were sectioned to 5 μm and stained for general structure using hematoxylin and eosin (H&E). Endothelial cells were visualized via immunohistochemical staining for CD31. Sections were subjected to antigen retrieval by immersion in 95° C. citrate buffer for 20 min, then blocked for 1 hr in 5% bovine serum albumin, then incubated overnight with goat-anti-mouse CD31 (dilution 1:30, Santa Cruz Biotechnology, catalog no. sc-1506) and visualized using a donkey-anti-goat secondary antibody conjugated to FITC (Santa Cruz Biotechnology, catalog no. sc-2024), counterstained with DAPI (Vector Labs DAPI mounting medium). Fluorescent images of CD31 staining were acquired on an Evos FI Digital inverted fluorescence microscope. The intensity of CD31 staining of the cells within the samples was quantified in at least six images per section (10× magnification) and two sections per sample using ImageJ. The mean fluorescence intensity of the delete primary negative control was subtracted from that of the samples.

Samples were also analyzed for the presence of IL4 using rabbit-anti-mouse IL4 (1:10 Thermo Scientific Pierce, catalog no. PA 525165) and goat-anti-rabbit secondary antibody conjugated to DyLight488 (Thermo Scientific Pierce).

Results from the murine subcutaneous implantation model showed increased vascularization in scaffolds releasing IFNg compared to controls.

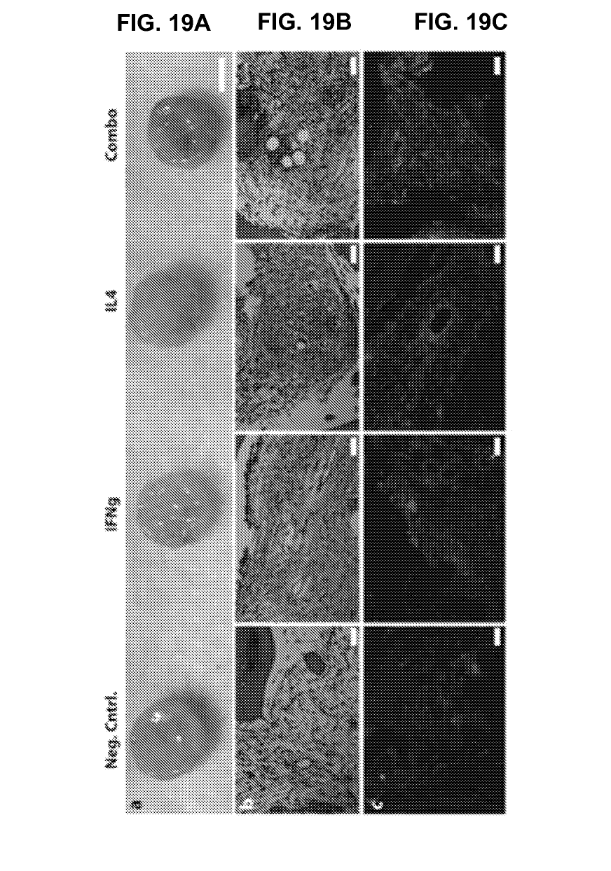

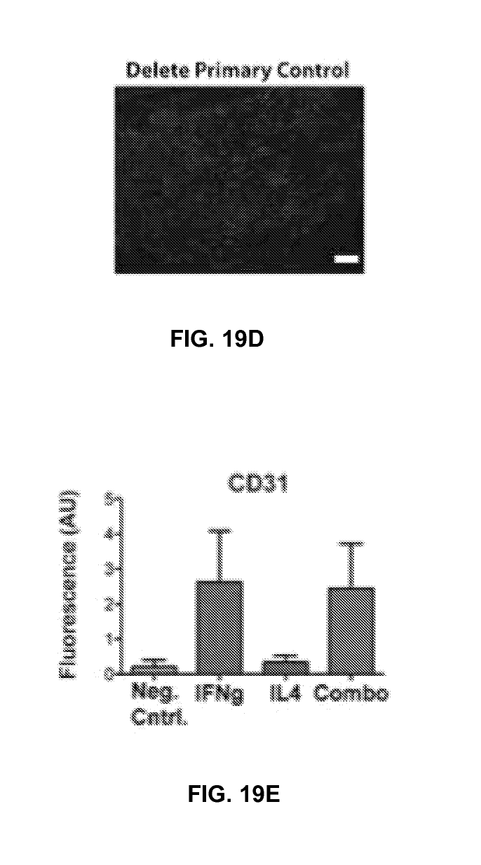

After 2 weeks of in vivo implantation (see e.g., FIG. 19A), all scaffolds were fully infiltrated by cells (see e.g., FIG. 19B). Large blood vessel-like structures were apparent in the IFNg, IL4, and Combo groups, but not in the negative control scaffolds. The endothelial cell marker CD31 was most abundant in IFNg and Combo samples (see e.g., FIG. 19C). The mean fluorescence intensities of CD31-stained cells in the negative control and IL4 scaffolds were not significantly different from the delete primary control (Student's t-test, p>0.05), indicating a lack of endothelial cell infiltration. In contrast, CD31 staining of cells in the IFNg and Combo scaffolds was higher than in the negative control (p<0.05). After subtracting background levels of intensity of the delete primary control from the experimental samples, intensity was slightly higher for the IFNg and Combo scaffolds compared to the control and IL4 scaffolds, but there were no statistically significant differences between any of the groups (n=3, one way ANOVA, p>0.05) (see e.g., FIG. 19E).

Murine IL4 was detected in all of the samples, without differences in staining between the groups, indicating that no scaffold-derived IL4 remained after 2 weeks in vivo.

1. A biocompatible scaffold comprising:
a matrix material;
a first composition that promotes an M1 macrophage phenotype; and
a second composition that promotes an M2 macrophage phenotype;
wherein the scaffold promotes an increased level vascularization when in fluid communication with cells in vitro or in vivo compared to a scaffold not comprising the first composition and the second composition.

2. The scaffold of claim 1, wherein:
the first composition comprises interferon-gamma (IFNy), lipopolysaccharide (LPS), or Tumor necrosis factor alpha (TNFα); or
the second composition comprises interleukin-4 (IL4), interleukin-13 (IL13), or interleukin-10 (IL10); and
optionally, the scaffold further comprises a third composition, the third composition comprising Interleukin-10 (IL10):
wherein,
if the third composition is present, the second composition comprises interleukin-4 (IL4) or interleukin-13 (IL13); and
the scaffold promotes an increased level vascularization when in fluid communication with cells in vitro or in vivo compared to a scaffold not comprising the first composition the second composition, and the third composition (when present).

3. The scaffold of claim 2, wherein
the first composition comprises interferon-gamma (IFNy), lipopolysaccharide (LPS), or Tumor necrosis factor alpha (TNFα) and promotes an M1 macrophage phenotype;
the second composition comprises interleukin-4 (IL4) or interleukin-13 (IL13) and promotes an M2A macrophage phenotype; and
the third composition comprises Interleukin-10 (IL10) and promotes an M2C macrophage phenotype.

4. The scaffold of claim 2, wherein,
the first composition is released prior to the second composition or the third composition (when present);
promotion of the M1 macrophage phenotype is temporally separated from promotion of the M2 macrophage phenotype; or
an effect of the M1 macrophage phenotype occurs prior to an effect of the M2 macrophage phenotype.

5. The scaffold of claim 2, wherein the first composition, the second composition, or the third composition (when present) is bound to the matrix.

6. The scaffold of claim 2, wherein the first composition, the second composition, or the third composition (when present) is releasably bound to the matrix.

7. The scaffold of claim 2, wherein the first composition, the second composition, or the third composition (when present) is adsorbed into or onto the matrix but not covalently bound.

8. The scaffold of claim 2, wherein
at least the first composition is adsorbed into or onto the matrix but not covalently bound;
the second composition or the third composition (when present) is releasably bound to the matrix; and
the first composition is released prior to the second composition or the third composition (when present).

9. The scaffold of claim 2, wherein:
IFNy is present in or on the scaffold at concentration of about 100 ng/ml;
LPS is present in or on the scaffold at concentration of about 100 ng/ml;
TNFα is present in or on the scaffold at concentration of about 100 ng/ml;
IL4 is present in or on the scaffold at concentration of about 40 ng/ml;
IL13 is present in or on the scaffold at concentration of about 20 ng/ml; or
IL10 is present in or on the scaffold at concentration of about 40 ng/ml.

10. The scaffold of claim 2, wherein the first composition, the second composition, or the third composition (when present) is formulated as a controlled release composition.

11. The scaffold of claim 2, wherein the first composition, the second composition, or the third composition (when present) is encapsulated in a polymeric microsphere or a liposome.

12. The scaffold of claim 1, further comprising cells.

13. The scaffold of claim 12, further comprising progenitor cells.

14. The scaffold of claim 12, further comprising cells selected from the group consisting of mesenchymal stem cells (MSC), MSC-derived cells, osteoblasts, chondrocytes, myocytes, adipocytes, neurons, glial cells, fibroblasts, cardiomyocytes, liver cells, kidney cells, bladder cells, beta-pancreatic islet cell, odontoblasts, dental pulp cells, periodontal cells, tenocytes, lung cells, cardiac cells, hematopoietic stem cells (HSC), HSC endothelial cells, blood vascular endothelial cells, lymph vascular endothelial cells, cultured endothelial cells, primary culture endothelial cells, bone marrow stem cells, cord blood cells, human umbilical vein endothelial cell (HUVEC), lymphatic endothelial cell, endothelial pregenitor cell, stem cells that differentiate into an endothelial cells, smooth muscle cells, interstitial fibroblasts, and myofibroblasts, or a combination thereof.

15. The scaffold of claim 12, wherein the cells are present in the matrix at a density of at least about 0.0001 million cells (M) ml⁻¹ up to about 1000 M ml⁻¹.

16. The scaffold of claim 1, wherein the matrix comprises a material selected from the group consisting of fibrin, fibrinogen, a collagen, a polyorthoester, a polyvinyl alcohol, a polyamide, a polycarbonate, a polyvinyl pyrrolidone, a marine adhesive protein, a cyanoacrylate, and a polymeric hydrogel, or a combination thereof.

17. A method of treating a tissue or organ defect comprising:
placing the scaffold of claim 2 into fluid communication with cells of a subject in need thereof;
wherein the scaffold produces an increased level vascularization compared to a scaffold not comprising the first composition, the second composition, or the third composition (when present).

18. The method of claim 17, further comprising incubating the scaffold in vitro, wherein the scaffold comprises cells.

19. The method of claim 17, wherein
(a) the first composition comprises interferon-gamma (IFNy), lipopolysaccharide (LPS), or Tumor necrosis factor alpha (TNFα) and promotes an M1 macrophage phenotype;
the second composition comprises interleukin-4 (IL4) or interleukin-13 (IL13) and promotes an M2A macrophage phenotype; and
the third composition comprises Interleukin-10 (IL10) and promotes an M2C macrophage phenotype; and
(b) the first composition is released prior to the second composition or the third composition;
promotion of the M1 macrophage phenotype is temporally separated from promotion of the M2A macrophage phenotype or the M2C macrophage phenotype; or
an effect of the M1 macrophage phenotype occurs prior to an effect of the M2A macrophage phenotype or the M2C macrophage phenotype.

20. The method of claim 17, wherein the subject is a horse, cow, dog, cat, sheep, pig, mouse, rat, monkey, hamster, guinea pig, and chicken, or human.
